(19) United States
(12) Patent Application Publication
(10) Pub. No.: US 2010/0256124 A1
(43) Pub. Date: Oct. 7, 2010

(54) Substituted 1-Methyl-1H-Quinolin-2-Ones And 1-Methyl-1H-1,5-Naphthyridin-2-Ones As Antibacterials

(76) Inventors: David Thomas Davies, Essex (GB); Graham Elgin Jones, Essex (GB); Neil David Pearson, Essex (GB)

Correspondence Address: GlaxoSmithKline;GLOBAL PATENTS -US, UW2220, P. O. BOX 1539, KING OF PRUSSIA, PA, 19406-0939, US

(21) Appl. No.: 12/303,997

(22) PCT Filed: Jun. 8, 2007
(86) PCT No.: PCT/EP2007/055643

§ 371 Date: Dec. 9, 2008

(30) Foreign Application Priority Data
Jun. 9, 2006 (GB) 0611470.6
Mar. 30, 2007 (GB) 0706290.4

(51) Int. Cl. A61K 31/542; C07D 498/04; A61K 31/5365; C07D 471/04; A61K 31/4375; C07D 513/04; A61P 31/04

(52) U.S. Cl. 514/224.2; 544/105; 514/230.5; 546/114; 514/301; 544/48

(57) ABSTRACT

Bicyclic nitrogen containing compounds and their use as antibacterials.

This invention relates to novel compounds, compositions containing them and their use as antibacterials.

WO02/08224, WO02/50061, WO02/56882, WO02/96907, WO2003087098, WO2003010138, WO2003064421, WO2003064431, WO2004002992, WO2004002490, WO2004014361, WO2004041210,WO2004096982, WO2002050036, WO2004058144, WO2004087145, WO06002047, WO06014580, WO06010040, WO06017326, WO06012396, WO06017468, WO06020561, WO01/25227, WO02/40474, WO02/07572, WO2004035569, WO2004089947, WO04024712, WO04024713, WO04087647, WO2005016916, WO2005097781, WO06010831, WO04035569, WO04089947, WO06021448, WO06032466 and WO06038172 disclose quinoline, naphthyridine, morpholine, cyclohexane, piperidine and piperazine derivatives having antibacterial activity.

This invention provides a compound of formula (I) or a pharmaceutically acceptable salt, solvate or N-oxide thereof:

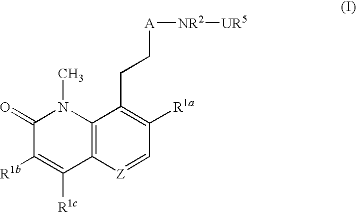

wherein:
Z is C or N;
R^{1a}, R^{1b} and R^{1c} are independently selected from hydrogen; halogen; cyano; (C₁₋₆)alkyl; (C₁₋₆)alkylthio; trifluoromethyl; trifluoromethoxy; carboxy; hydroxy optionally substituted with (C₁₋₆)alkyl or (C₁₋₆)alkoxy-substituted(C₁₋₆)alkyl; (C₁₋₆)alkoxy-substituted(C₁₋₆)alkyl; hydroxy (C₁₋₆)alkyl; an amino group optionally N-substituted by one or two (C₁₋₆)alkyl, formyl, (C₁₋₆)alkylcarbonyl or (C₁₋₆)alkylsulphonyl groups; or aminocarbonyl wherein the amino group is optionally substituted by (C₁₋₄)alkyl; provided that when Z is N, R^{1a} is not fluoro;
R² is hydrogen, or (C₁₋₄)alkyl, or together with R⁶ forms Y as defined below;
A is a group (i):

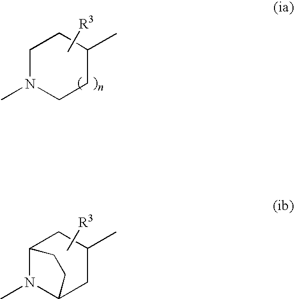

in which: R³ is as defined for R^{1a} or R^{1b} or is oxo and n is 1 or 2:
or A is a group (ii)

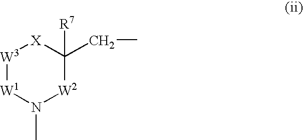

W¹, W² and W³ are CR⁴R⁸

or W² and W³ are CR⁴R⁸ and W¹ represents a bond between W³ and N.

X is O, CR⁴R⁸, or NR⁶;

one R⁴ is as defined for R^{1a}, R^{1b} and R^{1c} and the remainder and R⁸ are hydrogen or one R⁴ and R⁸ are together oxo and the remainder are hydrogen;

R⁶ is hydrogen or (C₁₋₆)alkyl; or together with R² forms Y;

R⁷ is hydrogen; halogen; hydroxy optionally substituted with (C₁₋₆)alkyl; or (C₁₋₆)alkyl;

Y is CR⁴R⁸CH₂; CH₂CR⁴R⁸; (C═O); CR⁴R⁸; CR⁴R⁸(C═O); or (C═O)CR⁴R⁸;

or when X is CR⁴R⁸, R⁸ and R⁷ together represent a bond;
U is selected from CO, and CH₂ and
R⁵ is an optionally substituted bicyclic carbocyclic or heterocyclic ring system (B):

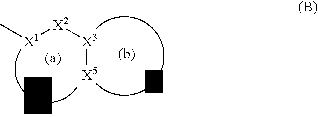

containing up to four heteroatoms in each ring in which

at least one of rings (a)and (b) is aromatic;

X¹ is C or N when part of an aromatic ring, or CR¹⁴ when part of a non-aromatic ring;

X² is N, NR¹³, O, S(O)ₓ, CO or CR¹⁴ when part of an aromatic or non-aromatic ring or may in addition be CR¹⁴R¹⁵ when part of a non aromatic ring;

X³ and X⁵ are independently N or C;

Y¹ is a 0 to 4 atom linker group each atom of which is independently selected from N, NR¹³, O, S(O)ₓ, CO and CR¹⁴ when part of an aromatic or non-aromatic ring or may additionally be CR¹⁴R¹⁵ when part of a non aromatic ring;

Y² is a 2 to 6 atom linker group, each atom of Y² being independently selected from N, NR¹³, O, S(O)ₓ, CO, CR¹⁴ when part of an aromatic or non-aromatic ring or may additionally be CR¹⁴R¹⁵ when part of a non aromatic ring;

each of R¹⁴ and R¹⁵ is independently selected from: H; (C₁₋₄)alkylthio; halo; carboxy(C₁₋₄)alkyl; (C₁₋₄)alkyl; (C₁₋₄)alkoxycarbonyl; (C₁₋₄)alkylcarbonyl; (C₁₋₄)alkoxy (C₁₋₄)alkyl; hydroxy; hydroxy(C₁₋₄)alkyl; (C₁₋₄)alkoxy; nitro; cyano; carboxy; amino or aminocarbonyl optionally mono- or di-substituted by (C₁₋₄)alkyl; or

R¹⁴ and R¹⁵ may together represent oxo;

each R¹³ is independently H; trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; (C₁₋₄) alkoxycarbonyl; (C₁₋₄)alkylcarbonyl; (C₁₋₆)alkylsulphonyl; aminocarbonyl wherein the amino group is optionally mono or disubstituted by (C₁₋₄)alkyl;

each x is independently 0, 1 or 2.

This invention also provides a method of treatment of bacterial infections in mammals, particularly in man, which method comprises the administration to a mammal in need of such treatment an effective amount of a compound of formula (I), or a pharmaceutically acceptable salt, solvate or N-oxide thereof.

The invention also provides the use of a compound of formula (I), or a pharmaceutically acceptable salt, solvate or N-oxide thereof, in the manufacture of a medicament for use in the treatment of bacterial infections in mammals.

The invention also provides a pharmaceutical composition comprising a compound of formula (I), or a pharmaceutically acceptable salt, solvate or N-oxide thereof, and a pharmaceutically acceptable carrier.

In a particular aspect each R^{1a}, R^{1b} and R^{1c} is independently hydrogen, (C₁₋₄)alkoxy, (C₁₋₄)alkylthio, (C₁₋₄)alkyl, cyano, carboxy, hydroxymethyl or halogen; more particularly hydrogen, methoxy, methyl, cyano, or halogen.

In some embodiments only one group R^{1a}, R^{1b} or R^{1c} is other than hydrogen. In a particular embodiment R^{1a} is methoxy, cyano or halo such as chloro or fluoro and R^{1b} and R^{1c} are hydrogen.

In a particular aspect R² is hydrogen.

Particular examples of R³ include hydrogen; optionally substituted hydroxy; optionally substituted amino; halogen; (C₁₋₄) alkyl; 1-hydroxy-(C₁₋₄) alkyl; optionally substituted aminocarbonyl. More particular R³ groups are hydrogen; CONH₂; 1-hydroxyalkyl e.g. CH₂OH; optionally substituted hydroxy e.g. methoxy; optionally substituted amino; and halogen, in particular fluoro. Most particularly R³ is hydrogen, hydroxy or fluoro.

In a particular aspect, when A is (ia), n is 1. In a further aspect, R³ is in the 3- or 4-position, more particularly in the 3-position. In a more particular aspect, A is (ia), n is 1 and R³ is in the 3-position, and more particularly is cis to the NR² group.

In a more particular aspect, A is (ia), n is 1 and R3 is H or hydroxy in the 3-position.

In a particular aspect, when A is (ii), X is CR⁴R⁸ and R⁸ is OH and more particularly OH is trans to R⁷. In a further aspect W¹ is a bond. In another aspect R⁷ is H. In an additional aspect W² and W³ are both CH₂. Where A is 3-hydroxypyrrolidin-4-ylmethyl, in a particular aspect the configuration is (3S,4S).

In a particular aspect, when A is (ii), X is O, R⁷ is H and W¹, W² and W³ are each CH₂.

In certain embodiments U is CH₂.

In certain embodiments R⁵ is an aromatic heterocyclic ring (B) having 8-11 ring atoms including 2-4 heteroatoms of which at least one is N or NR¹³ in which, in particular embodiments, Y² contains 2-3 heteroatoms, one of which is S and 1-2 are N, with one N bonded to X³.

In alternative embodiments the heterocyclic ring (B) has ring (a) aromatic selected from optionally substituted benzo, pyrido and pyridazino and ring (b) non aromatic and Y² has 3-4 atoms including at least one heteroatom, with O, S, CH₂ or NR¹³ bonded to X⁵, where R¹³ is other than hydrogen, and either NHCO bonded via N to X³, or O, S, CH₂, or NH bonded to X³. In a particular aspect the ring (a) contains aromatic nitrogen, and more particularly ring (a) is pyridine or pyrazine. Examples of rings (B) include optionally substituted:

### (a) and (b) Aromatic

1H-pyrrolo[2,3-b]-pyridin-2-yl, 1H-pyrrolo[3,2-b]-pyridin-2-yl, 3H-imidazo[4,5-b]-pyrid-2-yl, 3H-quinazolin-4-one-2-yl, benzimidazol-2-yl, benzo[1,2,3]-thiadiazol-5-yl, benzo[1,2,5]-oxadiazol-5-yl, benzofur-2-yl, benzothiazol-2-yl, benzo[b]thiophen-2-yl, benzoxazol-2-yl, chromen-4-one-3-yl, imidazo[1,2-a]pyridin-2-yl, imidazo-[1,2-a]-pyrimidin-2-yl, indol-2-yl, indol-6-yl, isoquinolin-3-yl, [1,8]-naphthyridine-3-yl, oxazolo[4,5-b]-pyridin-2-yl, quinolin-2-yl, quinolin-3-yl, quinoxalin-2-yl, naphthalen-2-yl, 1,3-dioxo-isoindol-2yl, 1H-benzotriazol-5-yl, 1H-indol-5-yl, 3H-benzooxazol-2-one-6-yl, 3H-benzooxazol-2-thione-6-yl, 3H-benzothiazol-2-one-5-yl, 3H-quinazolin-4-one-6-yl, pyrido[1,2-a]pyrimidin-4-one-3-yl, benzo[1,2,3]thiadiazol-6-yl, benzo[1,2,5]thiadiazol-5-yl, benzo[1,4]oxazin-2-one-3-yl, benzothiazol-5-yl, benzothiazol-6-yl, cinnolin-3-yl, imidazo[1,2-b]pyridazin-2-yl, pyrazolo[1,5-a]pyrazin-2-yl, pyrazolo[1,5-a]pyridin-2-yl, pyrazolo[1,5-a]pyrimidin-6-yl, pyrazolo[5,1-c][1,2,4]triazin-3-yl, pyrido[1,2-a]pyrimdin-4-one-2-yl, quinazolin-2-yl, quinoxalin-6-yl, thiazolo[3,2-a]pyrimidin-5-one-7-yl, thiazolo[5,4-b]pyridin-2-yl, thieno[3,2-b]pyridin-6-yl, thiazolo[5,4-b]pyridin-6-yl, thiazolo[4,5-b]pyridin-5-yl, [1,2,3]thiadiazolo[5,4-b]pyridin-6-yl, 2H-isoquinolin-1-one-3-yl

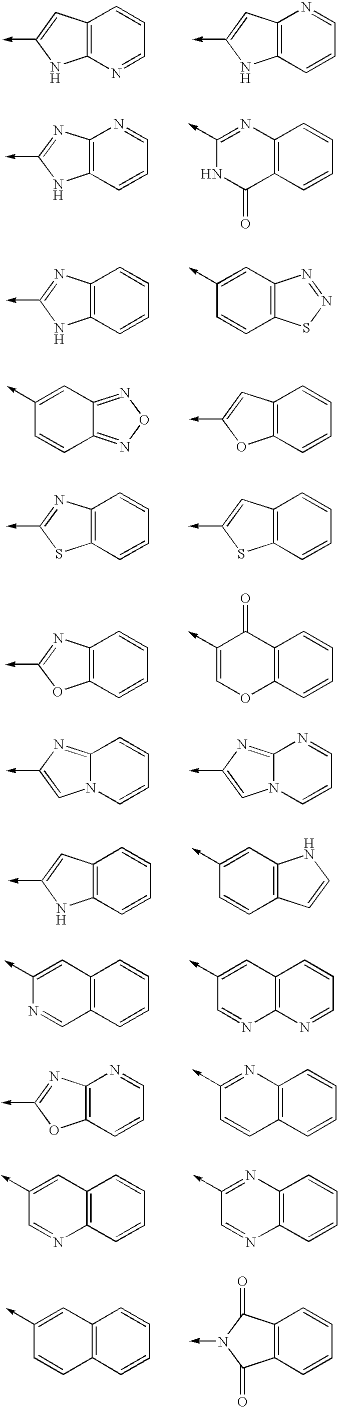

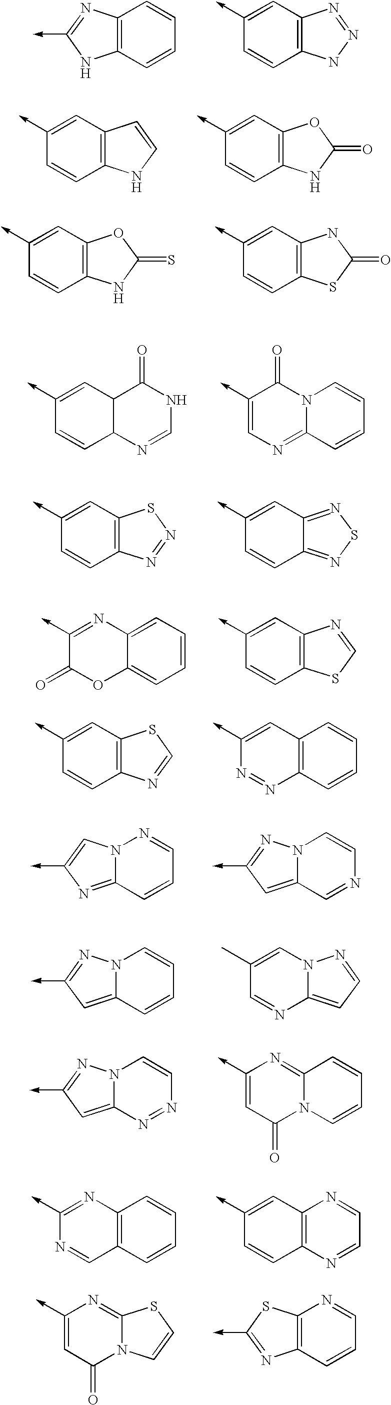

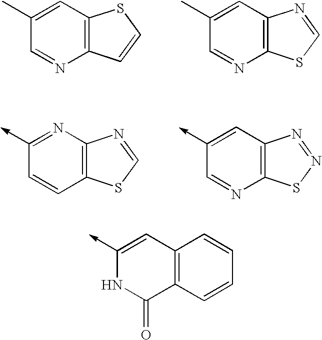

### a) is Non Aromatic

(2S)-2,3-dihydro-1H-indol-2-yl, (2S)-2,3-dihydro-benzo[1,4]dioxine-2-yl, 3-(R,S)-3,4-dihydro-2H-benzo[1,4]thiazin-3-yl, 3-(R)-2,3-dihydro-[1,4]dioxino[2,3-b]pyridin-3-yl, 3-(S)-2,3-dihydro-[1,4]dioxino[2,3-b]pyridin-3-yl, 2,3-dihydro-benzo[1,4]dioxan-2-yl, 3-substituted-3H-quinazolin-4-one-2-yl,

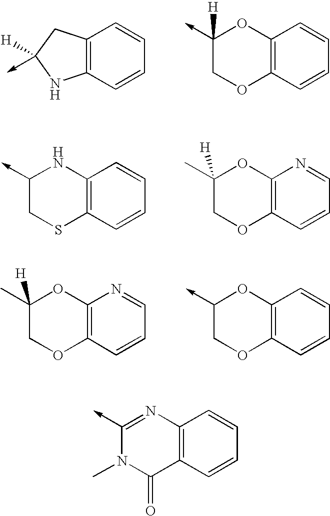

### (b) is Non Aromatic

1,1,3-trioxo-1,2,3,4-tetrahydrol l⁶-benzo[1,4]thiazin-6-yl, benzo[1,3]dioxol-5-yl, 2,3-dihydro-benzo[1,4]dioxin-6-yl, 2-oxo-2,3-dihydro-benzooxazol-6-yl, 3-substituted-3H-benzooxazol-2-one-6-yl, 3-substituted-3H-benzooxazole-2-thione-6-yl, 3-substituted-3H-benzothiazol-2-one-6-yl, 4H-benzo[1,4]oxazin-3-one-6-yl (3-oxo-3,4-dihydro-2H-benzo[1,4]oxazin-6-yl), 4H-benzo[1,4]thiazin-3-one-6-yl (3-oxo-3,4-dihydro-2H-benzo[1,4]thiazin-6-yl), 4H-benzo[1,4]oxazin-3-one-7-yl, 4-oxo-2,3,4,5-tetrahydro-benzo[b][1,4]thiazepine-7-yl, 5-oxo-2,3-dihydro-5H-thiazolo[3,2-a]pyrimidin-6-yl, 1H-pyrido[2,3-b][1,4]thiazin-2-one-7-yl(2-oxo-2,3-dihydro-1H-pyrido[2,3-b][1,4]thiazin-7-yl), 2,3-dihydro-1H-pyrido[2,3-b][1,4]thiazin-7-yl, 2-oxo-2,3-dihydro-1H-pyrido[3,4-b]thiazin-7-yl, 2,3-dihydro-[1,4]dioxino[2,3-b]pyridin-6-yl, 2,3-dihydro-[1,4]dioxino[2,3-c]pyridin-7-yl, 2,3-dihydro-[1,4]dioxino[2,3-b]pyridin-7-yl, 3,4-dihydro-2H-benzo[1,4]oxazin-6-yl, 3,4-dihydro-2H-benzo[1,4]thiazin-6-yl, 3-oxo-3,4-dihydro-2H-pyrido[3,2-b][1,4]oxazin-6-yl, 3,4-dihydro-2H-pyrido[3,2-b][1,4]thiazin-6-yl, 3-oxo-3,4-dihydro-2H-pyrido[3,2-b][1,4]thiazin-6-yl, 3,4-dihydro-1H-quinolin-2-one-7-yl, 3,4-dihydro-1H-quinoxalin-2-one-7-yl, 6,7-dihydro-4H-pyrazolo[1,5-a]pyrimidin-5-one-2-yl, 5,6,7,8-tetrahydro-[1,8]naphthyridin-2-yl, 2-oxo-3,4-dihydro-1H-[1,8]naphthyridin-6-yl, 6-oxo-6,7-dihydro-5H-8-thia-1,2,5-triaza-naphthalen-3-yl, 2-oxo-2,3-dihydro-1H-pyrido[3,4-b][1,4]oxazin-7-yl, 2-oxo-2,3-dihydro-1H-pyrido[2,3-b][1,4]oxazin-7-yl, 6,7-dihydro-[1,4]dioxino[2,3-d]pyrimidin-2-yl, [1,3]oxathiolo[5,4-c]pyridin-6-yl, 3,4-dihydro-2H-pyrano[2,3-c]pyridine-6-yl, 2,3-dihydro[1,4]oxathiino[2,3-c]pyridine-7-yl, 2,3-dihydrofuro[2,3-c]pyridin-5-yl, 2,3-dihydro-1-benzofuran-5-yl, indan-2-yl, 6,7-dihydro[1,4]dioxino[2,3-c]pyridazin-3-yl.

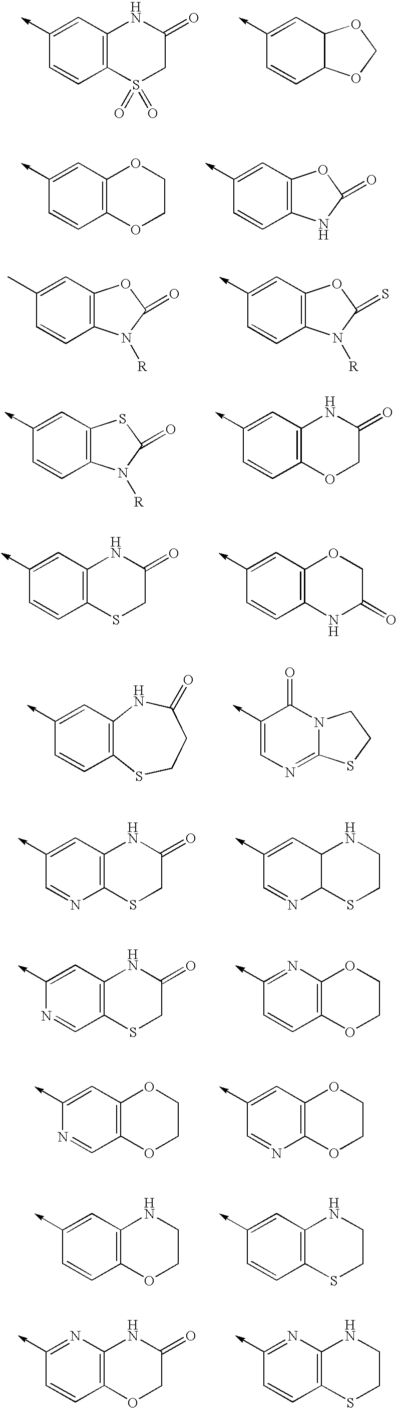

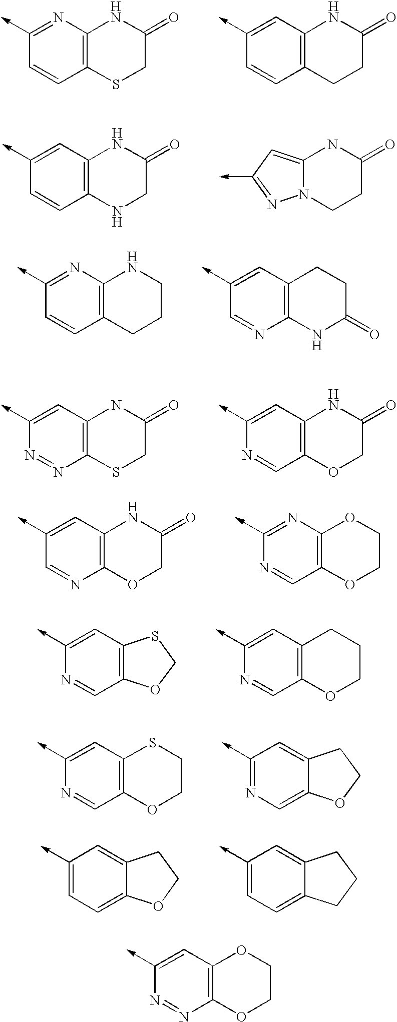

In some embodiments R¹³ is H if in ring (a) or in addition (C₁₋₄)alkyl such as methyl or isopropyl when in ring (b). More particularly, in ring (b) R¹³ is H when NR¹³ is bonded to X³ and (C₁₋₄)alkyl when NR¹³ is bonded to X⁵.

In further embodiments R¹⁴ and R¹⁵ are independently selected from hydrogen, halo, hydroxy, (C₁₋₄) alkyl, (C₁₋₄)alkoxy, nitro and cyano. More particularly R¹⁵ is hydrogen.

More particularly each R¹⁴ is selected from hydrogen, chloro, fluoro, hydroxy, methyl, methoxy, nitro and cyano. Still more particularly R¹⁴ is selected from hydrogen, fluorine or nitro.

Most particularly R¹⁴ and R¹⁵ are each H.

Particular groups R⁵ include:
[1,2,3]thiadiazolo[5,4-b]pyridin-6-yl
1H-pyrrolo[2,3-b]pyridin-2-yl
2,3-dihydro-[1,4]dioxino[2,3-b]pyridin-6-yl
2,3-dihydro-[1,4]dioxino[2,3-b]pyridin-7-yl
2,3-dihydro-[1,4]dioxino[2,3-c]pyridin-7-yl
2,3-dihydro-benzo[1,4]dioxin-6-yl
2-oxo-2,3-dihydro-1H-pyrido[2,3-b][1,4]oxazin-7-yl
2-oxo-2,3-dihydro-1H-pyrido[2,3-b][1,4]thiazin-7-yl
3,4-dihydro-2H-benzo[1,4]oxazin-6-yl
3-methyl-2-oxo-2,3-dihydro-benzooxazol-6-yl
3-oxo-3,4-dihydro-2H-benzo[1,4]oxazin-6-yl
3-oxo-3,4-dihydro-2H-pyrido[3,2-b][1,4]oxazin-6-yl (6-substituted 2H-pyrido[3,2-b][1,4]oxazin-3(4N)-one)
3-oxo-3,4-dihydro-2H-benzo[1,4]thiazin-6-yl(4H-benzo[1,4]thiazin-3-one-6-yl)
4-oxo-4H-pyrido[1,2-a]pyrimidin-2-yl
6-nitro-benzo[1,3]dioxol-5-yl
7-fluoro-3-oxo-3,4-dihydro-2H-benzo[1,4]oxazin-6-yl
8-hydroxy-1-oxo-1,2-dihydro-isoquinolin-3-yl
8-hydroxyquinolin-2-yl
benzo[1,2,3]thiadiazol-5-yl
benzo[1,2,5]thiadiazol-5-yl
benzothiazol-5-yl
thiazolo-[5,4-b]pyridin-6-yl
3-oxo-3,4-dihydro-2H-pyrido[3,2-b][1,4]thiazin-6-yl (6-substituted 2H-pyrido[3,2-b][1,4]thiazin-3(4H)-one)
7-chloro-3-oxo-3,4-dihydro-2H-pyrido[3,2-b][1,4]thiazin-6-yl
7-chloro-3-oxo-3,4-dihydro-2H-pyrido[3,2-b][1,4]oxazin-6-yl (6-substituted 7-chloro-2H-pyrido[3,2-b][1,4]oxazin-3(4H)-one)
7-fluoro-3-oxo-3,4-dihydro-2H-pyrido[3,2-b][1,4]thiazin-6-yl
2-oxo-2,3-dihydro-1H-pyrido[3,4-b][1,4]thiazin-7-yl
[1,3]oxathiolo[5,4-c]pyridin-6-yl
3,4-dihydro-2H-pyrano[2,3-c]pyridine-6-yl
2,3-dihydro-5-carbonitro-1,4-benzodioxin-7-yl (7-substituted 2,3-dihydro-1,4-benzodioxin-5-carbonitrile)
2,3-dihydro[1,4]oxathiino[2,3-c]pyridine-7-yl
2,3-dihydrofuro[2,3-c]pyridin-5-yl
5-fluoro-2,3-dihydro-1,4-benzodioxino-7-yl
2,3-dihydro-1-benzofuran-5-yl
7-cyano-2,3-dihydro-1-benzofuran-5-yl (5-substituted 2,3-dihydro-1-benzofuran-7-carbonitrile)
6,7-dihydro[1,4]dioxino[2,3-c]pyridazin-3-yl

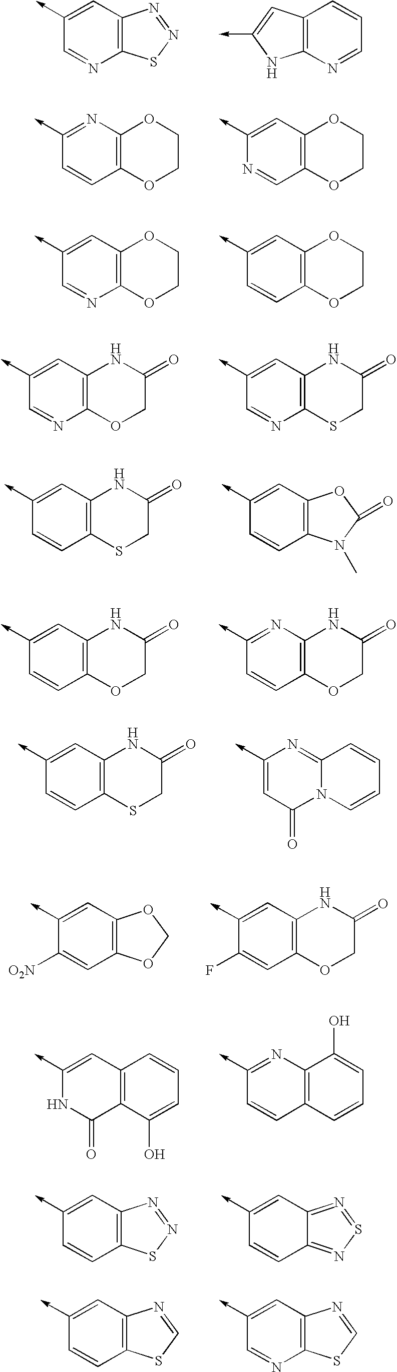

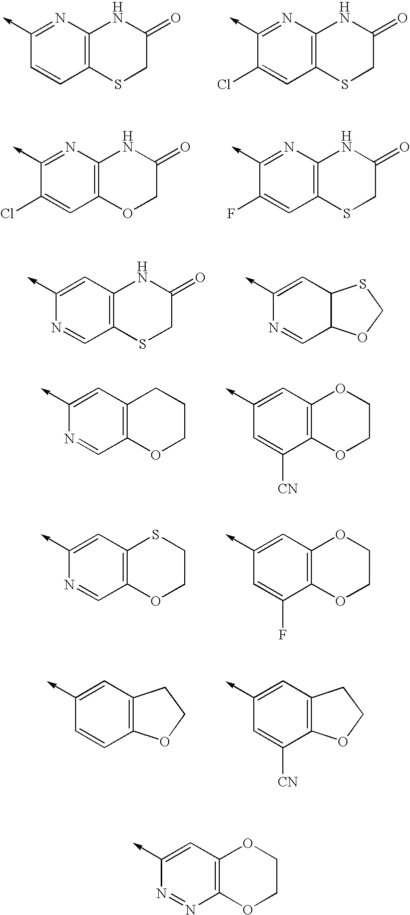

especially
6-substituted 2H-pyrido[3,2-b][1,4]oxazin-3(4H)-one
2,3-dihydro-[1,4]dioxino[2,3-c]pyridin-7-yl
[1,3]oxathiolo[5,4-c]pyridin-6-yl
3,4-dihydro-2H-pyrano[2,3-c]pyridine-6-yl
6-substituted 2H-pyrido[3,2-b][1,4]thiazin-3(4H)-one
6-substituted 7-chloro-2H-pyrido[3,2-b][1,4]oxazin-3(4H)-one
6,7-dihydro[1,4]dioxino[2,3-c]pyridazin-3-yl

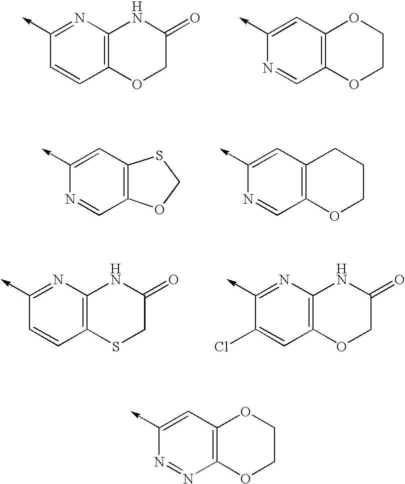

When used herein, the term “alkyl” includes groups having straight and branched chains, for instance, methyl, ethyl, n-propyl, iso-propyl, n-butyl, iso-butyl, sec-butyl, t-butyl, pentyl and hexyl. The term ‘alkenyl’ should be interpreted accordingly.

Halo or halogen includes fluoro, chloro, bromo and iodo.

Haloalkyl moieties include 1-3 halogen atoms.

Compounds within the invention contain a heterocyclyl group and may occur in two or more tautomeric forms depending on the nature of the heterocyclyl group; all such tautomeric forms are included within the scope of the invention.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Furthermore, it will be understood that phrases such as “a compound of formula (I) or a pharmaceutically acceptable salt, solvate or N-oxide thereof” are intended to encompass the compound of formula (I), an N-oxide of formula (I), a pharmaceutically acceptable salt of the compound of formula (I), a solvate of formula (I), or any pharmaceutically acceptable combination of these. Thus by way of non-limiting example used here for illustrative purpose, “a compound of formula (I) or a pharmaceutically acceptable salt or solvate thereof” may include a pharmaceutically acceptable salt of a compound of formula (I) that is further present as a solvate.

Since the compounds of formula (I) are intended for use in pharmaceutical compositions it will readily be understood that in particular embodiments they are provided in substantially pure form, for example at least 60% pure, more suitably at least 75% pure and particularly at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and more particularly from 10 to 59% of a compound of the formula (I) or pharmaceutically acceptable salt, solvate or N-oxide.

Particular compounds according to the invention include those mentioned in the examples and their pharmaceutically acceptable N-oxides, salts and solvates.

Pharmaceutically acceptable salts of the above-mentioned compounds of formula (I) include the acid addition or quaternary ammonium salts, for example their salts with mineral acids e.g. hydrochloric, hydrobromic, sulphuric nitric or phosphoric acids, or organic acids, e.g. acetic, fumaric, succinic, maleic, citric, benzoic, p-toluenesulphonic, methanesulphonic, naphthalenesulphonic acid or tartaric acids. Compounds of formula (I) may also be prepared as the N-oxide. The invention extends to all such derivatives.

Certain of the compounds of formula (I) may exist in the form of optical isomers, e.g. diastereoisomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

In a further aspect of the invention there is provided a process for preparing compounds of formula (I), and pharmaceutically acceptable salts, solvates and/or N-oxides thereof, which process comprises reacting a compound of formula (II) with a compound of formula (III):

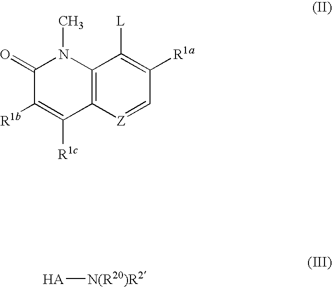

R²⁰ is UR⁵ or a group convertible thereto, L is —CH₂—CHO or —CH₂═CH₂ and R^{2′} is R² or a group convertible thereto, wherein Z, A, R^{1a}, R^{1b}, R², U and R⁵ are as defined in formula (I), and and thereafter optionally or as necessary converting R²⁰ and R^{2′} to UR⁵ and R², interconverting any variable groups, and/or forming a pharmaceutically acceptable salt, solvate or N-oxide thereof.

Where L is —CH₂—CHO the reaction is a reductive alkylation with a suitable reducing agent such as sodium cyanoborohydride (in methanol/chloroform/acetic acid) or triacetoxyborohydride. If the amine is present as a hydrochloride salt it is preferable to have an excess of sodium acetate present to buffer the reaction. 3A Molecular sieves may also be used to help formation of the initial imine intermediate.

Where L is —CH₂═CH₂ the reaction is a conjugate addition (Michael reaction) which involves direct reaction with the amine (III). The process is preferably carried out in a polar organic solvent e.g. acetonitrile, dimethylformamide or chloroform optionally in the presence of an organic base e.g. tetramethylguanidine (TMG) or triethylamine. In some cases an elevated temperature such as 40-150° C. may be beneficial.

Conveniently one of R²⁰ and R^{2′} is an N-protecting group, such as such as t-butoxycarbonyl, benzyloxycarbonyl, 9-fluorenylmethyloxycarbonyl or trifluoroacetyl. This may be removed by several methods well known to those skilled in the art (for examples see “Protective Groups in Organic Synthesis, T. W. Greene and P. G. M. Wuts, Wiley-Interscience, 1999), for example conventional acid hydrolysis (e.g. trifluoroacetic acid/dichloromethane, hydrochloric acid/dichloromethane/methanol), or potassium carbonate/methanol and the free amine converted to NR²UR⁵ by conventional means such as amide formation with an acyl derivative R⁵COW, for compounds where U is CO or, where U is CH₂, by alkylation with an alkyl halide R⁵CH₂-halide in the presence of base, acylation/reduction with an acyl derivative R⁵COW or reductive alkylation with an aldehyde R⁵CHO under conventional conditions (see for examples Smith, M. B.; March, J. M. Advanced Organic Chemistry, Wiley-Interscience). Suitable conditions include sodium cyanoborohydride (in methanol/chloroform/acetic acid). If the amine (III) is a hydrochloride salt then sodium acetate may be added to buffer the reaction. Sodium triacetoxyborohydride or (polystyrylmethyl)trimethylammonium cyanoborohydride are alternative reducing agents.

The appropriate reagents containing the required R⁵ group are known compounds or may be prepared analogously to known compounds, see for example WO02/08224, WO02/50061, WO02/56882, WO02/96907, WO2003087098, WO2003010138, WO2003064421, WO2003064431, WO2004002992, WO2004002490, WO2004014361, WO2004041210,WO2004096982, WO2002050036, WO2004058144, WO2004087145, WO2006014580, WO2004/035569, WO2004/089947, WO2003082835, WO2002026723, WO06002047, WO06014580, WO06010040, WO06017326, WO06012396, WO06017468, WO06020561 and EP0559285.

The compound of formula (II) where L is —CH₂—CHO may be prepared by the following Scheme 1:

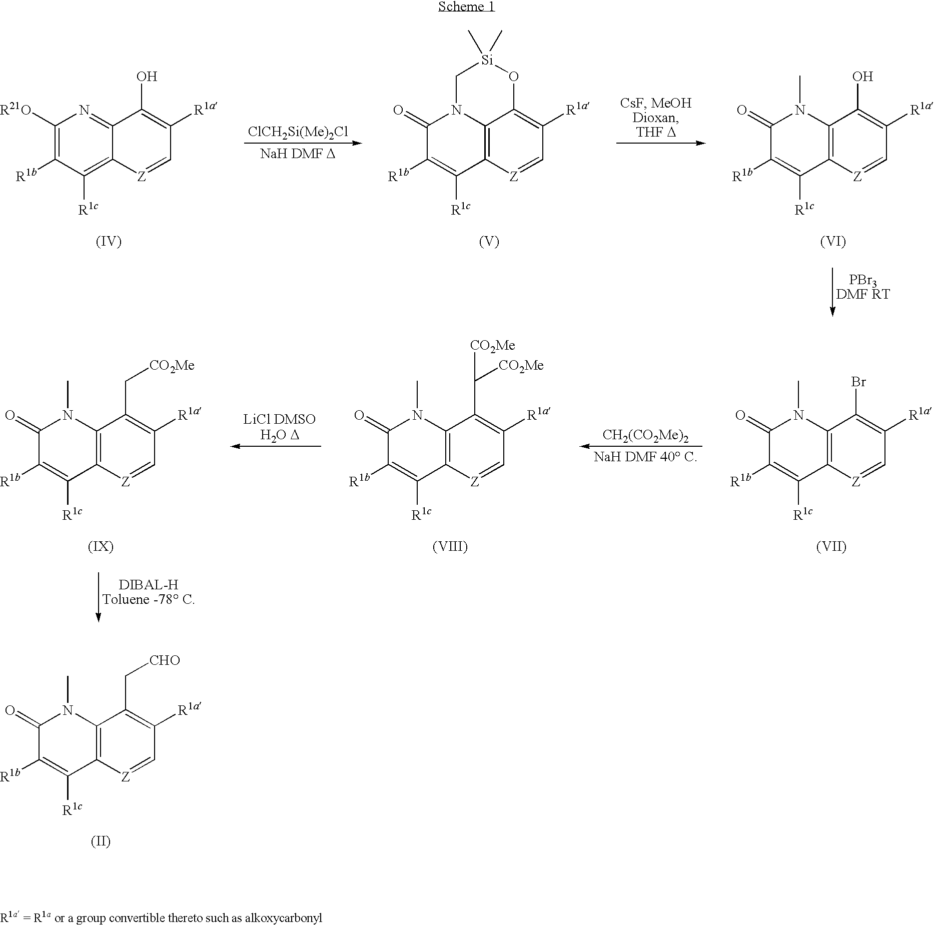

The 4-hydroxy derivative (IV), is reacted with sodium hydride, then chloro-(chloromethyl)dimethylsilane to give the cyclic silane (V), which is de-silylated with caesium fluoride to give the N-methyl-derivative (VI). This is converted to the 4-bromo derivative (VII) by reaction with PBr₃, (a 4-trifluoromethanesulfonate substituent is also possible via trifluoromethanesulfonic anhydride) and reacted with sodium malonate in DMF at 40° C. to give the malonate (VIII). This is decarboxylated (lithium chloride in wet DMSO) to give the mono-ester (IX), which is reduced at −78° C. with diisobutylaluminium hydride in toluene to the aldehyde intermediate (II).

The compound of formula (II) where L is —CH₂═CH₂ may be prepared by a similar route to Scheme 1 except that the 4-bromo-derivative (VII) is converted to the 4-vinyl compound (II) by conventional procedures such as a Suzuki reaction via trivinylcyclotriboroxane ((J. Org. Chem. 2002, 67, 4968-4971). Scheme 2 illustrates use of trivinylcyclotriboroxane pyridine complex (triethenylboroxin pyridine complex or vinylboroxine) and tetrakis(triphenylphosphine)palladium (0) as catalyst and potassium carbonate as the base

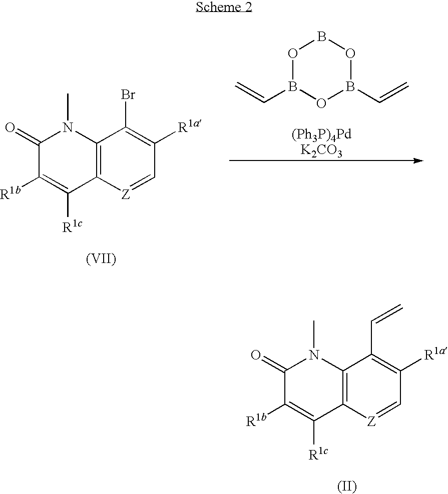

The compound of formula (II) where L is —CH₂—CHO may also be prepared by the following Scheme 3:

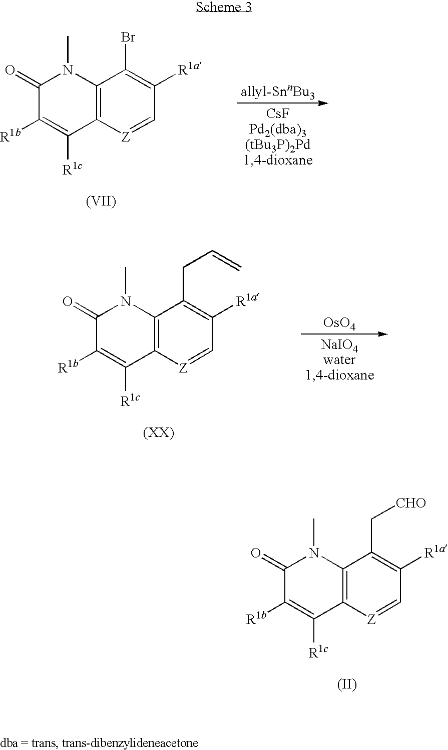

The 4-bromo-derivative (VII) is converted to the 4-allyl compound (XX) by conventional procedures such as a Stille reaction with allyltributylstannane in the presence of a Palladium catalyst/phosphine ligand combination (for example tris(dibenzylideneacetone)dipalladium(0) and bis(tri-tert-butylphosphine)palladium(0), J. Am. Chem. Soc., 2002, 124, 6343) in a suitable solvent such as 1,4-dioxane. The allyl compound (X) may then undergo oxidative cleavage by conventional methods, such as treatment with osmium tetroxide and sodium periodate, to give the aldehyde (II).

The compound of formula (II) where L is —CH₂═CH₂ may be converted to the compound where L is —CH₂—CHO by the following Scheme 4:

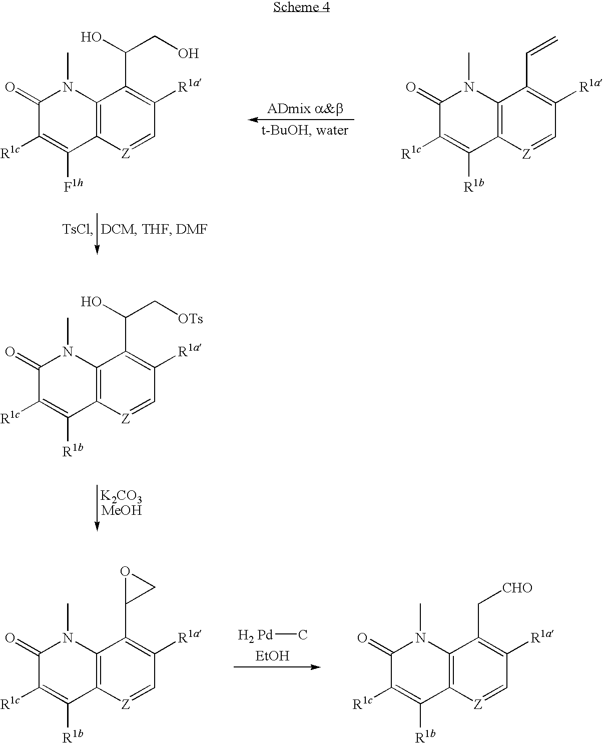

Conversions of R^{1a′} to R^{1a} and interconversions of R^{1a}, R^{1b}, R², A and R⁵ are conventional. For example R^{1a′} alkoxycarbonyl may be converted to R^{1a} carboxy by hydrolysis, which in turn may be converted to R^{1a} aminocarbonyl and cyano by conventional procedures. R^{1a} halo may be introduced by conventional halogenation reactions eg chlorination with chlorosuccinimide in acetic acid to introduce a chloro group at R^{1b}. In compounds which contain an optionally protected hydroxy group, suitable conventional hydroxy protecting groups which may be removed without disrupting the remainder of the molecule include acyl and alkylsilyl groups. N-protecting groups are removed by conventional methods.

For example R^{1a} or R^{1b} methoxy is convertible to R^{1a} or R^{1b} hydroxy by treatment with lithium and diphenylphosphine (general method described in Ireland et al, J. Amer. Chem. Soc., 1973, 7829) or HBr. Alkylation of the hydroxy group with a suitable alkyl derivative bearing a leaving group such as halide, yields R^{1a} or R^{1b} substituted alkoxy. R^{1a} halogen is convertible to other R^{1a} by conventional means, for example to hydroxy, alkylthiol (via thiol) and amino using metal catalysed coupling reactions, for example using copper as reviewed in Synlett (2003), 15, 2428-2439 and Angewandte Chemie, International Edition, 2003, 42(44), 5400-5449. R^{1a} fluoro may be converted to methoxy by treatment with sodium methoxide in methanol.

Compounds of formula HA-N(R²⁰)R^{2′} and (IV) are known compounds or may be prepared analogously to known compounds, see for example WO2004/035569, WO2004/089947, WO02/08224, WO02/50061, WO02/56882, WO02/96907, WO2003087098, WO2003010138, WO2003064421, WO2003064431, WO2004002992, WO2004002490, WO2004014361, WO2004041210,WO2004096982, WO2002050036, WO2004058144, WO2004087145, WO2003082835, WO2002026723, WO06002047 and WO06014580.

As shown in Scheme 5, the hydroxy-aminomethylpyrrolidines of formula (III) (A is (ii), X is CR⁴R⁸, W¹ is a bond, W² and W³ are both CH₂, R⁴ and R⁷ are H and R⁸ is OH) can be prepared from doubly protected chiral intermediate (X), separated by preparative HPLC. The benzyloxycarbonyl protecting group is removed by hydrogenation to give (XI) and the amino function converted to a trifluoroacetamide (XII). The t-butoxycarbonyl (Boc) protecting group is removed with HCl to give the pyrrolidine hydrochloride salt (III).

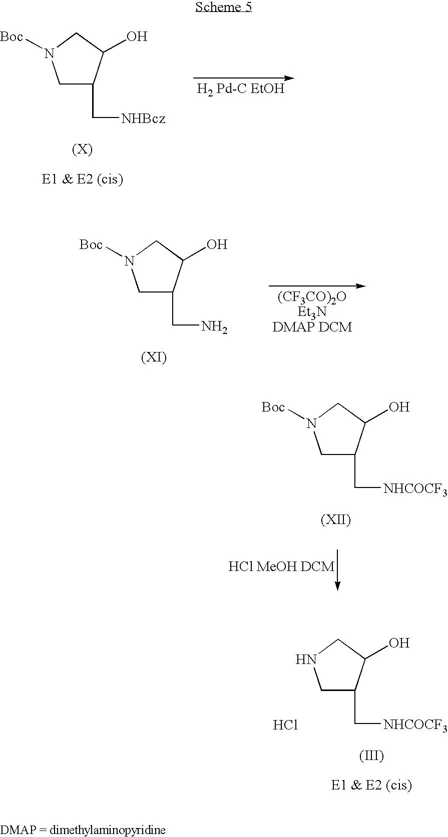

The intermediate (X) may be prepared by the general method of Scheme 6:

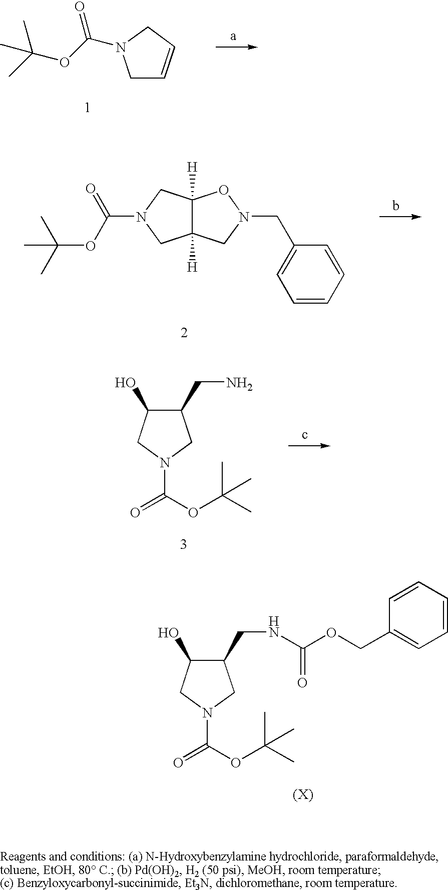

In Scheme 7 the aminomethylpyrrolidine of formula (III) (A is (ii), X is CR⁴R⁸, W¹ is a bond, W² and W³ are both CH₂, R⁴, R⁷ and R⁸ are all H) can be prepared from commercially available Boc-protected aminomethylpyrrolidine, and converted to the trifluoroacetamide.

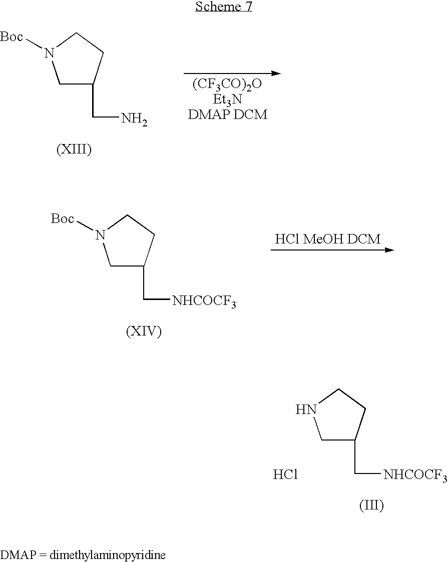

The aminomethylmorpholine intermediate of formula (III) (A is (ii), X is O, W¹, W² and W³ are each CH₂) may be prepared from a chiral dichlorobenzyl intermediate (XV) (WO2003082835) (Scheme 8) by first protecting the amino function with a Boc-protecting group (XVI), removing the dichlorobenzyl group by hydrogenation to give (III), protecting the morpholine N-atom with a benzyloxycarbonyl group (to allow purification by chromatography) (XVIII), and hydrogenation to afford the required morpholine derivative (III).

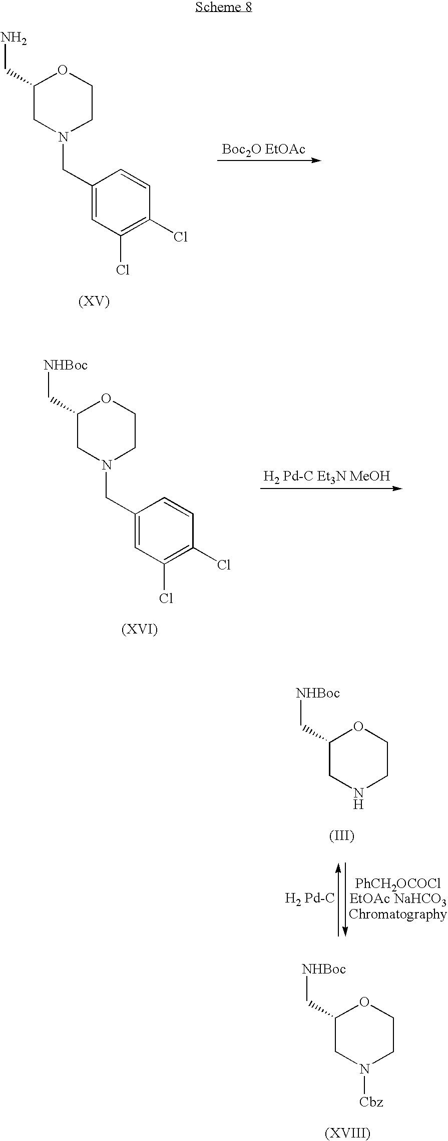

Further details for the preparation of compounds of formula (I) are found in the examples.

The antibacterial compounds according to the invention may be formulated for administration in any convenient way for use in human or veterinary medicine, by analogy with other antibacterials.

The pharmaceutical compositions of the invention include those in a form adapted for oral, topical or parenteral use and may be used for the treatment of bacterial infection in mammals including humans.

The composition may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-1000 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 30 mg/kg per day.

The compound of formula (I) may be the sole therapeutic agent in the compositions of the invention or a combination with other antibacterials. If the other antibacterial is a β-lactam then β-lactamase inhibitor may also be employed.

Compounds of formula (I) may be used in the treatment of bacterial infections caused by a wide range of organisms including both Gram-negative and Gram-positive organisms. Some compounds of formula (I) may be active against more than one organism. This may be determined by the methods described herein.

All publications, including but not limited to patents and patent applications, cited in this specification are herein incorporated by reference as if each individual publication were specifically and individually indicated to be incorporated by reference herein as though fully set forth.

The following examples illustrate the preparation of certain compounds of formula (I) and the activity of certain compounds of formula (I) against various bacterial organisms.

### EXAMPLES AND EXPERIMENTAL

### General

### Abbreviations in the Examples:

LCMS or LC-MS=Liquid chromatography mass spec.
HPLC=High Performance Liquid Chromatography
Mass directed autoprep=mass directed preparative HPLC (using a ZQ mass spectrometer (Waters))
Psi=pounds per square inch. 1 Psi=0.069 bar or 0.068 atmospheres

Certain reagents are also abbreviated herein. DMF refers to dimethylformamide, TFA refers to trifluoroacetic acid, THF refers to tetrahydrofuran, Et₃N refers to triethylamine, DCM refers to dichloromethane, Boc refers to tert-Butoxycarbonyl EtOH refers to ethanol.

Proton nuclear magnetic resonance (¹H NMR) spectra were recorded at 400, or 250 MHz, and chemical shifts are reported in parts per million (δ) downfield from the internal standard tetramethylsilane (TMS). Abbreviations for NMR data are as follows: s=singlet, d=doublet, t=triplet, q=quartet, m=multiplet, dd=doublet of doublets, dt=doublet of triplets, app=apparent, br=broad. CDCl₃ is deuteriochloroform, DMSO-d₆ is hexadeuteriodimethylsulfoxide, and CD₃OD is tetradeuteriomethanol. Mass spectra were obtained using electrospray (ES) ionization techniques. All temperatures are reported in degrees Celsius. E. MP-carbonate refers to macroporous triethylammonium methylpolystyrene carbonate (Argonaut Technologies).

Celite™ is a filter aid composed of acid-washed diatomaceous silica, and is a trademark of Manville Corp., Denver, Colo.

AD mix alpha is prepared by mixing potassium osmate (K₂OsO₄.2H₂O) (0.52 g), (3a,9R,3′″a,4′″b,9′″R)-9,9′-[1,4-phthalazinediylbis(oxy)]bis[6′-(methyloxy)-10,11-dihydrocinchonan] [(DHQ)₂PHAL] (5.52 g), then adding potassium ferricyanide [K₃Fe(CN)₆] (700 g) and powdered potassium carbonate (294 g). This mixture is stirred in a blender for 30 minutes. This provides approximately 1 kg of AD mix alpha, which is commercially available from Aldrich. See K. Barry Sharpless et al, J. Org. Chem., 1992, 57 (10), 2771. AD mix beta is the corresponding mixture prepared with (9S,9′″S)-9,9′-[1,4-phthalazinediylbis(oxy)]bis[6′-(methyloxy)-10,11-dihydrocinchonan] [(DHQD)2PHAL]. Where AD mix alpha/beta is referred to, this is a 1:1 mixture of the alpha and beta mix.

Reactions involving metal hydrides including lithium hydride, lithium aluminium hydride, di-isobutylaluminium hydride, sodium hydride, sodium borohydride, sodium triacetoxyborohydride, (polystyrylmethyl)trimethylammonium cyanoborohydride are carried out under argon.

As will be understood by the skilled chemist, references to preparations carried out according to or by the general method of other preparations, may encompass variations in routine parameters such as time, temperature, workup conditions, minor changes in reagent amounts, etc.

### Example 1

### 7-Chloro-6-[({1-[2-(3-chloro-5-methyl-6-oxo-5,6-dihydro-1,5-naphthyridin-4-yl)ethyl]-4-piperidinyl}amino)methyl]-2H-pyrido[3,2-b][1,4]oxazin-3(4H)-one dihydrochloride

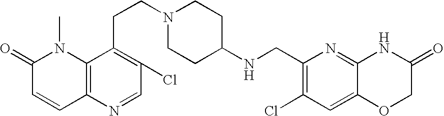

### (a) 3-Chloro-6-(methyloxy)-1,5-naphthyridin-4-ol

A solution of 6-(methyloxy)-1,5-naphthyridin-4-ol (6-methoxy-1H-[1,5]naphthyridin-4-one, for a synthesis see WO2002096907 Example 1) (50 g; 0.2841 mol) in glacial acetic acid (750 mL) was treated with N-chlorosuccinimide (42.5 g; 0.3185 mol) and the mixture was heated under argon at 35° C. for 24 hours. It was cooled and the precipitated solid filtered off and washed with a little cold acetic acid, then ether and finally hexane. It was dried at 50° C. overnight in vacuo to give an off-white solid 48 g (80%).

LC/MS (+ve ion electrospray): m/z 209/211 (M+H)⁺

### (b) 10-Chloro-2,2-dimethyl-2,3-dihydro-5H-[1,4,2]oxazasilino[6,5,4-de]-1,5-naphthyridin-5-one

A 60% suspension of sodium hydride in oil (6.0 g) was suspended in dry DMF (700 mL) under argon and 3-chloro-6-(methyloxy)-1,5-naphthyridin-4-ol (20 g; 0.09524 mol) was added portionwise to the stirred mixture [initial cooling in an ice-bath] and the mixture was allowed to warm to room temperature over 1 hour (all dissolved). Chloro(chloromethyl)dimethylsilane (24 mL; 0.01678 mol) was added dropwise over 10 minutes and the mixture was stirred at room temperature for 1.5 hours and then heated at 100° C. overnight. The reaction was shown to be complete by LC/MS (+ve ion electrospray): m/z 267/9 (M+H)⁺ (very broad peak). The mixture was evaporated to dryness, azeotroped with dry toluene, and chromatographed on a silica column (500 g), eluting with 0-12% methanol-DCM to give an off-white solid (24.5 g; 97%), which was dried in vacuo overnight.

### (c) 7-Chloro-8-hydroxy-1-methyl-1,5-naphthyridin-2(1H)-one

10-Chloro-2,2-dimethyl-2,3-dihydro-5H-[1,4,2]oxazasilino[6,5,4-de]-1,5-naphthyridin-5-one (36 g) in dry THF (400 mL), dioxan (400 mL) and methanol (500 mL) [dioxan was added to increase the reflux temperature] was treated with cesium fluoride (36 g) and the mixture was stirred and heated under reflux for 72 hours (monitored by LC/MS until all starting material consumed). It was evaporated to dryness, then water (5 mL)/methanol (100 mL) was added followed by 2M HCl, dropwise, to pH 3-4. The solid was collected, washed with a small volume of water, then methanol, and finally ether. It was dried at 50° C., in vacuo, overnight, to give an off-white solid (28.5 g, %).

LC/MS (+ve ion electrospray): m/z 211/213 (M+H)⁺

### (d) 8-Bromo-7-chloro-1-methyl-1,5-naphthyridin-2(1H)-one

A finely-ground suspension of 7-chloro-8-hydroxy-1-methyl-1,5-naphthyridin-2(1H)-one (20.6 g, 98.4mmol) in anhydrous dimethylformamide (1.0 L) was cooled in ice while phosphorus tribromide (14.7 mL, 154.7 mmol) was added slowly. Most of the starting-material dissolved during the addition, and a new precipitate formed soon afterwards. The mixture was stirred at room temperature for 3.5 hours, then evaporated. The residue was cooled in ice while treating cautiously with aqueous sodium bicarbonate until basic, and extracted with dichloromethane. The extracts were washed with water, dried and evaporated to give a solid, 24.04 g (89%).

LC/MS (+ve ion electrospray): m/z 273/275/277 (M+H)⁺

### (e) Dimethyl(3-chloro-5-methyl-6-oxo-5,6-dihydro-1,5-naphthyridin-4-yl)propanedioate

Sodium hydride (60% dispersion in oil; 3.52 g) was added carefully to a solution of dimethylmalonate (11.6 g; 0.0878 mol) in dry DMF (100 mL), under argon, initially cooled in an ice-bath. The mixture was stirred (with occasional sonication) for 1 hour at room temperature and then 8-bromo-7-chloro-1-methyl-1,5-naphthyridin-2(1H)-one (8.0 g; 0.0293 mol) was added and the mixture was heated at 45° C. for 17 hours, then evaporated to dryness. Water and dichloromethane were added, followed by 1M HCl, to pH 4-5, and the mixture was extracted with dichloromethane, dried, evaporated and chromatographed on silica gel (ethyl acetate-hexane) to give a solid (3.18 g).

LC/MS (+ve ion electrospray): m/z 325/7 (M+H)⁺

### (f) Methyl(3-chloro-5-methyl-6-oxo-5,6-dihydro-1,5-naphthyridin-4-yl)acetate

Dimethyl(3-chloro-5-methyl-6-oxo-5,6-dihydro-1,5-naphthyridin-4-yl)propanedioate (1.0 g) in dry DMSO (20 mL) containing water (0.06 mL) and lithium chloride (0.13 g) was heated at 90° C. for 2 hours, cooled, poured into iced-dilute sodium bicarbonate solution and extracted with dichloromethane. The organic fraction was washed with water, dried, and evaporated to give a yellow solid (0.8 g).

LC/MS (+ve ion electrospray): m/z 267/9 (M+H)⁺

### (g) (3-Chloro-5-methyl-6-oxo-5,6-dihydro-1,5-naphthyridin-4-yl)acetaldehyde

Methyl(3-chloro-5-methyl-6-oxo-5,6-dihydro-1,5-naphthyridin-4-yl)acetate (1.0 g) in dry toluene (100 mL) at −78° C., under argon, was treated, portionwise, with a 1M solution of di-isobutylaluminium hydride (3×3.75 mL) over 3 hours. After 3.5 hours the solution was quenched with excess sodium potassium tartrate in water and allowed to warm to room temperature. It was filtered (kieselguhr) and the filtrate extracted with toluene, then dichloromethane, dried and evaporated to give the crude (fairly unstable) aldehyde (ca. 60-65% pure by LC-MS and NMR (CDCl₃; CHO peak at ca. δ 10.00 ppm), pure enough for reaction in the next step.

LC/MS (+ve ion electrospray): m/z 237/9 (M+H)⁺

### (h) 1,1-Dimethylethyl{1-[2-(3-chloro-5-methyl-6-oxo-5,6-dihydro-1,5-naphthyridin-4-yl)ethyl]-4-piperidinyl}carbamate

Impure (3-chloro-5-methyl-6-oxo-5,6-dihydro-1,5-naphthyridin-4-yl)acetaldehyde (equivalent to approx. 0.40 g; 0.001695 mol of pure material) and 1,1-dimethylethyl 4-piperidinylcarbamate (0.60 g; 0.003 mol) were stirred in dry methanol (8 mL) and chloroform (8 mL) and acetic acid (20 drops) with 3 A molecular sieves at room temperature for 2 hours, then sodium cyanoborohydride (0.32 g; 0.0051 mol) was added and the mixture was stirred at room temperature for 18 hours. Aqueous sodium carbonate was added and the mixture was filtered, extracted with 10% methanol-dichloromethane, dried, evaporated and chromatographed on silica gel (methanol-dichloromethane) to give a solid (0.70 g).

LC/MS (+ve ion electrospray): m/z 421/3 (M+H)⁺

### (i) 8-[2-(4-Amino-1-piperidinyl)ethyl]-7-chloro-1-methyl-1,5-naphthyridin-2(1H)-one dihydrochloride

1,1-Dimethylethyl{1-[2-(3-chloro-5-methyl-6-oxo-5,6-dihydro-1,5-naphthyridin-4-yl)ethyl]-4-piperidinyl}carbamate (0.70 g) in methanol (10 mL) and dichloromethane (40 mL) was treated with 4M hydrogen chloride in dioxan (30 mL) and the solution was stirred at room temperature for 3 hours and evaporated to give a solid that was triturated with ether and dried to give a solid (0.69 g).

LC/MS (+ve ion electrospray): m/z 321/3 (M+H)⁺

### (j) Title Compound

8-[2-(4-Amino-1-piperidinyl)ethyl]-7-chloro-1-methyl-1,5-naphthyridin-2(1H)-one dihydrochloride (70 mg; equivalent to 60 mg of pure material) was dissolved in dry methanol (3 mL), chloroform (3 mL) and acetic acid (8 drops). Anhydrous sodium acetate (75 mg) was added followed by 7-chloro-3-oxo-3,4-dihydro-2H-pyrido[3,2-b][1,4]oxazine-6-carboxaldehyde (for a synthesis see WO2003064421, Example 15(c)) (32 mg) and excess 3 A molecular sieves. The mixture was stirred at room temperature for 2.5 hours then sodium cyanoborohydride (30 mg) was added and the mixture was stirred at room temperature for 3.5 hours. Aqueous sodium carbonate was added and the mixture extracted with 10% methanol-chloroform, dried (sodium sulphate), evaporated and subjected to mass-directed autoprep purification (M 516; eluent: acetonitrile-water-formic acid) followed by treatment with 4M hydrogen chloride in dioxan. The solution was evaporated to give the title compound (24 mg), after trituration with ether.

¹H NMR δ(CD₃OD-CDCl₃) 3.87 (3H, s), 6.91 (1H, d), 7.52 (1H, s), 7.93 (1H, d), 8.59 (1H, s). (compound very insoluble—many signals weak or obscured by solvent).

LC/MS (+ve ion electrospray): m/z 517/9 (M+H)⁺

### Example 2

### 7-Chloro-1-methyl-8-(2-{4-[([1,3]oxathiolo[5,4-c]pyridin-6-ylmethyl)amino]-1-piperidinyl}ethyl)-1,5-naphthyridin-2(1H)-one dihydrochloride

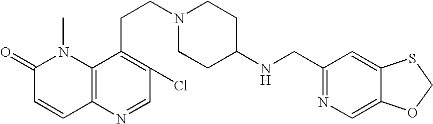

The title compound was prepared by reaction of 8-[2-(4-amino-1-piperidinyl)ethyl]-7-chloro-1-methyl-1,5-naphthyridin-2(1H)-one dihydrochloride (0.63 g; assume 0.6 g of pure material) and [1,3]oxathiolo[5,4-c]pyridine-6-carbaldehyde (0.255 g) (for a synthesis see WO2004058144, Example 61) by the general method of Example 1(j) (total reaction time 6.5 hr). The crude product after work-up was chromatographed on silica gel (0-20% methanol-DCM) to afford the free base.

¹H NMR δ(CDCl₃) 1.45-1.50 (2H, m), 1.89 (2H, d), 2.20 (3H, t), 2.52 (1H, m), 2.65 (2H, m), 2.93 (2H, m), 3.40 (2H, m), 3.83 (5H, s), 5.72 (2H, s), 6.85 (1H, d), 7.27 (1H, s), 7.82 (1H, d), 8.00 (1H, s), and 8.46 (1H, s).

The free base was treated with 4M hydrogen chloride in dioxan and the solution was evaporated to give the title compound (0.505 g), after trituration with ether.

LC/MS (+ve ion electrospray): m/z 472/4 (M+H)⁺

### Example 3

### 7-Chloro-8-[2-((3S,4S)-3-hydroxy-4-{[([1,3]oxathiolo[5,4-c]pyridin-6-ylmethyl)amino]methyl}-1-pyrrolidinyl)ethyl]-1-methyl-1,5-naphthyridin-2(1H)-one dihydrochloride

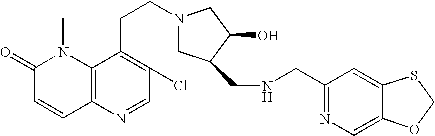

### (a) 1,1-Dimethylethyl(3S,4S)-3-(aminomethyl)-4-hydroxy-1-pyrrolidinecarboxylate

A solution of 1,1-dimethylethyl (3S,4S)-3-hydroxy-4-[({[(phenylmethyl)oxy]carbonyl}amino)methyl]-1-pyrrolidinecarboxylate (for a synthesis, see WO 2006/002047 Preparation 24(c), enantiomer E1) (2 g) in ethanol (30 mL) was hydrogenated over 10% palladium-carbon (1.0 g) for 18 hours. It was filtered and evaporated to give a foam (1.4 g).

LC/MS (+ve ion electrospray): m/z 217 (M+H)⁺

### (b) 1,1-Dimethylethyl(3S,4S)-3-hydroxy-4-{[(trifluoroacetyl)amino]methyl}-1-pyrrolidinecarboxylate

1,1-Dimethylethyl(3S,4S)-3-(aminomethyl)-4-hydroxy-1-pyrrolidinecarboxylate (1.40 g) in dry DCM (50 mL) was treated with triethylamine (1.8 mL) and 4-(dimethylamino)-pyridine (79 mg) followed by trifluoroacetic anhydride (0.915 mL), dropwise, and the solution was left stirring at room temperature overnight. Water was added and the solution was extracted with DCM, dried (sodium sulphate), evaporated, and chromatographed on silica gel (methanol-DCM) to afford a foam.

### (c) 2,2,2-Trifluoro-N-{[(3R,4S)-4-hydroxy-3-pyrrolidinyl]methyl}acetamide hydrochloride

1,1-Dimethylethyl(3S,4S)-3-hydroxy-4-{[(trifluoroacetyl)amino]methyl}-1-pyrrolidinecarboxylate in dry DCM (50 mL) was treated with 4M hydrogen chloride in dioxan (40 mL) and the mixture was stirred well for 4.5 hours and evaporated to give a solid (1.25 g).

LC/MS (+ve ion electrospray): m/z 213 (M+H)⁺

### (d) N-({(3S,4S)-1-[2-(3-Chloro-5-methyl-6-oxo-5,6-dihydro-1,5-naphthyridin-4-yl)ethyl]-4-hydroxy-3-pyrrolidinyl}methyl)-2,2,2-trifluoroacetamide

A mixture of impure (3-chloro-5-methyl-6-oxo-5,6-dihydro-1,5-naphthyridin-4-yl)acetaldehyde (equivalent to 0.10 g pure material), 2,2,2-trifluoro-N-{[(3R,4S)-4-hydroxy-3-pyrrolidinyl]methyl}acetamide hydrochloride (0.158 g), and anhydrous sodium acetate (0.26 g) in dry methanol (3 mL), chloroform (3 mL) and acetic acid (12 drops) and 3 A molecular sieves were stirred at room temperature for 2 hours, then sodium cyanoborohydride (0.08 g) was added and the mixture stirred at room temperature for 3 hours. Aqueous sodium carbonate was added and the mixture extracted with 10% methanol-chloroform, dried (sodium sulphate), evaporated and chromatographed on silica gel (methanol-DCM) to afford an oil (0.10 g).

LC/MS (+ve ion electrospray): m/z 433 (M+H)⁺

### (e) Title Compound

N-({(3S,4S)-1-[2-(3-Chloro-5-methyl-6-oxo-5,6-dihydro-1,5-naphthyridin-4-yl)ethyl]-4-hydroxy-3-pyrrolidinyl}methyl)-2,2,2-trifluoroacetamide (100 mg) in methanol (3 mL) and water (3 mL) was stirred with anhydrous potassium carbonate (128 mg) for 4 hours at room temperature, then evaporated and azeotroped with methanol, then toluene. Half of the residue was dissolved in methanol (2mL), chloroform (2 mL), and acetic acid (10 drops) and treated with anhydrous sodium acetate (67 mg), [1,3]oxathiazolo[5,4-c]pyridine-6-carbaldehyde (for a synthesis see WO2004058144, Example 61) (19.3 mg) and 3 A molecular sieves and stirred at room temperature for 2.5 hours. Sodium cyanoborohydride (44 mg) was added and the mixture was stirred at room temperature, overnight. Aqueous sodium carbonate was added and the mixture was extracted with 10% methanol-chloroform, dried (sodium sulphate), evaporated and subjected to mass-directed autoprep purification (M 487; eluent: acetonitrile-water-formic acid) followed by treatment with 4M hydrogen chloride in dioxan. The solution was evaporated to give the title compound (37 mg), after trituration with ether.

¹H NMR δ(CD₃OD) 3.40-3.80 (ca. 6H, m), 3.87 (3H, s), 3.95-4.15 (1H, m), 4.37 (2H, s), 4.65 (1H, m), 5.92 (2H, s), 6.92 (1H, d), 7.60 (1H, br. d), 7.92 (1H, d), 8.12 (1H, s) and 8.60 (1H, s). (several signals obscured by solvent).

LC/MS (+ve ion electrospray): m/z 488 (M+H)⁺

### Example 4

### 6-[({1-[2-(3-Chloro-5-methyl-6-oxo-5,6-dihydro-1,5-naphthyridin-4-yl)ethyl]-4-piperidinyl}amino)methyl]-2H-pyrido[3,2-b][1,4]thiazin-3(4H)-one dihydrochloride

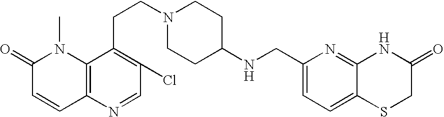

The title compound was prepared by the general method of Example 1(j) using 3-oxo-3,4-dihydro-2H-pyrido[3,2-b][1,4]thiazine-6-carboxaldehyde (for a synthesis see WO2003087098, Example 301(d)) as the aldehyde component, with a reaction time of 5 hours. The crude product was chromatographed on silica gel (methanol-DCM) to give the free base.

¹H NMR δ(CDCl₃) 1.45-1.50 (2H, m), 1.80 (1H, br. s), 1.89 (2H, d), 2.18 (2H, t), 2.52 (1H, m), 2.65 (2H, m), 2.93 (2H, d), 3.40 (2H, m), 3.45 (2H, s), 3.83 (5H, s), 6.87 (1H, d), 6.98 (1H, d), 7.57 (1H, d), 7.82 (1H, d), 8.50 (1H, s), and 8.65 (1H, br. s).

The free base was treated with 4M hydrogen chloride in dioxan and the solution was evaporated to give the title compound (35 mg), after trituration with ether.

LC/MS (+ve ion electrospray): m/z 499 (M+H)⁺

### Example 5

### 6-[({(3R,4S)-1-[2-(3-Chloro-5-methyl-6-oxo-5,6-dihydro-1,5-naphthyridin-4-yl)ethyl]-3-hydroxy-4-piperidinyl}amino)methyl]-2H-pyrido[3,2-b][1,4]oxazin-3(4H)-one dihydrochloride

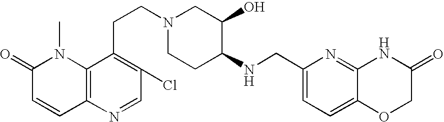

### (a) 1,1-Dimethylethyl{(3R,4S)-1-[2-(3-chloro-5-methyl-6-oxo-5,6-dihydro-1,5-naphthyridin-4-yl)ethyl]-3-hydroxy-4-piperidinyl}carbamate

(3-Chloro-5-methyl-6-oxo-5,6-dihydro-1,5-naphthyridin-4-yl)acetaldehyde (equivalent to ca. 0.11 g of pure material) and 1,1-dimethylethyl[(3R,4S)-3-hydroxy-4-piperidinyl]carbamate (for a synthesis see WO 2004/058144 Example 5(c) cis-(3-hydroxy-piperidin-4-yl)-carbamic acid tert-butyl ester enantiomer 1) (0.15 g) were stirred in dry methanol (2 mL), chloroform (2 mL) and acetic acid (10 drops) with 3 A molecular sieves at room temperature for 1 hour, then sodium cyanoborohydride (0.10 g) was added and the mixture was stirred at room temperature for 1.5 hours. Aqueous sodium carbonate was added and the mixture was filtered, extracted with 10% methanol-chloroform, dried (sodium sulphate), evaporated and chromatographed (twice) on silica gel (methanol-dichloromethane) to give a solid (0.21 g).

LC/MS (+ve ion electrospray): m/z 437/9 (M+H)⁺

### (b) 8-{2-[(3R,4S)-4-Amino-3-hydroxy-1-piperidinyl]ethyl}-7-chloro-1-methyl-1,5-naphthyridin-2(1H)-one

1,1-Dimethylethyl{(3R,45)-1-[2-(3-chloro-5-methyl-6-oxo-5,6-dihydro-1,5-naphthyridin-4-yl)ethyl]-3-hydroxy-4-piperidinyl}carbamate (0.21 g) in dry DCM (10 mL) was treated with trifluoroacetic acid (10 mL) for 2.5 hours at room temperature, evaporated, basified with sodium carbonate and extracted with 10% methanol-chloroform, dried (sodium sulphate), and evaporated to give an oil (0.17 g).

LC/MS (+ve ion electrospray): m/z 337/9 (M+H)⁺

### (c) Title Compound

The title compound was prepared by the general method of Example 1(j) from 8-{2-[(3R,4S)-4-amino-3-hydroxy-1-piperidinyl]ethyl}-7-chloro-1-methyl-1,5-naphthyridin-2(1H)-one (70 mg) and 3-oxo-3,4-dihydro-2H-pyrido[3,2-b][1,4]oxazine-6-carboxaldehyde (for a synthesis see WO2003087098, Example 31(e)) (37 mg) (total reaction time 5 hr). The crude product was chromatographed on silica gel (methanol-DCM) to give the free base.

¹H NMR δ(CDCl₃) 1.70-1.77 (2H, m), 2.20 (1H, m), 2.33 (1H, d), 2.60 (1H, m), 2.72 (2H, m), 2.89 (1H, br.d), 3.06 (1H, br.d), 3.40 (2H, m), 3.45 (2H, s), 3.80 (3H, s), 3.87 (2H, d), 3.90 (1H, s), 4.61 (2H, s), 6.87 (1H, d), 6.93 (1H, d), 7.19 (1H, d), 7.82 (1H, d), 8.46 (1H, s).

The free base was treated with 4M hydrogen chloride in dioxan and the solution was evaporated to give the title compound (46 mg), after trituration with ether.

LC/MS (+ve ion electrospray): m/z 499/501 (M+H)⁺

### Example 6

### 7-Chloro-1-methyl-8-[2-((2S)-2-{[([(1,3]oxathiolo[5,4-c]pyridin-6-ylmethyl)amino]methyl}-4-morpholinyl)ethyl]-1,5-naphthyridin-2(1H)-one dihydrochloride

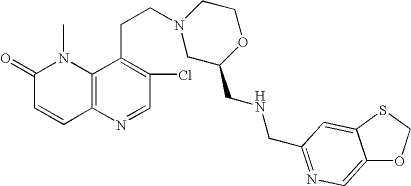

### (a) 1,1-Dimethylethyl({(2S)-4-[(3,4-dichlorophenyl)methyl]-2-morpholinyl}methyl)carbamate

({(2S)-4-[(3,4-Dichlorophenyl)methyl]-2-morpholinyl}methyl)amine (for a synthesis see WO03/082835A1 Example 1 [(2S)-4-(3,4-dichlorobenzyl)morpholin-2-yl]methylamine prepared using Burkholderia cepacia lipase immobilised on porous ceramic particles, available from Sigma-Aldrich Co. as Amano Lipase PS-C II, cat. No. 534889) (4.9 g) in ethyl acetate (40 mL) was stirred with di-tert-butyl dicarbonate (5.95 g) overnight at room temperature then evaporated and chromatographed on silica gel (0-5% methanol-DCM) to give the product (6.05 g).

### (b) Phenylmethyl(2S)-2-[({[(1,1-dimethylethyl)oxy]carbonyl}amino)methyl]-4-morpholinecarboxylate

1,1-Dimethylethyl({(2S)-4-[(3,4-dichlorophenyl)methyl]-2-morpholinyl}methyl)carbamate (2.0 g) in methanol (30 mL) with triethylamine (2.2 mL) was hydrogenated over 10% palladium on charcoal (1.0 g) at 50 psi for 24 hours then filtered through Celite™ and evaporated. The residue was stirred in ethyl acetate (50 mL), and saturated sodium bicarbonate (50 mL) with benzyl chloroformate (1.62 mL) overnight. The organic layer was separated, dried (sodium sulfate) and evaporated. Purification on silica gel (0-2% methanol-DCM) gave the product (1.3 g).

### (c) 1,1-Dimethylethyl[(2S)-2-morpholinylmethyl]carbamate

Phenylmethyl(2S)-2-[({[(1,1-dimethylethyl)oxy]carbonyl}amino)methyl]-4-morpholinecarboxylate (1.3 g) was dissolved in methanol (75 mL) and hydrogenated over 10% palladium on charcoal (0.5 g) overnight, then filtered through Celite™ and evaporated to give the amine (0.81 g).

¹H NMR δ (CDCl₃) 1.44 (9H, s), 2.50-2.65 (1H, m), 2.70-2.95 (3H, m), 2.95-3.10 (1H, m), 3.45-3.70 (2H, m), 3.80-3.90 (1H, m), 4.88 (1H, br s).

### (d) 1,1-Dimethylethyl({(2S)-4-[2-(3-chloro-5-methyl-6-oxo-5,6-dihydro-1,5-naphthyridin-4-yl)ethyl]-2-morpholinyl}methyl)carbamate

1,1-Dimethylethyl[(2S)-2-morpholinylmethyl]carbamate_(0.07 g), and (3-chloro-5-methyl-6-oxo-5,6-dihydro-1,5-naphthyridin-4-yl)acetaldehyde (0.25 g, 20% pure), sodium acetate (0.19 g), acetic acid (12 drops) in methanol (4 mL) and chloroform (4 mL) were stirred with 3 A sieves for 2 hours at room temperature. Sodium cyanoborohydride (45 mg) was added and the mixture stirred at room temperature for 3 hours then basified with sodium carbonate and extracted with 10% methanol in dichloromethane. The extracts were dried (sodium sulphate), evaporated and purified on silica gel (0-5% methanol-DCM) to give an oil (0.113 g).

LC/MS (+ve ion electrospray): m/z 336 (M−Bu+H)⁺, 459 (M+Na)⁺.

### (e) Title Compound

1,1-Dimethylethyl({(2S)-4-[2-(3-chloro-5-methyl-6-oxo-5,6-dihydro-1,5-naphthyridin-4-yl)ethyl]-2-morpholinyl}methyl)carbamate_(0.113 g) in methanol (0.5 mL) and dichloromethane (0.5 mL) was stirred with 4M hydrogen chloride in dioxan (0.2 mL) for 1 hours, further 4M hydrogen chloride in dioxan added (1.0 ml) and stirring continued for 2 h, then evaporated. The resulting amine hydrochloride, [1,3]oxathiolo[5,4-c]pyridine-6-carbaldehyde (for a synthesis see WO2004058144, Example 61) (40 mg), sodium acetate (86 mg), acetic acid (12 drops) in methanol (5 mL) and chloroform (5 mL) were stirred with 3 A sieves at room temperature for 3 hours. Sodium cyanoborohydride (54 mg) was added and the mixture stirred overnight, basified and extracted with 10% methanol in dichloromethane. The extracts were dried (sodium sulphate) and evaporated. Chromatography on silica gel (0-20% methanol-DCM), was followed by mass directed autoprep purification (M 487; eluent: acetonitrile-water-formic acid). The product was converted to the hydrochloride to give the title compound (11.2 mg).

¹H NMR δ(CD₃OD) 3.90 (3H, s), 4.10-4.20 (1H, m), 4.25-4.35 (1H, m), 4.40-4.55 (1H, m), 4.57 (2H, s), 6.15 (2H, s), 6.96 (1H, d, J 10 Hz), 7.95 (1H, d, J 10 Hz), 8.01 (1H, s), 8.27 (1H, s), 8.15 (1H, s), remainder of spectrum obscured by solvent.

LC/MS (+ve ion electrospray): m/z 488 (M+H)⁺.

### Example 7

### 6-{[({(3S,4S)-1-[2-(3-Chloro-5-methyl-6-oxo-5,6-dihydro-1,5-naphthyridin-4-yl)ethyl]-4-hydroxy-3-pyrrolidinyl}methyl)amino]methyl}-2H-pyrido[3,2-b][1,4]thiazin-3(4H)-one dihydrochloride

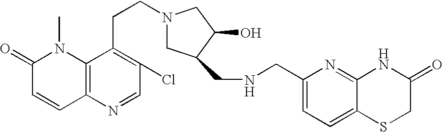

The title compound was prepared from N-({(3S,4S)-1-[2-(3-chloro-5-methyl-6-oxo-5,6-dihydro-1,5-naphthyridin-4-yl)ethyl]-4-hydroxy-3-pyrrolidinyl}methyl)-2,2,2-trifluoroacetamide (50 mg) and 3-oxo-3,4-dihydro-2H-pyrido[3,2-b][1,4]thiazine-6-carboxaldehyde (for a synthesis see WO2003087098, Example 301(d)) (22.5 mg) as the aldehyde component by the general method of Example 3(e). The product, after work-up, was subjected to mass-directed autoprep purification (M 514; eluent: acetonitrile-water-formic acid) followed by treatment with 4M hydrogen chloride in dioxan. The solution was evaporated to give the title compound (11 mg), after trituration with ether.

¹H NMR δ(CD₃OD-CDCl₃) 3.56 (2H, s), 3.60-3.80 (7H, m), 3.86 (3H, s), 4.07 (1H, m), 4.37 (2H, s), 4.64 (1H, m), 3.45 (2H, s), 6.90 (1H, d), 7.14 (1H, d), 7.82 (1H, d), 7.92 (1H, d) and 8.58 (1H, s). (several signals obscured by solvent).

LC/MS (+ve ion electrospray): m/z 515/7 (M+H)⁺

### Example 8

### 7-Chloro-1-methyl-8-[2-((3R)-3-{[([1,3]oxathiolo[5,4-c]pyridin-6-ylmethyl)amino]methyl}-1-pyrrolidinyl)ethyl]-1,5-naphthyridin-2(1H)-one dihydrochloride

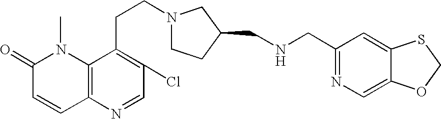

### (a) 1,1-Dimethylethyl(3R)-3-{[(trifluoroacetyl)amino]methyl}-1-pyrrolidinecarboxylate

A solution of 1,1-dimethylethyl(3R)-3-(aminomethyl)-1-pyrrolidinecarboxylate (1.77 g) in DCM (100 mL) with triethylamine (2.60 mL), 4-(dimethylamino)-pyridine (100 mg) and trifluoroacetic anhydride (1.31 mL), was left stirring at room temperature for 2 hours. Water was added and the solution was extracted with DCM, dried (sodium sulphate), evaporated, and chromatographed on silica gel (0-5% methanol-DCM) to afford a foam (2.20 g).

### (b) 2,2,2-Trifluoro-N-[(3S)-3-pyrrolidinylmethyl]acetamide hydrochloride

1,1-Dimethylethyl(3R)-3-{[(trifluoroacetyl)amino]methyl}-1-pyrrolidinecarboxylate (2.20 g) in DCM (30 mL) was treated with 2M hydrogen chloride in dioxane (5.5 mL) and the mixture was stirred for 4 hours and evaporated to give an oil (1.83 g).

### (c) N-{(3S)-1-[2-(3-chloro-5-methyl-6-oxo-5,6-dihydro-1,5-naphthyridin-4-yl)ethyl]-3-pyrrolidinyl}-2,2,2-trifluoroacetamide

Impure (3-chloro-5-methyl-6-oxo-5,6-dihydro-1,5-naphthyridin-4-yl)acetaldehyde (equivalent to ca. 0.084 g of pure material), 2,2,2-trifluoro-N-[(3S)-3-pyrrolidinylmethyl]acetamide hydrochloride (0.160 g), anhydrous sodium acetate (0.33 g) in dry methanol (4 mL), chloroform (4 mL) and acetic acid (10 drops) and 3 A molecular sieves was stirred at room temperature for 2 hours, then sodium cyanoborohydride (0.07 g) was added and the mixture stirred at room temperature for 2 hours. Aqueous sodium bicarbonate was added and the mixture extracted with 10% methanol-chloroform, dried (sodium sulphate), evaporated and chromatographed on silica gel (methanol-DCM) to afford an oil (85 mg).

LC/MS (+ve ion electrospray): m/z 417/9 (M+H)⁺

### (d) Title Compound

Impure N-{(3S)-1-[2-(3-chloro-5-methyl-6-oxo-5,6-dihydro-1,5-naphthyridin-4-yl)ethyl]-3-pyrrolidinyl}-2,2,2-trifluoroacetamide (equivalent to ca. 72 mg pure material) in methanol (2 mL) and water (2 mL) was stirred with anhydrous potassium carbonate (80 mg) for 4 hours at room temperature, then evaporated, and azeotroped with methanol, then toluene. The residue was dissolved in methanol (3 mL), chloroform (3 mL), and acetic acid (7 drops) and treated with anhydrous sodium acetate (120 mg), [1,3]oxathiazolo[5,4-c]pyridine-6-carbaldehyde (for a synthesis see WO2004058144, Example 61) (29 mg) and 3A molecular sieves and stirred at room temperature for 1 hour. Sodium cyanoborohydride (30 mg) was added and the mixture was stirred at room temperature, overnight. Aqueous sodium carbonate was added and the mixture was extracted with 10% methanol-chloroform, dried (sodium sulphate), evaporated and subjected to mass-directed autoprep purification (M 471; eluent: acetonitrile-water-formic acid) followed by treatment with 4M hydrogen chloride in dioxan. The solution was evaporated to give the title compound (53 mg), after trituration with ether.

¹H NMR δ(CD₃OD) 1.0-2.10 (1H,), 2.40-2.60 (1H, m), 3.30-3.65 (ca. 4H, m), 3.75 (1H, m), 3.87 (3H, s), 3.90-4.20 (1H, m), 4.42 (2H, s), 6.00 (2H, s), 6.92 (1H, d), 7.70 (1H, br. s), 7.92 (1H, d), 8.15 (1H, s) and 8.59 (1H, s). (several signals obscured by solvent).

LC/MS (+ve ion electrospray): m/z 472 (M+H)⁺

### Example 9

### 7-Chloro-1-methyl-8-[2-((3S)-3-{[([1,3]oxathiolo[5,4-c]pyridin-6-ylmethyl)amino]methyl}-1-pyrrolidinyl)ethyl]-1,5-naphthyridin-2(1H)-one dihydrochloride

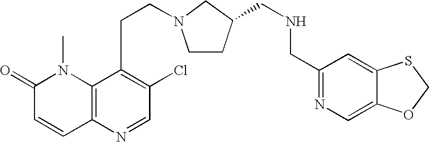

### (a) 1,1-Dimethylethyl(3S)-3-{[(trifluoroacetyl)amino]methyl}-1-pyrrolidinecarboxylate

A solution of 1,1-dimethylethyl(3S)-3-(aminomethyl)-1-pyrrolidinecarboxylate (2.0 g) in DCM (80 mL) with triethylamine (3.0 mL), 4-(dimethylamino)-pyridine (120 mg) and trifluoroacetic anhydride (1.48 mL), was left stirring at room temperature for 4 hours. Water was added and the solution was extracted with DCM, dried (sodium sulphate), evaporated, and chromatographed on silica gel (0-5% methanol-DCM) to afford a foam (3.04 g).

### (b) 2,2,2-Trifluoro-N-[(3R)-3-pyrrolidinylmethyl]acetamide hydrochloride

1,1-Dimethylethyl(3S)-3-{[(trifluoroacetyl)amino]methyl}-1-pyrrolidinecarboxylate (3.0 g) in DCM (30 mL) was treated with 2M hydrogen chloride in dioxane (8 mL) and the mixture was stirred for 4 hours and evaporated to give an oil (2.59 g).

### (c) N-{(3R)-1-[2-(3-chloro-5-methyl-6-oxo-5,6-dihydro-1,5-naphthyridin-4-yl)ethyl]-3-pyrrolidinyl}-2,2,2-trifluoroacetamide

Impure (3-chloro-5-methyl-6-oxo-5,6-dihydro-1,5-naphthyridin-4-yl)acetaldehyde (equivalent to ca. 65 mg of pure material), 2,2,2-trifluoro-N-[(3R)-3-pyrrolidinylmethyl]acetamide hydrochloride (0.120 g), anhydrous sodium acetate (0.2 g) in methanol (3 mL), chloroform (3 mL) and acetic acid (10 drops) and 3 A molecular sieves was stirred at room temperature for 2 hours, then sodium cyanoborohydride (0.06 g) was added and the mixture stirred at room temperature for 2 hours. The mixture was basified and extracted with 10% methanol-chloroform, dried (sodium sulphate), evaporated and chromatographed on silica gel (methanol-DCM) to afford an oil (62 mg).

LC/MS (+ve ion electrospray): m/z 417/9 (M+H)⁺

### (d) Title Compound

N-({(3R)-1-[2-(3-chloro-5-methyl-6-oxo-5,6-dihydro-1,5-naphthyridine-4-yl)ethyl]pyrrolidin-3-yl}-2,2,2-trifluoroacetamide (62 mg), in methanol (3 mL) and water (3 mL) was stirred with anhydrous potassium carbonate (50 mg) overnight at room temperature. The mixture was evaporated and azeotroped with toluene and dried under vacuum. The residue was dissolved in methanol (3 mL), chloroform (3 mL), and acetic acid (10 drops) and treated with sodium acetate (110 mg), [1,3]oxathiazolo[5,4-c]pyridine-6-carbaldehyde (22.5 mg) (for a synthesis see WO2004058144, Example 61) and 3 A molecular sieves and stirred at room temperature for 2 hours. Sodium cyanoborohydride (40 mg) was added and the mixture stirred overnight at room temperature. The mixture was basified and extracted with 10% methanol-dichloromethane, dried (sodium sulfate), evaporated and subjected to mass directed autoprep purification (M 471; eluent: acetonitrile-water-formic acid) followed by treatment with 4M hydrogen chloride in dioxan. The solution was evaporated to give the title compound (37 mg).

LC/MS (+ve ion electrospray): m/z 472 (M+H)⁺.

### Example 10

### 6-[({1-[2-(3-Chloro-5-methyl-6-oxo-5,6-dihydro-1,5-naphthyridin-4-yl)ethyl]-4-piperidinyl}amino)methyl]-2H-pyrido[3,2-b][1,4]oxazin-3(4H)-one dihydrochloride

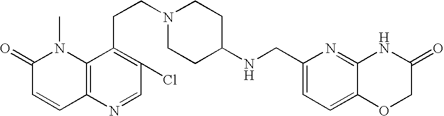

The title compound was prepared from 8-[2(4-amino-1-piperidinyl)ethyl]-7-chloro-1-methyl-1,5-naphthyridin-2(1H)-one free base (obtained by treatment of dihydrochloride with sodium carbonate and extraction with 10% methanol-chloroform, giving impure material, equivalent to ca. 55 mg of pure material) and 3-oxo-3,4-dihydro-2H-pyrido[3,2-b][1,4]oxazine-6-carboxaldehyde (for a synthesis see WO2003087098, Example 31(e)) (30.5 mg) according to the general method of Example 1(j) (total reaction time 5 hr), and was subjected to silica gel chromatography, followed by mass-directed autoprep purification (M 482; eluent: acetonitrile-water-formic acid) followed by treatment with 4M hydrogen chloride in dioxan. The solution was evaporated to give the title compound (47 mg) after trituration with ether.

LC/MS (+ve ion electrospray): m/z 483/5 (M+H)⁺

### Example 11

### 7-Chloro-8-(2-{4-[(2,3-dihydro[1,4]dioxino[2,3-c]pyridin-7-ylmethyl)amino]-1-piperidinyl}ethyl)-1-methyl-1,5-naphthyridin-2(1H)-one dihydrochloride

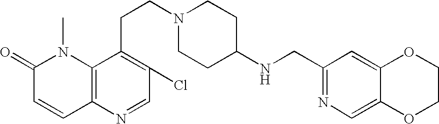

The title compound was prepared from 8-[2-(4-amino-1-piperidinyl)ethyl]-7-chloro-1-methyl-1,5-naphthyridin-2(1H)-one dihydrochloride (40 mg) and 2,3-dihydro[1,4]dioxino[2,3-c]pyridine-7-carboxaldehyde (for a synthesis see WO2004058144, Example 2(c) or WO03/087098, Example 19(d)) (20.6 mg) according to the general method of Example 1(j). The crude product was chromatographed on silica gel (methanol-DCM) to give the free base (38 mg). The free base was reacted with 4M hydrogen chloride in dioxane and the solution was evaporated to give the title compound, after trituration with ether.

LC/MS (+ve ion electrospray): m/z 469/71 (M+H)⁺

### Example 12

### 7-Chloro-8-(2-{4-[(3,4-dihydro-2H-pyrano[2,3-c]pyridin-6-ylmethyl)amino]-1-piperidinyl}ethyl)-1-methyl-1,5-naphthyridin-2(1H)-one dihydrochloride

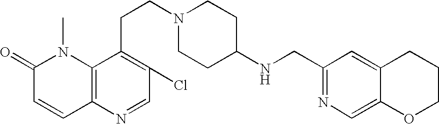

The title compound was prepared from 8-[2-(4-amino-1-piperidinyl)ethyl]-7-chloro-1-methyl-1,5-naphthyridin-2(1H)-one dihydrochloride (40 mg) and 3,4-dihydro-2H-pyrano[2,3-c]pyridine-6-carbaldehyde (for a synthesis see WO2004058144, Example 126(e)) (21 mg) according to the general method of Example 1(j) (total reaction time 7 hr). The crude product was chromatographed on silica gel (methanol-DCM) to give the free base, which was treated with 4M hydrogen chloride in dioxane and the solution was evaporated to give the title compound (18 mg), after trituration with ether.

LC/MS (+ve ion electrospray): m/z 468/470 (M+H)⁺

### Example 13

### 5-[({(3R,4S)-1-[2-(3-Chloro-5-methyl-6-oxo-5,6-dihydro-1,5-naphthyridin-4-yl)ethyl]-3-hydroxy-4-piperidinyl}amino)methyl]-2,3-dihydro-1-benzofuran-7-carbonitrile dihydrochloride

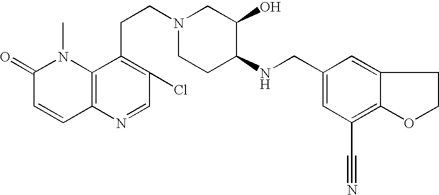

### (a) 7-Bromo-2,3-dihydro-1-benzofuran-5-carbaldehyde

To a solution of 2,3-dihydro-1-benzofuran-5-carbaldehyde (1.0 g, 6.75 mmol) in glacial acetic acid (8 mL) was added sodium acetate (664 mg, 8.1 mmol) and bromine (0.7 ml, 13.5 mmol) slowly at 10° C. The reaction was stirred for 2 hours at ambient temperature. The reaction was diluted with a saturated aqueous solution of sodium thiosulfate (10 mL), washed with a saturated aqueous solution of sodium bicarbonate, and then extracted with ethyl acetate. Organics were combined, dried over sodium sulfate and dried in vacuo to give the desired compound (1.4 g, 91%).

MS (+ve ion electrospray): m/z 227 (M+H)⁺.

### (b) 5-Formyl-2,3-dihydro-1-benzofuran-7-carbonitrile

To a solution of 7-bromo-2,3-dihydro-1-benzofuran-5-carbaldehyde (1.3 g, 4.7 mmol) in dimethylacetamide (2 mL) was added copper(I) cyanide (0.41 g g, 4.7 mmol). The reaction was refluxed for 18 hours, and then concentrated under reduced pressure. The residue was washed well with warm ethyl acetate. The combined ethyl acetate layer were concentrated and dried. The crude product was purified by flash column chromatography (silica gel, 4:1 to 2:1 hexane:ethyl acetate gradient) to afford the desired product (0.5 g, 50%).

MS (+ve ion electrospray): m/z 174 (M+H)⁺.

### (c) Title Compound

This was prepared from 8-{2-[(3R,4S)-4-amino-3-hydroxy-1-piperidinyl]ethyl}-7-chloro-1-methyl-1,5-naphthyridin-2(1H)-one (70 mg) and 5-formyl-2,3-dihydro-1-benzofuran-7-carbonitrile (36 mg) according to the general method of Example 1(j) [except an additional (3 mg) of aldehyde was added at 3 hours and reaction was stirred for a further 1 hour before work-up] and was subjected to mass-directed autoprep purification (M 493; eluent: acetonitrile-water-formic acid) followed by treatment with 4M hydrogen chloride in dioxan. The solution was evaporated to give the title compound (20 mg) after trituration with ether.

LC/MS (+ve ion electrospray): m/z 494/6 (M+H)⁺

### Example 14

### 6-{[({(3R)-1-[2-(3-Chloro-5-methyl-6-oxo-5,6-dihydro-1,5-naphthyridin-4-yl)ethyl]-3-pyrrolidinyl}methyl)amino]methyl}-2H-pyrido[3,2-b][1,4]oxazin-3(4H)-one dihydrochloride

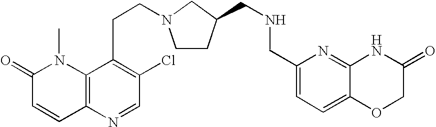

### (a) 8-{2-[(3R)-3-(Aminomethyl)-1-pyrrolidinyl]ethyl}-7-chloro-1-methyl-1,5-naphthyridin-2(1H)-one

N-{(3S)-1-[2-(3-chloro-5-methyl-6-oxo-5,6-dihydro-1,5-naphthyridin-4-yl)ethyl]-3-pyrrolidinyl}-2,2,2-trifluoroacetamide (216 mg) in methanol (8 mL) and water (8 mL) was stirred with potassium carbonate (180 mg) for 3 hours at room temperature. Further potassium carbonate (50 mg) was added and stirring continued overnight. The mixture was evaporated and dried under vacuum.

### (b) Title Compound

8-{2-[(3R)-3-(Aminomethyl)-1-pyrrolidinyl]ethyl}-7-chloro-1-methyl-1,5-naphthyridin-2(1H)-one (ca. 0.3 mmol) was dissolved in methanol (3 mL) and chloroform (3 mL), and acetic acid (5 drops) and treated with sodium acetate (43 mg), 3-oxo-3,4-dihydro-2H-pyrido[3,2-b][1,4]oxazine-6-carboxaldehyde (for a synthesis see WO2003087098 Example 31(e)) (23 mg) and 3 A molecular sieves, then stirred at room temperature 2 hours. Sodium cyanoborohydride (27 mg) was added and the mixture stirred at room temperature overnight. Aqueous sodium carbonate was added and the mixture was extracted with 10% methanol-dichloromethane, dried (sodium sulfate), and evaporated. The crude product was dissolved in methanol-dichloromethane and washed with water (2×), dried (sodium sulfate) and evaporated, then subjected to mass directed autoprep purification (M 482; eluent: acetonitrile-water-formic acid) then converted to the dihydrochloride by treatment of a solution in methanol with 4M HCl/1,4-dioxan and precipitation with ether to give the title compound (16 mg).

LC/MS (+ve ion electrospray): m/z 483 (M+H)⁺.

### Example 15

### 6-{[({(3R)-1-[2-(3-Chloro-5-methyl-6-oxo-5,6-dihydro-1,5-naphthyridin-4-yl)ethyl]-3-pyrrolidinyl}methyl)amino]methyl}-2H-pyrido[3,2-b][1,4]thiazin-3(4H)-one dihydrochloride

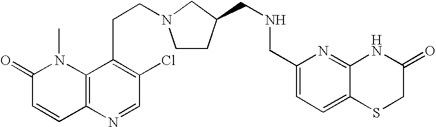

8-{2-[(3R)-3-(Aminomethyl)-1-pyrrolidinyl]ethyl}-7-chloro-1-methyl-1,5-naphthyridin-2(1H)-one (ca. 0.13 mmol) was dissolved in methanol (4 mL) and chloroform (4 mL), and acetic acid (5 drops) and treated with sodium acetate (43 mg), 3-oxo-3,4-dihydro-2H-pyrido[3,2-b][1,4]thiazine-6-carboxaldehyde (for a synthesis see WO2003087098 Example 301(d)) (25 mg) and 3 A molecular sieves then stirred at room temperature for 2 hours. Sodium cyanoborohydride (27 mg) was added and the mixture stirred at room temperature overnight. The mixture was basified and extracted with 10% methanol-dichloromethane, the extracts were washed with water and brine, dried (sodium sulfate), and evaporated, then subjected to mass directed autoprep purification (M 498; eluent: acetonitrile-water-formic acid) then converted to the dihydrochloride by treatment of a solution in methanol with 4M HCl/1,4-dioxan and precipitation with ether to give the title compound (18 mg).

LC/MS (+ve ion electrospray): m/z 499 (M+H)⁺.

### Example 16

### 6-{[({(3R,4R)-1-[2-(3-Chloro-5-methyl-6-oxo-5,6-dihydro-1,5-naphthyridin-4-yl)ethyl]-4-hydroxy-3-pyrrolidinyl}methyl)amino]methyl}-2H-pyrido[3,2-b][1,4]thiazin-3(4H)-one dihydrochloride

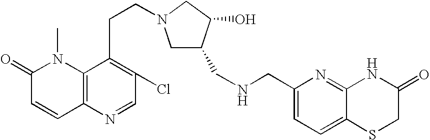

### (a) 1,1-Dimethylethyl(3R,4R)-3-(aminomethyl)-4-hydroxy-1-pyrrolidinecarboxylate

This was prepared from 1,1-dimethylethyl(3R,4R)-3-hydroxy-4-[({[(phenylmethyl)oxy]carbonyl}amino)methyl]-1-pyrrolidinecarboxylate (for a synthesis, see WO 2006/002047 Preparation 24c, enantiomer E2), by the general method of Example 3(a).

LC/MS (+ve ion electrospray): m/z 217 (M+H)⁺

### (b) 1,1-Dimethylethyl(3R,4R)-3-hydroxy-4-{[(trifluoroacetyl)amino]methyl}-1-pyrrolidinecarboxylate

This was prepared from 1,1-dimethylethyl(3R,4R)-3-(aminomethyl)-4-hydroxy-1-pyrrolidinecarboxylate by the general method of Example 3(b).

LC/MS (−ve ion electrospray): m/z 311 (M−H)⁻

### (c) 2,2,2-Trifluoro-N-{[(3S,4R)-4-hydroxy-3-pyrrolidinyl]methyl}acetamide hydrochloride

This was prepared from 1,1-dimethylethyl(3R,4R)-3-hydroxy-4-{[(trifluoroacetyl)amino]methyl}-1-pyrrolidinecarboxylate by the general method of Example 3(c).

LC/MS (+ve ion electrospray): m/z 213 (M+H)⁺

### (d) N-({(3R,4R)-1-[2-(3-chloro-5-methyl-6-oxo-5,6-dihydro-1,5-naphthyridin-4-yl)ethyl]-4-hydroxy-3-pyrrolidinyl}methyl)-2,2,2-trifluoroacetamide

This was prepared from 2,2,2-trifluoro-N-{[(3S,4R)-4-hydroxy-3-pyrrolidinyl]methyl}acetamide and (3-chloro-5-methyl-6-oxo-5,6-dihydro-1,5-naphthyridin-4-yl)acetaldehyde by the general method of Example 3(d).

LC/MS (+ve ion electrospray): m/z 433/5 (M+H)⁺

### (e) Title Compound

This was prepared from the N-({(3R,4R)-1-[2-(3-chloro-5-methyl-6-oxo-5,6-dihydro-1,5-naphthyridin-4-yl)ethyl]-4-hydroxy-3-pyrrolidinyl}methyl)-2,2,2-trifluoroacetamide (70 mg) and 3-oxo-3,4-dihydro-2H-pyrido[3,2-b][1,4]thiazine-6-carboxaldehyde (for a synthesis see WO2003087098, Example 301(d)) (32 mg), as the aldehyde component, by the general method of Example 3(e). The product, after work-up, was subjected to mass-directed autoprep purification (M 515; eluent: acetonitrile-water-formic acid) followed by treatment with 4M hydrogen chloride in dioxane. The solution was evaporated to give the title compound (44 mg), after trituration with ether.

LC/MS (+ve ion electrospray): m/z 515/7 (M+H)⁺

### Example 17

### 7-{[({(3R)-1-[2-(3-Chloro-5-methyl-6-oxo-5,6-dihydro-1,5-naphthyridin-4-yl)ethyl]-3-pyrrolidinyl}methyl)amino]methyl}-2,3-dihydro-1,4-benzodioxin-5-carbonitrile dihydrochloride

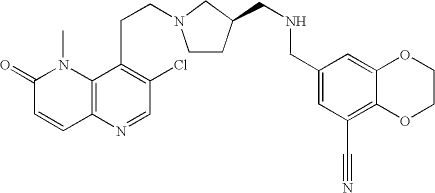

8-{2-[(3R)-3-(Aminomethyl)-1-pyrrolidinyl]ethyl}-7-chloro-1-methyl-1,5-naphthyridin-2(1H)-one (ca. 0.13 mmol) was dissolved in methanol (4 mL) and chloroform (4 mL), and acetic acid (5 drops) and treated with sodium acetate (43 mg), 8-cyano-2,3-dihydro-1,4-benzodioxin-6-carbaldehyde (25 mg) (for a synthesis, see WO 2006/014580, Preparation 13(d) 7-formyl-2,3-dihydro-1,4-benzodioxin-5-carbonitrile) and 3 A molecular sieves then stirred at room temperature 2 hours. Sodium cyanoborohydride (27 mg) was added and the mixture stirred at room temperature overnight. The mixture was basified and extracted with 10% methanol-dichloromethane, the extracts were washed with water and brine, dried (sodium sulfate), and evaporated. The crude product was then subjected to mass directed autoprep purification (M 493; eluent: acetonitrile-water-formic acid) then converted to the dihydrochloride by treatment of a solution in methanol with 4M HCl/1,4-dioxan and precipitation with ether to give the title compound (13 mg).

LC/MS (+ve ion electrospray): m/z 494 (M+H)⁺.

### Example 18

### 7-Chloro-8-[2-((3R)-3-{[(2,3-dihydro[1,4]dioxino[2,3-c]pyridin-7-ylmethyl)amino]methyl}-1-pyrrolidinyl)ethyl]-1-methyl-1,5-naphthyridin-2(1H)-one dihydrochloride

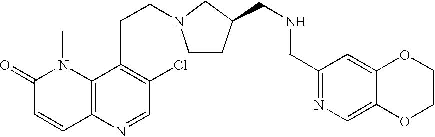

8-{2-[(3R)-3-(Aminomethyl)-1-pyrrolidinyl]ethyl}-7-chloro-1-methyl-1,5-naphthyridin-2(1H)-one (ca. 0.13 mmol) was dissolved in methanol (3 mL) and chloroform (3 mL), and acetic acid (5 drops) and treated with sodium acetate (43 mg), 2,3-dihydro[1,4]dioxino[2,3-c]pyridine-7-carboxaldehyde (for a synthesis see WO2004058144, Example 2(c) or WO03/087098, Example 19(d)) (21 mg), and 3 A molecular sieves then stirred at room temperature 2 hours. Sodium cyanoborohydride (27 mg) was added and the mixture stirred at room temperature overnight. Aqueous sodium carbonate was added and the mixture was extracted with 10% methanol-dichloromethane, dried (sodium sulfate), and evaporated. The crude product was then subjected to mass directed autoprep purification (M 469; eluent: acetonitrile-water-formic acid) then converted to the dihydrochloride by treatment of a solution in methanol with 4M HCl/1,4-dioxane and precipitation with ether to give the title compound (32 mg).

LC/MS (+ve ion electrospray): m/z 470 (M+H)⁺.

### Example 19

### 6-[({1-[2-(5-Methyl-6-oxo-5,6-dihydro-1,5-naphthyridin-4-yl)ethyl]-4-piperidinyl}amino)methyl]-2H-pyrido[3,2-b][1,4]thiazin-3(4H)-one dihydrochloride

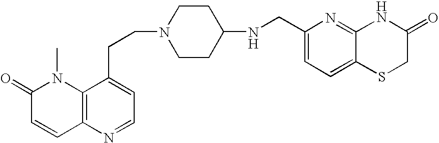

### (a) 2,2-Dimethyl-2,3-dihydro-5H-[1,4,2]oxazasilino[6,5,4-de]-1,5-naphthyridin-5-one

To a suspension of sodium hydride (60% in oil, 9 g, 225 mmol) in dry dimethyl formamide (1000 mL) was added 6-(methyloxy)-1,5-naphthyridin-4-ol(6-methoxy-1H-[1,5]naphthyridin-4-one, for a synthesis see WO2002096907 Example 1) (25 g, 142 mmol) at 0° C. The mixture was stirred for 1 hour, warming to room temperature, then chloro(chloromethyl)dimethylsilane (36 mL) was added dropwise over 10 min. The mixture was stirred 1.5 hours at room temperature, then heated at 100° C. overnight. Solvent was evaporated, then toluene was added and evaporated. Chromatography on silica gel, eluting with 0-30% methanol/dichloromethane, gave the product (34.4 g).

MS (+ve ion electrospray): m/z 233 [M+H]⁺

### (b) 8-Hydroxy-1-methyl-1,5-naphthyridin-2(1H)-one

2,2-Dimethyl-2,3-dihydro-5H-[1,4,2]oxazasilino[6,5,4-de]-1,5-naphthyridin-5-one (34.4 g, 148.4 mmol) was dissolved in 1,4-dioxin (1.65 L) and methanol (850 mL) with warming and sonication, then stirred with caesium fluoride (67.2 g, 444.6 mmol) at reflux temperature for 3 days. Solvent was evaporated and the residue was dissolved in water and methanol. The resulting solution was acidified to pH4-5 with dil. HCl and the solid product was filtered off, washed with a little water, ether and hexane, and dried under vacuum at 50° C. overnight to give the product (17.8 g, 68%).

MS (+ve ion electrospray): m/z 177 [M+H]⁺

### (c) 8-Bromo-1-methyl-1,5-naphthyridin-2(1H)-one

A suspension of 8-hydroxy-1-methyl-1,5-naphthyridin-2(1H)-one (11.86 g, 67.4 mmol) in dimethylformamide (500 mL) was cooled in ice and phosphorus tribromide (10.06 mL, 105.9 mmol) was added dropwise over a few minutes. The resulting mixture was heated at 60° C. overnight, then evaporated. Toluene was added and evaporated off, then the residue was cooled in ice, treated cautiously with aq. sodium bicarbonate until basic, then extracted thoroughly with dichloromethane. The extracts were dried and evaporated to give the product (15.14 g, 94%).

MS (+ve ion electrospray): m/z 239& 241 [M+H]⁺

### (d) 8-Ethenyl-1-methyl-1,5-naphthyridin-2(1H)-one

A solution of 8-bromo-1-methyl-1,5-naphthyridin-2(1H)-one (2.0 g, 8.37 mmol) in 1,2-dimethoxyethane (90 mL) was flushed with argon, then tetrakis(triphenylphosphine)palladium (0) (0.57 g, 0.48 mmol), potassium carbonate (1.41 g), triethenylboroxin pyridine complex (1.84 g, 7.56 mmol) and water (20 mL) were added. The mixture was heated under reflux for 6 hours, then water and diethyl ether were added and the phases were separated. The aqueous phase was extracted several times with ether, then with ethyl acetate, and the combined organics were dried and evaporated. Chromatography on silica gel, eluting with 50-100% ethyl acetate/hexane, gave the product (1.28 g, 82%).

MS (+ve ion electrospray): m/z 187 [M+H]⁺

### (e) 1,1-Dimethylethyl{1-[2-(5-methyl-6-oxo-5,6-dihydro-1,5-naphthyridin-4-yl)ethyl]-4-piperidinyl}carbamate

A mixture of 8-ethenyl-1-methyl-1,5-naphthyridin-2(1H)-one (0.50 g, 2.69 mmol), 1,1-dimethylethyl 4-piperidinylcarbamate (0.58 g, 2.96 mmol) and 1,1,3,3-tetramethylguanidine (0.37 mL) in dimethylformamide (5.5 mL) was heated at 100° C. for 5 days, with more piperidinylcarbamate (0.3 g) added after 48 hours. Solvent was evaporated and the residue was partitioned between dichloromethane and water. The aqueous phase was extracted a few times with dichloromethane, and the extracts were dried and evaporated. Chromatography on silica gel, eluting with 0-10% methanol/dichloromethane, gave the product (0.77 g, 74%).

MS (+ve ion electrospray): m/z 409 [M+Na]⁺, 309 [(M+Na)⁺—C₅H₈O₂], 287 [(M+H)⁺—C₅H₈O₂]

### (f) 8-[2-(4-Amino-1-piperidinyl)ethyl]-1-methyl-1,5-naphthyridin-2(1H)-one

A solution of 1,1-dimethylethyl{1-[2-(5-methyl-6-oxo-5,6-dihydro-1,5-naphthyridin-4-yl)ethyl]-4-piperidinyl}carbamate (0.50 g, 1.29 mmol) in dichloromethane (10 mL) was treated dropwise with trifluoroacetic acid (10 mL). After standing at room temperature for 1.5 hours, the mixture was evaporated. The residue was triturated twice with ether, then dissolved in 10% methanol/dichloromethane (25 ml) and stirred with MP-carbonate resin (Argonaut Technologies, 7.89 mmol) until the mixture was basic to damp pH indicator paper. The resin was filtered off, washed several times alternately with 10% dichloromethane/methanol and methanol, and the liquors evaporated to give the amine (0.54 g, approx. 69% pure).

MS (+ve ion electrospray): m/z 309 [M+Na]⁺

### (g) Title Compound

8-[2-(4-Amino-1-piperidinyl)ethyl]-1-methyl-1,5-naphthyridin-2(1H)-one (approx. 69% pure, 72 mg, 0.17 mmol) and 3-oxo-3,4-dihydro-2H-pyrido[3,2-b][1,4]thiazine-6-carboxaldehyde (for a synthesis, see WO 2003/087098 Ex. 301(d)) (33 mg, 0.17 mmol) were stirred overnight in dry chloroform/methanol (1:1, 4 mL) with acetic acid (6 drops) and 3A molecular sieves. Sodium cyanoborohydride (39 mg) was added and the mixture was stirred at room temperature, with further small portions of aldehyde and cyanoborohydride added, until LC-MS monitoring showed complete consumption of the amine. The mixture was then diluted with dichloromethane, basified with aq. sodium bicarbonate and the phases were separated. The aqueous phase was extracted well with dichloromethane/methanol. The combined organics were dried and evaporated. Chromatography on silica gel, eluting with 0-20% methanol/dichloromethane, gave the free base (53 mg, 67%).

¹H NMR (250 MHz, CDCl₃) δ 8.41 (1H, d), 7.87(1H, d), 7.57 (1H, d), 7.30 (1H, d), 6.98 (1H, d), 6.90 (1H, d), 3.84 (2H, s), 3.83 (3H, s), 3.47 (2H, s), 3.25 (2H, m), 2.93 (2H, m), 2.60 (2H m), 2.52 (1H m), 2.11 (2H, m), 1.92 (2H, m), 1.47 (2H, m),

MS (+ve ion electrospray): m/z 487 [M+Na]⁺

The dihydrochloride salt was prepared by treatment of the free base in chloroform/methanol with two equivalents of 0.4M hydrogen chloride solution in 1,4-dioxane, followed by evaporation of solvent, trituration of the residue with ether and final drying.

### Example 20

### 8-(2-{4-[(2,3-Dihydro[1,4]dioxino[2,3-c]pyridin-7-ylmethyl)amino]-1-piperidinyl}ethyl)-1-methyl-1,5-naphthyridin-2(1H)-one dihydrochloride

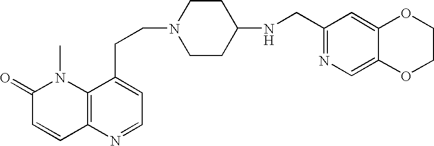

The title compound was prepared from 8-[2-(4-amino-1-piperidinyl)ethyl]-1-methyl-1,5-naphthyridin-2(1H)-one (approx. 69% pure, 72 mg, 0.17 mmol) and 2,3-dihydro[1,4]dioxino[2,3-c]pyridine-7-carboxaldehyde (for a synthesis see WO2004058144, Example 2(c) or WO03/087098, Example 19(d)) according to the general method of Example 19(g).

¹H NMR (free base, 250 MHz, CDCl₃) δ 8.41 (1H, d), 8.09 (1H, s), 7.87 (1H, d), 7.30 (1H, d), 6.89 (1H, d), 6.82 (1H, s), 4.33 (2H, m), 4.27 (2H, m), 3.83 (3H, s), 3.80 (2H, s), 3.24 (2H, m), 2.93 (2H, m), 2.60 (2H m), 2.52 (1H m), 2.11(2H, m), 1.92 (2H, m), 1.47 (2H, m),

MS (+ve ion electrospray): m/z 436 [M+H]⁺

### Example 21

### 1-Methyl-8-(2-{4-[([1,3]oxathiolo[5,4-c]pyridin-6-ylmethyl)amino]-1-piperidinyl}ethyl)-1,5-naphthyridin-2(1H)-one dihydrochloride

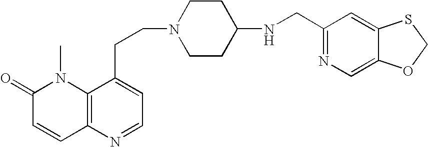

The title compound was prepared from 8-[2-(4-amino-1-piperidinyl)ethyl]-1-methyl-1,5-naphthyridin-2(1H)-one (approx. 69% pure, 72 mg, 0.17 mmol) and [1,3]oxathiazolo[5,4-c]pyridine-6-carbaldehyde (for a synthesis see WO2004058144 Example 61) according to the general method of Example 19(g).

¹H NMR (free base, 250 MHz, CDCl₃) δ 8.41 (1H, d), 8.00 (1H, s), 7.87 (1H, d), 7.30 (1H, d), 7.21 (1H, s), 6.90 (1H, d), 5.73 (2H, s), 3.83 (5H, s), 3.25 (2H, m), 2.93 (2H, m), 2.60 (2H m), 2.53 (1H m), 2.12 (2H, m), 1.92 (2H, m), 1.47 (2H, m),

MS (+ve ion electrospray): m/z 438 [M+H]⁺

### Example 22

### 8-(2-{4-[(2,3-Dihydro[1,4]dioxino[2,3-c]pyridin-7-ylmethyl)amino]-1-piperidinyl}ethyl)-1-methyl-2(1H)-quinolinone hydrochloride

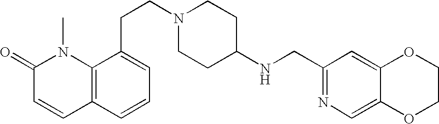

### (a) 1-Methyl-8-(methyloxy)-2(1H)-quinolinone

A suspension of 8-hydroxy-2(1H)-quinolinone (5 g, 31 mmol) in aqueous sodium hydroxide (5 M, 25 mL, 125 mmol) was treated with dimethyl sulphate (5 mL). After 1 hour the solution was treated with a further portion of dimethyl sulphate (17 mL) and heated to reflux overnight. A further portion of dimethyl sulphate (8 mL) was added and the mixture was heated to reflux for 30 minutes. The cooled reaction mixture was partitioned between dichloromethane/chloroform and water. The organic extract was dried and evaporated. The residue was chromatographed eluting with 0-20% methanol in dichloromethane. The impure product (5 g) was subjected to further chromatography eluting with 0-100% ethyl acetate in hexane affording an oil (2.6 g, 44%).

MS (+ve ion electrospray): m/z 190 [M+H]⁺

### (b) 8-Hydroxy-1-methyl-2(1H)-quinolinone

A solution of 1-methyl-8-(methyloxy)-2(1H)-quinolinone (2.6 g, 14 mmol) in 33% hydrogen bromide in acetic acid (20 mL) was heated to reflux for 7 hours. The mixture was cooled to room temperature. Filtration, washing with cold water and drying in vacuo afforded a white solid (2.0 g, 82%).

MS (+ve ion electrospray): m/z 176 [M+H]⁺

### (c) 1-Methyl-2-oxo-1,2-dihydro-8-quinolinyl trifluoromethanesulfonate

A mixture of 8-hydroxy-1-methyl-2(1H)-quinolinone (2.0 g, 11.4 mmol), diisopropylethylamine (2.5 mL, 1.9 g, 14.9 mmol) and N-phenyltrifluoromethanesulphonimide (4.3 g, 12 mmol) in dichloromethane (20 mL) was stirred overnight then evaporated. The residue was chromatographed eluting with 0-100% dichloromethane in hexane then 0-5% methanol in dichloromethane affording a clear oil (2.2 g, 63%).

MS (+ve ion electrospray): m/z 308 [M+H]⁺

### (d) 8-Ethenyl-1-methyl-2(1H)-quinolinone

A solution of 1-methyl-2-oxo-1,2-dihydro-8-quinolinyl trifluoromethanesulfonate (0.98 g, 3.2 mmol), tetrakis(triphenylphosphine)palladium(0) (183 mg, 0.16 mmol), potassium carbonate (438 mg, 3.2 mmol) and triethenylboroxin pyridine complex (1:1) (1.0 g, 4.5 mmol) in dimethoxyethane/water (28 mL/8 mL) was heated under argon at 90° C. for 4 hours. The mixture was diluted with water and extracted twice with ethyl acetate. The combined ethyl acetate extracts were dried and evaporated. The residue was chromatographed eluting with 0-100% ethyl acetate in dichloromethane affording a white solid (434 mg, 74%).

MS (+ve ion electrospray): m/z 186 [M+H]⁺

### (e) 8-[(1R/S)-1,2-Dihydroxyethyl]-1-methyl-2(1H)-quinolinone

A solution of 8-ethenyl-1-methyl-2(1H)-quinolinone (434 mg, 2.3 mmol) in tert-butanol and water (20 mL/20 mL) was treated simultaneously with ADmix alpha (2.5 g) and ADmix beta (2.5 g). After stirring overnight sodium sulphite (5 g) was added and the mixture stirred for 2 hours before being partitioned between ethyl acetate and aqueous sodium bicarbonate solution. The organic extract was dried over magnesium sulphate and evaporated. The residue was warmed, triturated and sonicated with a mixture of dichloromethane, methanol and ethyl acetate. The solvents were decanted off leaving a residue that was dried in vacuo to afford a white solid (305 mg, 59%).

MS (+ve ion electrospray): m/z 220 [M+H]⁺

### (f) (2R/S)-2-Hydroxy-2-(1-methyl-2-oxo-1,2-dihydro-8-quinolinyl)ethyl 4-methylbenzenesulfonate

A suspension of 8-[(1R/S)-1,2-dihydroxyethyl]-1-methyl-2(1H)-quinolinone (300 mg, 1.4 mmol) in dichloromethane/tetrahydrofuran/DMF (10 mL/10 mL/1 mL) was treated with triethylamine (0.3 mL, 2.1 mmol), dibutyl(oxo)stannane (16 mg, 0.07 mmol) and 4-methylbenzenesulfonyl chloride (260 mg, 1.4 mmol). After 4.5 hours the mixture was washed with water and aqueous sodium bicarbonate solution. The organic extract was dried and evaporated to afford a yellow oil (547 mg, 100%).

MS (+ve ion electrospray): m/z 374 [M+H]⁺

### (g) 1-Methyl-8-[(2R/S)-2-oxiranyl]-2(1H)-quinolinone

A solution of (2R/S)-2-hydroxy-2-(1-methyl-2-oxo-1,2-dihydro-8-quinolinyl)ethyl 4-methylbenzenesulfonate (547 mg, 1.4 mmol) in methanol (8 mL) was treated with potassium carbonate (607 mg, 4.4 mmol). After 2 hours the reaction mixture was diluted with water and extracted several times with dichloromethane. The combined organic extracts were dried and evaporated. Chromatography eluting with 0-100% ethyl acetate in dichloromethane afforded a clear oil (174 mg, 59%).

MS (+ve ion electrospray): m/z 202 [M+H]⁺

### (h) (1-Methyl-2-oxo-1,2-dihydro-8-quinolinyl)acetaldehyde

A solution of 1-methyl-8-[(2R/S)-2-oxiranyl]-2(1H)-quinolinone (800 mg, 3.9 mmol) in ethanol (25 mL) was treated with 10% palladium on carbon (500 mg) and hydrogenated for 2 hours giving a complex mixture. Filtration and evaporation gave an oil which was chromatographed on silica eluting with 0-50% methanol in dichloromethane giving the product (73 mg, 9%)

MS (+ve ion electrospray): m/z 202 [M+H]⁺

### (i) 1,1-Dimethylethyl(2,3-dihydro[1,4]dioxino[2,3-c]pyridin-7-ylmethyl){1-[2-(1-methyl-2-oxo-1,2-dihydro-8-quinolinyl)ethyl]-4-piperidinyl}carbamate

A solution of (1-methyl-2-oxo-1,2-dihydro-8-quinolinyl)acetaldehyde (53 mg, 0.26 mmol) and 1,1-dimethylethyl(2,3-dihydro[1,4]dioxino[2,3-c]pyridin-7-ylmethyl)4-piperidinylcarbamate (91 mg, 0.26 mmol) (for a synthesis, see WO2004058144, Example 99(h) in DMF (1 mL) under argon was treated with sodium triacetoxyborohydride (166 mg, 0.78 mmol) and stirred overnight. The mixture was evaporated to dryness and the residue partitioned between ethyl acetate, brine and aqueous sodium bicarbonate solution. The organic extract was dried and evaporated. Chromatography eluting with 0-20% methanol in dichloromethane afforded a yellow solid (94 mg, 66%).

MS (+ve ion electrospray): m/z 535 [M+H]⁺

### (j) Title Compound

A solution of 1,1-dimethylethyl (2,3-dihydro[1,4]dioxino[2,3-c]pyridin-7-ylmethyl){1-[2-(1-methyl-2-oxo-1,2-dihydro-8-quinolinyl)ethyl]-4-piperidinyl}carbamate (94 mg, 0.17 mmol) in dichloromethane/TFA (2.6 mL/2.6 mL) was stirred for 1 hour then evaporated to dryness. The residue was partitioned between ethyl acetate, brine and aqueous sodium bicarbonate solution. The aqueous phasse was further extracted with ethyl acetate. The organic extract was dried and evaporated affording the free base of the title compound as a yellow oil (56 mg, 73%).

¹H NMR (250 MHz, CDCl₃) δ 8.10 (1H, s), 7.65 (1H, d), 7.40 (1H, t), 7.20 (1H, t), 6.82 (1H, s), 6.65 (1H, d), 4.25-4.38 (4H, m), 3.82 (5H, s), 3.25 (2H, m), 3.00 (2H, m), 2.60 (3H, m), 2.15 (2H, m), 1.90 (2H, m), 1.50 (2H, m),

MS (+ve ion electrospray): m/z 435 [M+H]⁺

This material was converted to the title compound (43 mg) by treatment with 1M hydrochloric acid in ether.

### Example 23

### 5-Methyl-4-(2-{4-[([1,3]oxathiolo[5,4-c]pyridin-6-ylmethyl)amino]-1-piperidinyl}ethyl)-6-oxo-5,6-dihydro-1,5-naphthyridine-3-carbonitrile dihydrochloride

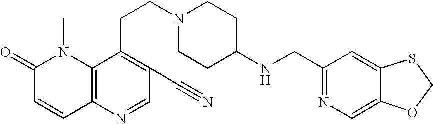

### (a) Ethyl 4-hydroxy-6-methoxy-1,5-naphthyridine-3-carboxylate)

5-Amino-2-methoxypyridine (50 g, 0.40 mol) and diethyl ethoxymethylenemalonate (81.5 ml, 0.40 mol) in ethanol (400 ml) were heated together at 80° C. for 4 hours, then allowed to cool and stirred for 18 hours. The mixture was evaporated to low volume and filtered give a pink solid (88.9 g) A portion of this (43 g) was added to refluxing Dowtherm A (150 ml) and heated at reflux for 12 mins allowing ethanol to boil off the mixture. The mixture was allowed to cool and diluted with pentane. The solid was filtered and washed with DCM to give title compound (25.9 g, 71%).

### (b) Methyl 4-hydroxy-5-methyl-6-oxo-5,6-dihydro-1,5-naphthyridine-3-carboxylate

Ethyl 4-hydroxy-6-methoxy-1,5-naphthyridine-3-carboxylate (34 g, 0.137 mol) was added to ice cooled sodium hydride (8 g, 0.20 mol) in dry DMF (900 ml) and stirred for 1 hour with cooling. Chloro(chloromethyl)dimethylsilane (30 ml. 0.227 mol) was added and stirred at room temperature for 0.5 hours then heated at 100° C. overnight and allowed to cool. The mixture was evaporated and the solid obtained was used without purification. This was heated with cesium fluoride (25 g) in dry methanol (200 ml), dry dioxan (200 ml) and dry THF (200 ml) at 100° C. for 5 days with addition of further cesium fluoride after 2 days. The mixture was evaporated and the residue dissolved in methanol and water and acidified. The precipitate was filtered to give methyl ester (29.8 g).

LC/MS (+ve ion electrospray): m/z 235 (M+H)⁺ Other preparations contained mainly methyl ester with some ethyl ester impurity present.

### (c) Methyl 4-bromo-5-methyl-6-oxo-5,6-dihydro-1,5-naphthyridine-3-carboxylate

Methyl 4-hydroxy-5-methyl-6-oxo-5,6-dihydro-1,5-naphthyridine-3-carboxylate, containing about 15% ethyl 4-hydroxy-5-methyl-6-oxo-5,6-dihydro-1,5-naplithyridine-3-carboxylate (12.8 g, 54.7 mmol) in DMF (200 ml) with phosphorus tribromide (5.99 ml, 63.2 mmol) was stirred for 4 hours then evaporated. The residue was basified with sodium hydrogen carbonate solution and extracted with DCM. Insoluble material was filtered from the mixture. The organic extracts were dried and evaporated to give the product (9.4 g, 58%).

LC/MS (+ve ion electrospray): m/z 297/299 (M+H)⁺, containing ca. 15% ethyl ester.

LC/MS (+ve ion electrospray): m/z 311/313 (M+H)⁺

### (d) Methyl 4-ethenyl-5-methyl-6-oxo-5,6-dihydro-1,5-naphthyridine-3-carboxylate

Methyl 4-bromo-5-methyl-6-oxo-5,6-dihydro-1,5-naphthyridine-3-carboxylate (6.8 g, 22.9 mmol) was stirred in 1,2-dimethoxyethane (200 ml) and flushed with argon, then tetrakis(triphenylphosphine)palladium (0) (1.76 g, 1.9 mmol), potassium carbonate (3.46 g, 22.9 mmol), triethenylboroxin pyridine complex (6 g, 22.5 mmol) and water (50 mL) were added. The mixture was heated at 100° C. for 3 hours. The mixture was diluted with water and extracted with diethyl ether, the extracts were dried, evaporated and chromatographed on silica eluting with 0-4% methanol in DCM to give the product (5.14 g, 92%).

LC/MS (+ve ion electrospray): m/z 245 (M+H)⁺

### (e) 4-Ethenyl-5-methyl-6-oxo-5,6-dihydro-1,5-naphthyridine-3-carboxylic acid

Methyl 4-ethenyl-5-methyl-6-oxo-5,6-dihydro-1,5-naphthyridine-3-carboxylate (5.14 g, 21.1 mmol) in THF (40 ml), water (40 ml) with 2M sodium hydroxide (40 ml) was stirred for 0.5 hours. THF was evaporated and the precipitate was filtered and dried to give the product (3.7 g, 76%).

LC/MS (+ve ion electrospray): m/z 231 (M+H)⁺

### (f) 4-Ethenyl-5-methyl-6-oxo-5,6-dihydro-1,5-naphthyridine-3-carboxamide

4-Ethenyl-5-methyl-6-oxo-5,6-dihydro-1,5-naphthyridine-3-carboxylic acid (3.7 g, 16.1 mmol) was suspended in DCM (75 ml), with DMF (4 drops) and cooled in ice. Oxalyl chloride (2.8 ml, 32.2 mmol) was added and the mixture stirred for 45 mins at room temperature. The mixture was evaporated and the residue in acetone (30 ml) was cooled in ice and ammonia 0.88 (5 ml) was added and the mixture stirred for 3 hours, then evaporated. Water was added to the residue which was stirred for 15 mins then filtered and evaporated. Chromatography on silica eluting with 0-10% methanol in DCM gave the product (4.04 g), containing some impurities.

LC/MS (+ve ion electrospray): m/z 230 (M+H)⁺

### (g) 4-Ethenyl-5-methyl-6-oxo-5,6-dihydro-1,5-naphthyridine-3-carbonitrile

4-Ethenyl-5-methyl-6-oxo-5,6-dihydro-1,5-naphthyridine-3-carboxamide (4.31 g, 18.8 mmol) in DCM (120 ml) with triethylamine (5.0 ml, 35.6 mmol) was cooled in ice and trifluoromethanesulfonic anhydride (7.3 ml, 43.4 mmol) was added. The mixture was stirred and allowed to warm slowly to room temperature. After 2 hours water was added and the mixture extracted with DCM (3×). The extracts were dried, evaporated and chromatographed on silica eluting with 0-10% methanol in DCM to give a brown solid (2.34 g, 59%).

LC/MS (+ve ion electrospray): m/z 212 (M+H)⁺

### (h) 1,1-Dimethylethyl{1-[2-(3-cyano-5-methyl-6-oxo-5,6-dihydro-1,5-naphthyridin-4-yl)ethyl]-4-piperidinyl}carbamate

A mixture of 4-ethenyl-5-methyl-6-oxo-5,6-dihydro-1,5-naphthyridine-3-carbonitrile (2.34 g, 11.1 mmol), 1,1-dimethylethyl 4-piperidinylcarbamate (3.3 g, 16.5 mmol) and 1,1,3,3-tetramethylguanidine (10 drops) in dimethylformamide (20 mL) was heated at 90° C. overnight. The solvent was evaporated and the residue chromatographed on silica gel, eluting with 0-10% methanol in DCM to give the product (3.56 g, 78%).

LC/MS (+ve ion electrospray): m/z 412 (M+H)⁺

### (i) 4-[2-(4-Amino-1-piperidinyl)ethyl]-5-methyl-6-oxo-5,6-dihydro-1,5-naphthyridine-3-carbonitrile hydrochloride

1,1-Dimethylethyl{1-[2-(3-cyano-5-methyl-6-oxo-5,6-dihydro-1,5-naphthyridin-4-yl)ethyl]-4-piperidinyl}carbamate (1.78 g, 4.3 mmol) was dissolved in methanol (25 ml) and chloroform (25 ml) and 4M hydrochloric acid in dioxan (6 ml) was added and stirred for 4 hours. Further 4M hydrochloric acid in dioxan was added (2 ml) and stirring was continued for 3 hours. The mixture was evaporated and stored under high vacuum to give the product, containing impurities (1.32 g, assumed 75% pure).

### (j) Title Compound

4-[2-(4-Amino-1-piperidinyl)ethyl]-5-methyl-6-oxo-5,6-dihydro-1,5-naphthyridine-3-carbonitrile hydrochloride (1.32 g, 3.2 mmol), [1,3]oxathiolo[5,4-c]pyridine-6-carbaldehyde (for a synthesis see WO2004058144, Example 61) (0.53 g, 3.2 mmol), sodium acetate (1.0 g, 12.2 mmol) and sodium triacetoxyborohydride (2.04 g, 9.6 mmol) were combined in DMF (40 ml) and stirred overnight. Further sodium triacetoxyborohydride was added and stirring continued for 4 hours. The mixture was basified with sodium carbonate solution and extracted with 10% methanol in DCM. The extracts were dried, evaporated and chromatographed on silica eluting with 0-10% methanol in DCM to afford the free base.

¹H NMR (CDCl₃) δ 8.66 (s, 1H), 8.00 (s, 1H), 7.88 (d, 1H), 7.10 (s, 1H), 7.01 (d 1H), 5.73 (s, 2H), 3.80 (s, 3H), 3.82 (s, 2H), 3.70-3.45 (m, 2H), 2.95-2.80 (m, 2H), 2.72-2.60 (m, 2H), 2.60-2.45 (m, 1H), 2.26-2.10 (m, 2H), 1.98-1.83 (m, 2H), 1.70-1.30 (m, 2H partly obscured by water).

The free base was dissolved in DCM and treated with 4M HCl in dioxan to give the title compound (0.63 g, 30%) after trituration with diethyl ether.

LC/MS (+ve ion electrospray): m/z 412 (M+H)⁺

### Example 24

### 4-(2-{4-[(2,3-Dihydro[1,4]dioxino[2,3-c]pyridin-7-ylmethyl)amino]-1-piperidinyl}ethyl)-5-methyl-6-oxo-5,6-dihydro-1,5-naphthyridine-3-carbonitrile dihydrochloride

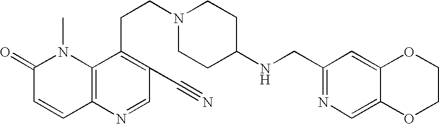

4-[2-(4-Amino-1-piperidinyl)ethyl]-5-methyl-6-oxo-5,6-dihydro-1,5-naphthyridine-3-carbonitrile hydrochloride (0.115 g, 0.37 mmol) was reacted with 2,3-dihydro[1,4]dioxino[2,3-c]pyridine-7-carboxaldehyde (for a synthesis see WO2004058144, Example 2(c) or WO03/087098, Example 19(d)) (0.55 g, 0.33 mmol) in methanol (5 ml) and chloroform (5 ml) with sodium acetate (0.122 g, 1.48 mmol) and acetic acid (16 drops) for 3 hours at room temperature in the presence of 3 A molecular sieves. Sodium cyanoborohydride (0.075 g, 1.19 mmol) was added and the mixture stirred overnight. After being basified with sodium carbonate solution the reaction was extracted with 10% methanol in DCM. The extracts were dried and evaporated. The residue was chromatographed on silica eluting with 5-20% methanol in DCM to give the free base.

¹H NMR (CDCl₃) δ 8.66 (s, 1H), 8.10 (s, 1H), 7.87 (d, 1H), 7.01 (d, 1H), 6.81 (s, 1H), 4.37-4.25 (m, 4H), 3.84 (s, 3H), 3.81 (s, 2H), 3.50 (t, 2H), 2.93-2.84 (m, 2H), 2.58-2.45 (m, 1H), 2.24-2.12 (m, 2H), 1.93-1.84 (m, 2H), 1.49-1.33 (m, 2H).

The free base in DCM was treated with 4M hydrogen chloride in dioxan and the precipitate triturated with ether to give the title compound (0.069 g, 41%).

LC/MS (+ve ion electrospray): m/z 461 (M+H)⁺

### Example 25

### 5-Methyl-6-oxo-4-[2-(4-{[(3-oxo-3,4-dihydro-2H-pyrido[3,2-b][1,4]thiazin-6-yl)methyl]amino}-1-piperidinyl)ethyl]-5,6-dihydro-1,5-naphthyridine-3-carbonitrile dihydrochloride

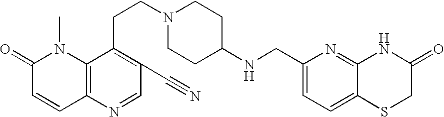

4-[2-(4-Amino-1-piperidinyl)ethyl]-5-methyl-6-oxo-5,6-dihydro-1,5-naphthyridine-3-carbonitrile hydrochloride (0.10 g, 0.26 mmol) was reacted with 3-oxo-3,4-dihydro-2H-pyrido[3,2-b][1,4]thiazine-6-carboxaldehyde (for a synthesis see WO2003087098, Example 301(d)) (0.045 g, 0.23 mmol) with sodium acetate (0.086 g, 1.05 mmol) and acetic acid (11 drops) in methanol (4 ml) and chloroform (4 ml) in the presence of 3 A molecular sieves for 3.5 hours then treated with sodium cyanoborohydride (0.075 g, 1.19 mmol) and stirred overnight. The mixture was basified and extracted with 10% methanol in dichloromethane. Chromatography on silica gel eluting with 0-10% methanol in DCM followed by mass directed autoprep purification (M 489; eluent: acetonitrile-water-formic acid) gave the free base.

¹H NMR (CDCl₃) δ 8.66 (s, 1H), 7.88 (d, 1H), 7.59 (d, 1H), 7.01 (d, 1H), 6.98 (s, 1H), 3.90 (s, 2H), 3.83 (s, 3H), 3.58-3.44 (m, 5H), 2.99-2.88 (m, 2H), 2.74-2.57 (m, 3H), 2.28-2.17 (m, 2H), 2.00-1.90 (m, 2H), 1.60-1.47 (m, 2H).

The free base in DCM was treated with 4M hydrogen chloride in dioxan and the precipitate triturated with ether to give the title compound (0.038 g, 30%).

LC/MS (+ve ion electrospray): m/z 490 (M+H)⁺

### Example 26

### 4-[2-((3R,4S)-3-Hydroxy-4-{[(3-oxo-3,4-dihydro-2H-pyrido[3,2-b][1,4]thiazin-6-yl)methyl]amino}-1-piperidinyl)ethyl]-5-methyl-6-oxo-5,6-dihydro-1,5-naphthyridine-3-carbonitrile dihydrochloride

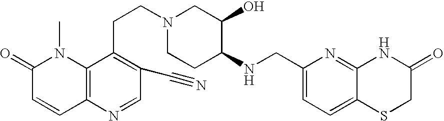

### (a) 4-{2-[(3R,4S)-4-Amino-3-hydroxy-1-piperidinyl]ethyl}-5-methyl-6-oxo-5,6-dihydro-1,5-naphthyridine-3-carbonitrile hydrochloride

4-Ethenyl-5-methyl-6-oxo-5,6-dihydro-1,5-naphthyridine-3-carbonitrile (0.2 g, 0.95 mmol) and 1,1-dimethylethyl[(3R,4S)-3-hydroxy-4-piperidinyl]carbamate (for a synthesis see WO 2004/058144 Example 5(c) cis-(3-hydroxy-piperidin-4-yl)-carbamic acid tert-butyl ester enantiomer 1) (0.32 g, 1.48 mmol) in DMF (2 ml) and 1,1,3,3-tetramethylguanidine (3 drops) were heated at 90° C. overnight then evaporated and chromatographed on silica gel eluting with 0-10% methanol in DCM to give 1,1-dimethylethyl{(3R,4S)-1-[2-(3-cyano-5-methyl-6-oxo-5,6-dihydro-1,5-naphthyridin-4-yl)ethyl]-3-hydroxy-4-piperidinyl}carbamate (0.32 g, 79%).

LC/MS (+ve ion electrospray): m/z 428 (M+H)⁺.

This was dissolved in methanol (10 ml) and chloroform (10 ml) and stirred with 4M hydrogen chloride in dioxan for 3 hours. The mixture was then evaporated to give the product (0.248 g).

LC/MS (+ve ion electrospray): m/z 328 (M+H)⁺

### (b) Title Compound

4-{2-[(3R,4S)-4-Amino-3-hydroxy-1-piperidinyl]ethyl}-5-methyl-6-oxo-5,6-dihydro-1,5-naphthyridine-3-carbonitrile hydrochloride (0.08 g, 0.21mmol) and 3-oxo-3,4-dihydro-2H-pyrido[3,2-b][1,4]thiazine-6-carboxaldehyde (for a synthesis see WO2003087098, Example 301(d)) (0.040 g, 0.21 mmol) in chloroform (1 ml), methanol (1 ml) and acetic acid (10 drops) with sodium acetate (70 mg, 0.82 mmol) and 3 A molecular sieves were stirred for 2 hours. Sodium cyanoborohydride (70 mg, 1.11 mmol) was added and the mixture stirred at room temperature overnight. The mixture was basified and extracted with 10% methanol in chloroform. The extracts were dried and evaporated then chromatographed on silica gel eluting with 0-10% methanol in DCM to give the free base (43 mg).

¹H NMR (CDCl₃) δ 8.68 (s, 1H), 7.90 (d, 1H), 7.59 (d, 1H), 7.02 (d, 1H), 6.96 (d, 1H), 3.86 (s, 2H), 3.83 (s, 3H), 3.60-3.34 (m, 5H), 3.12-2.85 (m, 2H), 2.74-2.20 (m, 7H), 1.85-1.62 (m, 2H).

This was dissolved in DCM and 4M hydrogen chloride in dioxan was added. The precipitate was triturated with ether and dried to give the title compound (41 mg, 39%). LC/MS (+ve ion electrospray): m/z 506 (M+H)⁺

### Example 27

### 4-(2-{(3R,4S)-4-[(2,3-Dihydro[1,4]dioxino[2,3-c]pyridin-7-ylmethyl)amino]-3-hydroxy-1-piperidinyl}ethyl)-5-methyl-6-oxo-5,6-dihydro-1,5-naphthyridine-3-carbonitrile dihydrochloride

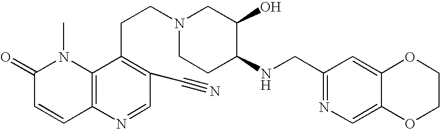

4-{2-[(3R,4S)-4-Amino-3-hydroxy-1-piperidinyl]ethyl}-5-methyl-6-oxo-5,6-dihydro-1,5-naphthyridine-3-carbonitrile hydrochloride (0.08 g, 0.21 mmol) and 2,3-dihydro[1,4]dioxino[2,3-c]pyridine-7-carboxaldehyde (for a synthesis see WO2004058144, Example 2(c) or WO03/087098, Example 19(d)) (0.034 g, 0.21 mmol) in chloroform (1 ml), methanol (1 ml) and acetic acid (10 drops) with sodium acetate (70 mg, 0.82 mmol) and 3A molecular sieves were stirred for 2 hours. Sodium cyanoborohydride (70 mg, 1.11 mmol) was added and the mixture stirred at room temperature overnight. The mixture was basified and extracted with 10% methanol in chloroform. The extracts were dried and evaporated, then chromatographed on silica gel eluting with 0-10% methanol in DCM to give the free base (11 mg).

¹H NMR (CDCl₃) δ 8.68 (s, 1H), 7.90 (d, 1H), 7.59 (d, 1H), 7.02 (d, 1H), 6.96 (d, 1H), 3.86 (s, 2H), 3.83 (s, 3H), 3.60-3.34 (m, 5H), 3.12-2.85 (m, 2H), 2.74-2.20 (m, 7H), 1.85-1.62 (m, 2H).

This was dissolved in DCM and 4M hydrogen chloride in dioxan added. The precipitate was triturated with ether and dried to give the title compound (11 mg, 10%).

LC/MS (+ve ion electrospray): m/z 477 (M+H)⁺

### Example 28

### 4-(2-{(3R,4S)-3-Hydroxy-4-[([1,3]oxathiolo[5,4-c]pyridin-6-ylmethyl)amino]-1-piperidinyl}ethyl)-5-methyl-6-oxo-5,6-dihydro-1,5-naphthyridine-3-carbonitrile dihydrochloride

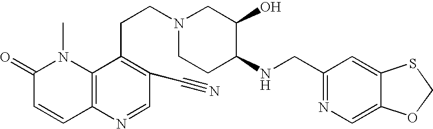

4-{2-[(3R,4S)-4-Amino-3-hydroxy-1-piperidinyl]ethyl}-5-methyl-6-oxo-5,6-dihydro-1,5-naphthyridine-3-carbonitrile hydrochloride (0.08 g, 0.21 mmol) and [1,3]oxathiolo[5,4-c]pyridine-6-carbaldehyde (for a synthesis see WO2004058144, Example 61) (0.034 g, 0.21 mmol) in chloroform (1 ml), methanol (1 ml) and acetic acid (10 drops) with sodium acetate (70 mg, 0.82 mmol) and 3 A sieves were stirred for 2 hours. Sodium cyanoborohydride (70 mg, 1.11 mmol) was added and the mixture stirred at room temperature overnight. The mixture was basified and extracted with 10% methanol in chloroform. The extracts were dried and evaporated, then chromatographed on silica gel eluting with 0-10% methanol in DCM to give the free base (43 mg).

¹H NMR (CDCl₃) δ 8.68 (s, 1H), 7.90 (d, 1H), 7.59 (d, 1H), 7.02 (d, 1H), 6.96 (d, 1H), 5.74 (s, 2H), (3.86 (s, 2H), 3.83 (s, 3H), 3.60-3.34 (m, 5H), 3.12-2.85 (m, 2H), 2.74-2.20 (m, 7H), 1.85-1.62 (m, 2H).

This was dissolved in DCM and 4M hydrogen chloride in dioxan was added. The precipitate was triturated with ether and dried to give the title compound (41 mg, 41%).

LC/MS (+ve ion electrospray): m/z 479 (M+H)⁺

### Example 29

### 4-{2-[(3S,4S)-3-Hydroxy-4-({[(3-oxo-3,4-dihydro-2H-pyrido[3,2-b][1,4]thiazin-6-yl)methyl]amino}methyl)-1-pyrrolidinyl]ethyl}-5-methyl-6-oxo-5,6-dihydro-1,5-naphthyridine-3-carbonitrile dihydrochloride

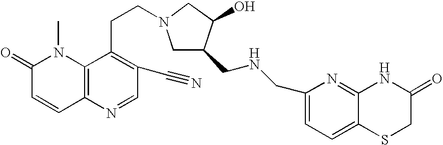

### (a) N-({(3S,4S)-1-[2-(3-Cyano-5-methyl-6-oxo-5,6-dihydro-1,5-naphthyridin-4-yl)ethyl]-4-hydroxy-3-pyrrolidinyl}methyl)-2,2,2-trifluoroacetamide

2,2,2-Trifluoro-N-{[(3R,4S)-4-hydroxy-3-pyrrolidinyl]methyl}acetamide hydrochloride (262 mg, 1.13 mmol) in dichloromethane (5 ml) with triethylamine (0.24 ml) was stirred for 30 minutes then evaporated and dried under vacuum for 1 hour. This was diluted with DMF (5 ml) and 4-ethenyl-5-methyl-6-oxo-5,6-dihydro-1,5-naphthyridine-3-carbonitrile (216 mg, 1.02 mmol) was added and mixture was heated at 90° C. After 30 minutes 1,1,3,3-tetramethylguanidine was added and heating continued overnight. Further 1,1,3,3-tetramethylguanidine (3 drops) was added and the mixture heated for a further 5 hours then evaporated. Chromatography on silica gel the product (172 mg) as a brown oil.

LC/MS (+ve ion electrospray): m/z 424 (M+H)⁺

### (b) 4-{2-[(3S,4S)-3-(Aminomethyl)-4-hydroxy-1-pyrrolidinyl]ethyl}-5-methyl-6-oxo-5,6-dihydro-1,5-naphthyridine-3-carbonitrile

N-({(3S,4S)-1-[2-(3-cyano-5-methyl-6-oxo-5,6-dihydro-1,5-naphthyridin-4-yl)ethyl]-4-hydroxy-3-pyrrolidinyl}methyl)-2,2,2-trifluoroacetamide (219 mg 0.52 mmol) in methanol (5 ml) and water (5 ml) was stirred with potassium carbonate (0.62 g, 4.5 mmol) for 3 hours. The mixture was evaporated, azeotroped with methanol and toluene and stored under vacuum.

### (c) Title Compound

4-{2-[(3S,4S)-3-(Aminomethyl)-4-hydroxy-1-pyrrolidinyl]ethyl}-5-methyl-6-oxo-5,6-dihydro-1,5-naphthyridine-3-carbonitrile (85 mg, 0.26 mmol) and 3-oxo-3,4-dihydro-2H-pyrido[3,2-b][1,4]thiazine-6-carboxaldehyde (for a synthesis, see WO 2003/087098 Ex. 301(d)) (45 mg, 0.23 mmol) in methanol (1 ml), chloroform (1 ml), acetic acid (12 drops), with sodium acetate (90 mg, 1.09 mmol) was stirred for 3 hours. Sodium cyanoborohydride (90 mg, 1.42 mmol) was added and the mixture stirred overnight. After being basified the mixture was extracted with 10% methanol in DCM and the extracts were dried and evaporated. Chromatography on silica gel, eluting with 0-20% methanol in DCM, and then mass directed autoprep purification (M 505; eluent: acetonitrile-water-formic acid), followed by conversion to the dihydrochloride by treatment with 4M HCl/1,4-dioxan and precipitation with ether, gave the title compound (16 mg).

¹H NMR (CDCl₃) δ 8.81 (s, 1H), 7.98 (d, 1H), 7.81 (d, 1H), 7.14 (d, 1H), 7.04 (d, 1H), 4.67 (d, 1H), 4.36 (s, 2H), 4.37(m, 1H), 3.88 (s, 3H), 3.90-3.45 (m, 6H), 3.45-2.80 (m, 7H).

LC/MS (+ve ion electrospray): m/z 506 (M+H)⁺

### Example 30

### 4-[2-((3S,4S)-3-Hydroxy-4-{[([1,3]oxathiolo[5,4-c]pyridin-6-ylmethyl)amino]methyl}-1-pyrrolidinyl)ethyl]-5-methyl-6-oxo-5,6-dihydro-1,5-naphthyridine-3-carbonitrile dihydrochloride

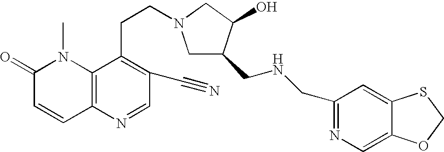

4-{2-[3S,4S)-3-(Aminomethyl)-4-hydroxy-1-pyrrolidinyl]ethyl}-5-methyl-6-oxo-5,6-dihydro-1,5-naphthyridine-3-carbonitrile (85 mg, 0.26 mmol) and [1,3]oxathiolo[5,4-c]pyridine-6-carbaldehyde (for a synthesis see WO2004058144, Example 61) (0.38 g, 0.22 mmol) in methanol (1 ml), chloroform (1 ml), acetic acid (12 drops), with sodium acetate (90 mg, 1.09 mmol) was stirred for 3 hours. Sodium cyanoborohydride (90 mg, 1.42 mmol) was added and the mixture stirred overnight. After being basified the mixture was extracted with 10% methanol in DCM and the extracts were dried and evaporated. Chromatography on silica eluting with 0-20% methanol in DCM gave the free base.

¹H NMR (CDCl₃) δ 8.66 (s, 1H), 8.01 (s, 1H), 7.87 (d, 1H), 7.11 (s, 1H), 7.00 (d, 1H), 5.76 (s, 2H), 4.46 (m, 1H), 3.95-2.30 (m, 17H).

The free base was converted to the title compound (30 mg) by treatment with 4M HCl/1,4-dioxan and precipitation with ether.

LC/MS (+ve ion electrospray): m/z 479 (M+H)⁺

### Example 31

### 8-(2-{4-[(6,7-Dihydro[1,4]dioxino[2,3-c]pyridazin-3-ylmethyl)amino]-1-piperidinyl}ethyl)-7-fluoro-1-methyl-2(1H)-quinolinone fumarate

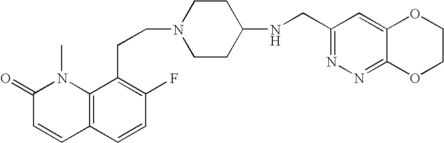

### (a) 3,4,6-Trichloropyridazine

This was prepared by a slight variation on the method of Kasnar et al, Nucleosides & Nucleotides, (1994), 13(1-3), 459-79. Hydrazine sulphate salt (51 g) was suspended in water (250 ml), heated to reflux and bromomaleic anhydride (90.38 g) was added dropwise. The mixture was heated at reflux for 4 h then cooled to room temperature. The reaction was repeated with 29 g hydrazine sulphate, 53 g bromomaleic anhydride and 130 ml water. The precipitates were collected by filtration, washed with water and acetone and dried as a combined batch in vacuo to afford 4-bromo-1,2-dihydro-3,6-pyridazinedione as a white solid (113 g).

The solid, divided into two batches, was treated with phosphorus oxychloride (2×200 ml) and heated to reflux for 3.5 h. The mixture was cooled, evaporated and azeotroped with toluene. The residue was partitioned between dichloromethane and saturated aqueous sodium bicarbonate solution and extracted with DCM twice more. The organic extracts were dried and evaporated. This residue was re-dissolved in dichloromethane, and chromatographed on silica gel (300 g) (DCM as eluent) to give a white solid (101.5 g, 87%).

(LC/MS analysis showed ca. 20-30% impurity, isomers of bromo-dichloropyridazine).

MS (+ve ion electrospray) m/z 184/185/186 (MH+), trichloropyridazine

MS (+ve ion electrospray) m/z 228/229/231 (MH+), bromo-dichloropyridazine.

### (b) 2-[(3,6-Dichloro-4-pyridazinyl)oxy]ethanol

A solution of ethylene glycol (55 ml) in tetrahydrofuran (200 ml) was treated at around 0° C. (ice bath cooling) with sodium hydride (60% dispersion in oil, 5.9 g) over 40 minutes. After the addition was complete, 3,4,6-trichloropyridazine (27 g) containing isomers of bromo-dichloropyridazine as impurity was added portionwise and washed in with more dry THF (50 ml) and the mixture was stirred at 0° C. for 1 hour and then at room temperature overnight. The mixture was concentrated (to ⅓ volume) then diluted with aqueous sodium bicarbonate solution and extracted with chloroform (5×) and ethyl acetate (3×). The combined organic extracts were washed with water, dried over sodium sulphate and evaporated and the solids filtered off and washed with CHCl3 (×3) and dried in a vacuum oven overnight at 40° C. affording a white solid (25.5 g, 83%), containing some bromo-derivative (10-15%).

MS (+ve ion electrospray) m/z 209/211 (MH+).

MS (+ve ion electrospray) m/z 255/7 (MH+), bromo-derivative.

### (c) 3-Chloro-6,7-dihydro[1,4]dioxino[2,3-c]pyridazine

A solution of 2-[(3,6-dichloro-4-pyridazinyl)oxy]ethanol containing some bromo-derivative (15.46 g; 0.0703 mol) in dry 1,4-dioxane (1.2 L) was treated with lithium hydride (2.3 g; 0.28 mol) in portions and stirred at room temperature for 1 hour under argon, then heated at 110° C. overnight. The reaction mixture was quenched with wet 1,4-dioxane, then iced-water. The solution was evaporated to half volume, taken to pH 8 with 5M hydrochloric acid and evaporated to dryness. Water was added and the residue was extracted 5× with chloroform, dried (sodium sulphate) and evaporated to afford a white solid (12.4 g, ca. 77%) (containing ca. 15% of a bromo species).

MS (+ve ion electrospray) m/z 173/5 (Cl MH+); 217/9 (Br MH+)

### (d) 3-Ethenyl-6,7-dihydro[1,4]dioxino[2,3-c]pyridazine

A solution of 3-chloro-6,7-dihydro[1,4]dioxino[2,3-c]pyridazine (13.6 g, 0.079 mol) containing ca. 15% of a bromo species in dimethoxyethane (400 ml) was degassed under argon for 10 min then tetrakis(triphenylphosphine)palladium (0) (2 g), potassium carbonate (10.33 g), triethenylboroxin pyridine complex (11.32 g) and water (55 ml) were added. The mixture was heated at 95° C. for 48 h and cooled and evaporated to dryness. The mixture was treated with aqueous sodium bicarbonate solution and extracted (5×) with DCM. Extracts were dried (sodium sulphate), evaporated and the residue chromatographed on silica gel (500 g), eluting with 0-100% ethyl acetate/hexane, affording the product (6.43 g, 50%); [also some impure fractions (1.8 g)]

MS (+ve ion electrospray) m/z 165 (MH+).

### (e) 6,7-Dihydro[1,4]dioxino[2,3-c]pyridazine-3-carbaldehyde

A solution of 3-ethenyl-6,7-dihydro[1,4]dioxino[2,3-c]pyridazine (11.58 g) in 1,4-dioxane/water (600 ml/180 ml), cooled in ice, was treated with an aqueous solution of osmium tetroxide (4% w/v, 25 ml) and sodium periodate (43 g). This mixture was allowed to warm to room temperature and after 7 h under stirring the mixture was evaporated to dryness and azeotroped with 1,4-dioxane. Silica gel, 1,4-dioxane and chloroform were added and the mixture was evaporated to dryness overnight, then added to a silica column (400 g) and chromatographed, eluting with chloroform then 0-100% ethyl acetate in hexane, to afford a white solid (7.55 g, 64%).

MS (+ve ion electrospray) m/z 167 (MH+).

### (f) N-(2-Bromo-3-fluorophenyl)-3-phenyl-2-propenamide

To a mixture of 2-bromo-3-fluoroaniline (99.9 g, 0.525 mmol) and potassium carbonate (105.2 g, 0.76 mmol) in 1:1 acetone/water (500 mL) at 0-5° C. was added cinnamoyl chloride (88 g) A further 200 mL of acetone was added and the mixture was stirred for 1 h. Water (500 mL) was added and the mixture was extracted with ethyl acetate (3×1000 mL). The organic extracts were washed with water and brine, dried and evaporated to give the amide (163.4 g, 97%).

MS (+ve ion electrospray) m/z 320,322 (MH+).

### (g) 8-Bromo-7-fluoro-2(1H)-quinolinone

A mixture of N-(2-bromo-3-fluorophenyl)-3-phenyl-2-propenamide (86.9 g, 0.221 moles) and aluminium chloride (75 g, 0.563 moles) in chlorobenzene (410 mL) was heated at 85° C. for 2 h, then stood at room temperature for 18 h. After combining with a similar mixture prepared from 76.5 g of the propenamide and 66 g aluminium chloride, the resultant solution was poured onto ice/water (approx. 2.5 L) with overhead stirring, then extracted with 5% methanol/ethyl acetate (2×1 L). The extracts were dried and evaporated. The residue was triturated with ether and the product was filtered off and dried. Re-extraction of the aqueous phase with 10% methanol/dichloromethane (approx. 1.2 L), evaporation of the extracts and trituration as above gave a further crop of product, total yield 82.4 g (67%).

MS (+ve ion electrospray) m/z 241,243 (MH+).

### (h) 8-Bromo-7-fluoro-1-methyl-2(1H)-quinolinone

8-Bromo-7-fluoro-2(1H)-quinolinone (82.43 g, 0.34 moles) in dimethylformamide (1 L) was treated with potassium carbonate (94 g, 0.68 moles) and iodomethane (25.5 mL 0.4 moles) and the mixture was stirred at room temperature for 3 days. The mixture was evaporated and the residue was partitioned between ethyl acetate and water. The organic phase was washed with water and the crude product was chromatographed on silica, eluting with 25% ethyl acetate/petrol (Bp 40-60° C.) to give the major product (8-bromo-7-fluoro-2-(methyloxy)quinoline, 77.1 g), then eluting again with ethyl acetate to give the 1-methylquinolinone (8.2 g, 9%).

MS (+ve ion electrospray) m/z 255,257 (MH+).

### (i) 7-Fluoro-1-methyl-8-(2-propen-1-yl)-2(1H)-quinolinone

A mixture of 8-bromo-7-fluoro-1-methyl-2(1H)-quinolinone (4 g, 15.6 mmol), allyltri-n-butyltin (5.1 mL, 16.5 mmol) and caesium fluoride (5.1 g, 33.6 mmol) in 1,4-dioxane 50 mL) was stirred while a stream of argon was passed through for 15 min. Tris(dibenzylideneacetone)dipalladium(0) (0.16 g) and bis(tri-tert-butylphosphine)palladium(0) (0.16 g) were added, the mixture was degassed again as above and then heated at approx. 75° C. for 3 h (briefly reaching reflux temperature in the first 20 min of heating). The mixture was diluted with ethyl acetate/brine, the aqueous phase was extracted with ethyl acetate and the organic fractions were dried and evaporated. Chromatography on silica, eluting with 0-55% methanol/dichloromethane gave a brown oil (4.9 g, contained some butyl impurities).

MS (+ve ion electrospray) m/z 218 (MH+).

### (j) (7-Fluoro-1-methyl-2-oxo-1,2-dihydro-8-quinolinyl)acetaldehyde

A mixture of 7-fluoro-1-methyl-8-(2-propen-1-yl)-2(1H)-quinolinone (0.43 g, 2.0 mol), sodium periodate (1.2 g) and osmium tetroxide (4% in water, 0.4 mL) in 1,4-dioxane (20 mL) and water (3.5 mL) was stirred at 0° C.—room temperature for 7 h, then allowed to stand overnight. The mixture was evaporated and the residue was diluted with 1,4-dioxane, evaporated onto silica and chromatographed on silica, eluting with 0-10% methanol/dichloromethane to give the aldehyde (0.31 g, 42%, approx. 60% pure).

MS (+ve ion electrospray) m/z 220 (MH+).

### (k) 1,1-Dimethylethyl{1-[2-(7-fluoro-1-methyl-2-oxo-1,2-dihydro-8-quinolinyl)ethyl]-4-piperidinyl}carbamate

A mixture of (7-fluoro-1-methyl-2-oxo-1,2-dihydro-8-quinolinyl)acetaldehyde (0.31 g, approx. 60% pure, 0.85 mol), and 1,1-dimethylethyl 4-piperidinylcarbamate (0.17 g, 0.85 mmol) in 1:1 dichloromethane/methanol (8 mL) was stirred with 3 A molecular sieves for 5 h. Sodium triacetoxyborohydride (0.54 g) was added and the mixture was stirred for approx. 24 h. The mixture was basified with sodium carbonate and extracted with 10% methanol/dichloromethane. The extracts were dried and evaporated. Chromatography on silica, eluting with 0-10% methanol/dichloromethane gave the product (0.20 g, 58%).

MS (+ve ion electrospray) m/z 404 (MH+).

### (l) 8-[2-(4-Amino-1-piperidinyl)ethyl]-7-fluoro-1-methyl-2(1H)-quinolinone hydrochloride

1,1-Dimethylethyl{1-[2-(7-fluoro-1-methyl-2-oxo-1,2-dihydro-8-quinolinyl)ethyl]-4-piperidinyl}carbamate (0.20 g, 0.5 mmol) in dichloromethane (5 mL) was treated with hydrogen chloride (4M in dioxane, 2 mL). After stirring for 1 h, a further 3 mL of hydrogen chloride solution was added and stirring was continued for 20 min. The mixture was evaporated to give the hydrochloride salt (0.20 g).

MS (+ve ion electrospray) m/z 304 (MH+).

### (m) Title Compound

A mixture of 8-[2-(4-amino-1-piperidinyl)ethyl]-7-fluoro-1-methyl-2(1H)-quinolinone hydrochloride (70 mg, 0.20 mmol), 6,7-dihydro[1,4]dioxino[2,3-c]pyridazine-3-carbaldehyde (30 mg, 0.18 mmol), sodium acetate (0.10 g) and acetic acid (6 drops) in 1:1 dichloromethane/methanol (8 mL) was stirred with 3 A molecular sieves for 5 h. (Polystyrylmethyl)trimethylammonium cyanoborohydride (4 mmol/g, 0.2 g) was added and the mixture was stirred overnight. The mixture was filtered, diluted with sodium carbonate solution and extracted with 10% methanol/dichloromethane three times. The organic fractions were dried and evaporated. Chromatography on silica, eluting with 0-20% methanol/dichloromethane, gave the free base of the title compound (26 mg, 30%).

¹H NMR (250 MHz, CDCl₃) δ7.59 (1H, d), 7.37 (1H, dd), 7.05 (1H, s), 6.97 (1H, dd), 6.62 (1H, d), 4.52 (2H, m), 4.37 (2H, m), 4.01 (2H, s), 3.84 (3H, s), 3.22 (2H, m), 2.96 (2H, m), 2.63 (2H, m), 2.53 (1H, m), 2.13 (2H, m), 1.93 (2H, m), 1.47 (2H, m).

MS (+ve ion electrospray) m/z 454 (MH+).

The free base of the title compound in dichloromethane/methanol was treated with 0.5M fumaric acid in methanol (0.11 mL) and ether was added to give a precipitate. This was collected by centrifugation and dried to give the title fumarate salt (19 mg).

### Example 32

### 6-[({1-[2-(7-Fluoro-1-methyl-2-oxo-1,2-dihydro-8-quinolinyl)ethyl]-4-piperidinyl}amino)methyl]-2H-pyrido[3,2-b][1,4]oxazin-3(4H)-one fumarate

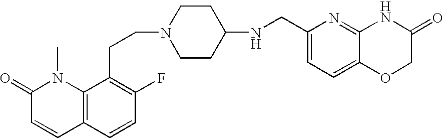

A mixture of 8-[2-(4-amino-1-piperidinyl)ethyl]-7-fluoro-1-methyl-2(1H)-quinolinone hydrochloride (70 mg, 0.20 mmol), 3-oxo-3,4-dihydro-2H-pyrido[3,2-b][1,4]oxazine-6-carboxaldehyde (for a synthesis see WO2003087098 Example 31(e)) (32 mg, 0.18 mmol), sodium acetate (0.10 g) and acetic acid (6 drops) in 1:1 dichloromethane/methanol (8 mL) was stirred with 3A molecular sieves for 5 h. (Polystyrylmethyl)trimethylammonium cyanoborohydride (4 mmol/g, 0.2 g) was added and the mixture was stirred overnight. The mixture was filtered, diluted with sodium carbonate solution and extracted with 10% methanol/dichloromethane three times. The organic fractions were dried and evaporated. Chromatography on silica, eluting with 0-40% methanol/dichloromethane, gave the free base of the title compound (56 mg, 62%).

¹H NMR (250 MHz, CDCl₃) δ7.58 (1H, d), 7.38 (1H, dd), 7.20 (1H, d), 7.03-6.90 (2H, m), 6.62 (1H, d), 4.64 (2H, s), 3.84 (3H, s), 3.81 (2H, s), 3.23 (2H, m), 2.97 (2H, m), 2.61 (2H, m), 2.51 (1H, m), 2.14 (2H, m), 1.92 (2H, m), 1.5 (2H, m part. obscured by water),

MS (+ve ion electrospray) m/z 466 (MH+).

The free base of the title compound in dichloromethane/methanol was treated with 0.5M fumaric acid in methanol (0.24 mL) and ether was added to give a precipitate. This was collected by centrifugation and dried to give the title fumarate salt (51 mg).

### Example 33

### 8-(2-{4-[(2,3-Dihydro[1,4]dioxino[2,3-c]pyridin-7-ylmethyl)amino]-1-piperidinyl}ethyl)-7-fluoro-1-methyl-2(1H)-quinolinone dihydrochloride

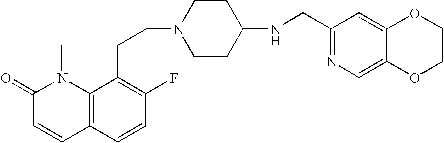

A mixture of 8-[2-(4-amino-1-piperidinyl)ethyl]-7-fluoro-1-methyl-2(1H)-quinolinone hydrochloride (36 mg), 2,3-dihydro[1,4]dioxino[2,3-c]pyridine-7-carboxaldehyde (for a synthesis see WO2004058144, Example 2(c) or WO03/087098 Example 19(d)) (15 mg), sodium acetate (0.05 g) and acetic acid (3 drops) in 1:1 dichloromethane/methanol (8 mL) was stirred with 3A molecular sieves for 5 h. (Polystyrylmethyl)trimethylammonium cyanoborohydride was added and the mixture was stirred over a weekend. The mixture was filtered, basified with sodium carbonate solution and extracted with 10% methanol/dichloromethane three times. The organic fractions were dried and evaporated. Chromatography on silica, eluting with 0-20% methanol/dichloromethane, gave the free base of the title compound (20 mg).

¹H NMR (250 MHz, CDCl₃) δ8.08 (1H, s), 7.61 (1H, d), 7.40 (1H, dd), 6.99 (1H, dd), 6.84 (1H, s), 6.63 (1H, d), 4.34 (2H, m), 4.28 (2H, m), 3.84 (3H, s), 3.79 (2H, s), 3.23 (2H, m), 3.01 (2H, m), 2.65 (2H, m), 2.55 (1H, m), 2.16 (2H, m), 1.95 (2H, m), 1.53 (2H, m).

MS (+ve ion electrospray) m/z 453 (MH+).

The free base of the title compound in dichloromethane/methanol was treated with 4M hydrochloric acid in 1,4-dioxane) and ether was added to give a precipitate. This was collected by centrifugation and dried to give the title dihydrochloride salt (23 mg).

### Example 34

### 7-Fluoro-1-methyl-8-(2-{4-[([1,3]oxathiolo[5,4-c]pyridin-6-ylmethyl)amino]-1-piperidinyl}ethyl)-2(1H)-quinolinone fumarate

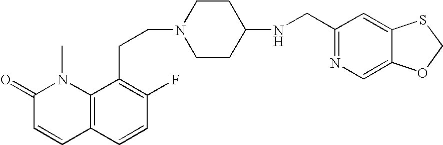

A mixture of 8-[2-(4-amino-1-piperidinyl)ethyl]-7-fluoro-1-methyl-2(1H)-quinolinone hydrochloride (assumed 60% pure, 80 mg, 0.15 mmol), [1,3]oxathiazolo[5,4-c]pyridine-6-carbaldehyde (for a synthesis see WO2004058144, Example 61) (22 mg), sodium acetate (0.08 g) and acetic acid (4 drops) in 1:1 dichloromethane/methanol (6 mL) was stirred with 3 A molecular sieves for 4 h. (Polystyrylmethyl)trimethylammonium cyanoborohydride (0.2 g) was added and the mixture was stirred over a weekend. The mixture was filtered, basified with sodium carbonate solution and extracted with 10% methanol/dichloromethane. The organic extracts were dried and evaporated. Chromatography on silica, eluting with 0-30% methanol/dichloromethane, gave the free base of the title compound (29 mg).

¹H NMR (250MHz, CDCl₃) δ8.01 (1H, s), 7.59 (1H, d), 7.37 (1H, dd), 7.22 (1H, s), 6.97 (1H, dd), 6.62 (1H, d), 5.74 (2H, s), 3.84 (5H, 2x s), 3.24 (2H, m), 2.98 (2H, m), 2.64 (2H, m), 2.55 (1H, m), 2.16 (2H, m), 2.0-1.8 (4H, m, part. obscured by water), 1.48 (2H, m).

MS (+ve ion electrospray) m/z 455 (MH+).

The free base of the title compound in dichloromethane/methanol was treated with 0.5M fumaric acid (0.12 mL) and ether was added to give a small amount of precipitate (3 mg). The liquor was evaporated and the residue was dissolved in water and freeze-dried to give the title fumarate salt (8 mg).

### Example 35

### 3-{[{1-[2-(7-Fluoro-1-methyl-2-oxo-1,2-dihydro-8-quinolinyl)ethyl]-4-piperidinyl}(methyl)amino]methyl}-5H-pyridazino[3,4-b][1,4]thiazin-6(7H)-one fumarate

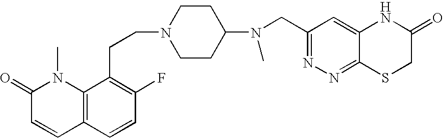

A mixture of 8-[2-(4-amino-1-piperidinyl)ethyl]-7-fluoro-1-methyl-2(1H)-quinolinone hydrochloride (assumed 60% pure, 58 mg, 0.10 mmol), 6-oxo-6,7-dihydro-5H-pyridazino[3,4-b][1,4]thiazine-3-carbaldehyde (purity uncertain, 120 mg) (for a synthesis see WO2004058144, Example 58) (120 mg), sodium acetate (0.10 g) and acetic acid (3 drops) in 1:1 dichloromethane/methanol (8 mL) was stirred with 3A molecular sieves overnight. (Polystyrylmethyl)trimethylammonium cyanoborohydride (0.1 g) was added and the mixture was stirred over a weekend. The mixture was filtered, basified with sodium carbonate solution and extracted with 10% methanol/dichloromethane. The organic extracts were dried and evaporated. Chromatography on silica, eluting with 0-30% methanol/dichloromethane, gave the free base of the title compound (4 mg).

¹H NMR (250 MHz, CDCl₃) δ7.61 (1H, d), 7.41(1H, dd), 7.09 (1H, s), 6.99 (1H, dd), 6.64 (1H, d), 3.87 (2H, s), 3.84 (3H, s), 3.63 (2H, s), 3.25 (2H, m), 3.06 (2H, m), 2.64 (2H, m), 2.42 (1H, m), 2.28 (3H, s), 2.11 (2H, m), 1.66 (2H, m, part. obscured by water).

MS (+ve ion electrospray) m/z 497 (MH+).

The free base of the title compound in methanol was treated with 0.5M fumaric acid in methanol (0.016 mL). The mixture was evaporated and the residue was dissolved in water and freeze-dried to give the title fumarate salt (3 mg).

### Example 36

### 6-{[(1-{2-[1-Methyl-7-(methyloxy)-2-oxo-1,2-dihydro-8-quinolinyl]ethyl}-4-piperidinyl)amino]methyl}-2H-pyrido[3,2-b][1,4]thiazin-3(4H)-one

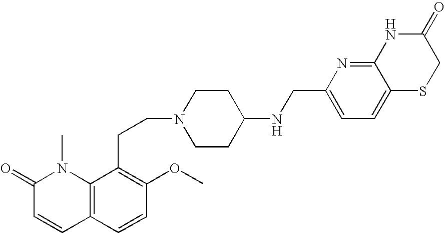

### (a) 8-bromo-1-methyl-7-(methyloxy)-2(1H)-quinolinone

To a solution of 8-bromo-7-fluoro-1-methyl-2(1H)-quinolinone (1.12 g, 4.37 mmol) in methanol (20 mL) was added sodium methoxide (2.36 g, 43.7 mmol). The resulting mixture was stirred at 45° C. overnight. Solvent was removed in vacuo. The crude residue was purified by chromatography on silica gel using a 0-50% ethyl acetate/hexanes gradient to provide the product as white solid (0.6 g; 51%).

MS (+ve ion electrospray) m/z 269 (MH⁺).

### (b) 1-methyl-7-(methyloxy)-8-(2-propen-1-yl)-2(1H)-quinolinone

To a solution of 8-bromo-1-methyl-7-(methyloxy)-2(1H)-quinolinone (0.6 g, 2.24 mmol) in 1,4-dioxane (10 mL) was added allyltributyltin (0.79 g, 2.37 mmol) and caesium fluoride (0.73 g, 4.81 mmol). The resulting mixture was degassed and tris(dibenzylidineacetone)dipalladium(0) (23 mg, 1 mole %) and bis(tri-t-butylphosphine)palladium(0) (23 mg, 2% mol) were added. The resulting mixture was heated at 70° C. overnight. The resulting suspension was filtered through a pad of kieselguhr. The mixture was evaporated under reduced pressure and the residue was extracted with ethyl acetate (3×100 mL). The organic extracts were combined, dried over anhydrous magnesium sulphate, filtered and evaporated under reduced pressure to give a solid which was purified by column chromatography on silica with a 0-10% (1% ammonium hydroxide/methanol) in dichloromethane gradient to give the product as a light-brown oil (0.46 g, 90%).

MS (+ve ion electrospray) m/z 230 (MH⁺).

### (c) [1-methyl-7-(methyloxy)-2-oxo-1,2-dihydro-8-quinolinyl]acetaldehyde

To a solution of 1-methyl-7-(methyloxy)-8-(2-propen-1-yl)-2(1H)-quinolinone (0.46 g, 2 mmol) in a mixture of 1,4-dioxane (25 mL) and water (5 mL) was added sodium periodate (1.28 g, 6 mmol) and osmium tetroxide (0.42 g, 3 mole% as 4% aqueous solution, 10.5 mL). The resulting mixture was stirred at room temperature overnight. The mixture was evaporated under reduced pressure.

The solid product (0.15 g, 32%) was used without further purification.

MS (+ve ion electrospray) 232 (MH⁺).

### (d) 1,1-dimethylethyl(1-{2-[1-methyl-7-(methyloxy)-2-oxo-1,2-dihydro-8-quinolinyl]ethyl}-4-piperidinyl)carbamate

A mixture of [1-methyl-7-(methyloxy)-2-oxo-1,2-dihydro-8-quinolinyl]acetaldehyde (75 mg, 0.3 mmol) and 1,1-dimethylethyl 4-piperidinylcarbamate (71 mg, 0.37 mmol) in dichloromethane (3 mL) and methanol (0.6 mL) was stirred for 24 h before addition of sodium triacetoxyborohydride (200 mg, 0.96 mmol). The reaction was stirred for 1 h before addition of sat. aqueous sodium bicarbonate (2 ml). The reaction was then extracted with 10% methanol in dichloromethane (3×50 ml). The combined organic phases were dried and evaporated, and the crude residue was purified by chromatography on silica gel using a 0-10% methanol/dichloromethane gradient to provide the product as a solid (65 mg, 48%).

MS (+ve ion electrospray) m/z 416 (MH⁺).

### (e) 8-[2-(4-amino-1-piperidinyl)ethyl]-1-methyl-7-(methyloxy)-2(1H)-quinolinone hydrochloride

To a solution of 1,1-dimethylethyl (1-{2-[1-methyl-7-(methyloxy)-2-oxo-1,2-dihydro-8-quinolinyl]ethyl}-4-piperidinyl)carbamate (65 mg, 0.15 mmol) in dichloromethane (3 mL) was added 4M hydrogen chloride in 1,4-dioxane (0.2 mL) and the reaction was stirred at room temperature for 5h before evaporation. The resulting hydrochloride salt (48 mg, 98%) was used without further purification.

MS (+ve ion electrospray) m/z 316 (MH⁺).

### (f) Title Compound

To a solution of 8-[2-(4-amino-1-piperidinyl)ethyl]-1-methyl-7-(methyloxy)-2(1H)-quinolinone hydrochloride salt (48 mg, 0.15 mmol) in dichloromethane (2 mL) and methanol (0.2 mL) was added 3-oxo-3,4-dihydro-2H-pyrido[3,2-b][1,4]thiazine-6-carboxaldehyde (for a synthesis see WO2003087098, Example 301(d)) (30 mg, 0.15 mmol), sodium bicarbonate (0.13 g, 1.5 mmol) and sodium sulphate. The resulting mixture was stirred at room temperature for 24 h before addition of sodium triacetoxyborohydride (99 mg, 0.45 mmol). The reaction was stirred for 1 h and the mixture was then evaporated. The residue was then extracted with 20% methanol in dichloromethane (3×20 ml). The combined organic phases were dried and evaporated, and the crude residue was purified by chromatography on silica gel using a 0-20% methanol/dichloromethane gradient to provide the free base of the title compound as a light orange solid (33 mg, 43%).

MS (+ve ion electrospray) m/z 494 (MH⁺).

¹H NMR (400MHz, CDCl₃) δ 1.58 (2H, m), 1.88-2.09 (2H, m),2.28 (2H, m), 2.60-2.72 (3H, m), 3.12 (2H,m), 3.25 (2H,m), 3.80(2H, s), 3.90(3H, s), 3.95(3H, s), 4.41(2H, t), 6.50 (1H, d), 6.80(1H, m), 7.0(1H, dd), 7.40 (1H, dd), 7.68 (2H, dd).

### Biological Activity

### Antimicrobial Activity Assay:

Whole-cell antimicrobial activity was determined by broth microdilution using the Clinical and Laboratory Standards Institute (CLSI) recommended procedure, Document M7-A7, “Methods for Dilution Susceptibility Tests for Bacteria that Grow Aerobically”. The compounds were tested in serial two-fold dilutions ranging from 0.016 to 16 mcg/mL.

Compounds were evaluated against a panel of Gram-positive organisms, including Staphylococcus aureus, Streptococcus pneumoniae, Streptococcus pyogenes, Enterococcus faecalis and Enterococcus faecium.

In addition, compounds were evaluated against a panel of Gram-negative strains including Haemophilus influenzae, Moraxella catarrhalis, Escherichia coli, Pseudomonas aeruginosa, Proteus mirabilis, Legionella pneumophila, Chlamydia pneumoniae, Enterobacter cloacae, Enterobacter aerogenes, Klebsiella pneumoniae and Stenotrophomonas maltophilia.

The L. pneumophila isolates were tested using a modified CLSI procedure for broth microdilution. For this assay, compounds were tested in serial doubling dilutions over a concentration range of 0.03 to 32 mcg/mL. An inoculum of each test isolate was prepared in buffered yeast broth and adjusted to a density equivalent to a 0.5 McFarland standard. After inoculation, the microtitre plates were incubated at 37° C. for 72 hours.

For the C. pneumoniae isolates, stocks were thawed and diluted in CCM (Chlamydia Culture Media) to yield an inoculum containing ˜1×10⁴ inclusion forming units/ml (IFUs/ml). A 100 μL aliquot of the inoculum was added to all wells of a microtitre plate containing HEp-2 (Human Epithelial (pharyngeal) cell line) cells grown to confluence. Microtitre plates were centrifuged for 1 hour at 1700 g., then incubated for 1 hour at 35° C. in 5% CO₂. One hundred microliters of diluted test compounds, prepared as a 2-fold dilution series in CCM/cycloheximide was then added to the microtiter plates. After 72 hours incubation at 35° C. in 5% CO₂, the microtitre plates were stained with a murine monoclonal fluorescein-conjugated antibody (Kallestad Cat. #532 Roche Biomedical Products) in accordance with the manufacturer recommendations. Upon staining, the IFUs produced an apple-green color, visible against the red counter stained HEp-2 cells when viewed at 100× magnification. The MIC was defined as the lowest concentration of compound at which no IFUs were seen.

The minimum inhibitory concentration (MIC) was determined as the lowest concentration of compound that inhibited visible growth. A mirror reader was used to assist in determining the MIC endpoint.

Each of the listed Examples, as identified in the present application, were tested in at least one exemplified salt form. Unless otherwise noted, the listed Examples had a MIC ≦2 μg/ml against a strain of at least one of the organisms listed above.

1. A compound of formula (I) or a pharmaceutically acceptable salt, solvate or N-oxide thereof: 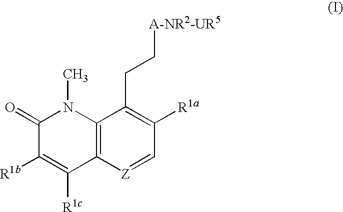
wherein:
Z is C or N;
R^{1a}, R^{1b} and R^{1c} are independently selected from hydrogen; halogen; cyano; (C₁₋₆)alkyl; (C₁₋₆)alkylthio; trifluoromethyl; trifluoromethoxy; carboxy ; hydroxy optionally substituted with (C₁₋₆)alkyl or (C₁₋₆)alkoxy-substituted(C₁₋₆)alkyl; (C₁₋₆)alkoxy-substituted(C₁₋₆)alkyl; hydroxy (C₁₋₆)alkyl; an amino group optionally N-substituted by one or two (C₁₋₆)alkyl, formyl, (C₁₋₆)alkylcarbonyl or (C₁₋₆)alkylsulphonyl groups; or aminocarbonyl wherein the amino group is optionally substituted by (C₁₋₄)alkyl;
provided that when Z is N, R^{1a} is not fluoro;
R² is hydrogen, or (C₁₋₄)alkyl, or together with R⁶ forms Y as defined below;
A is a group (i): 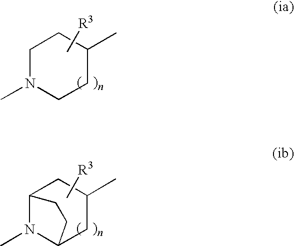
in which: R³ is as defined for R^{1a} or R^{1b} or is oxo and n is 1 or 2:
or A is a group (ii) 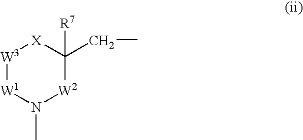
W¹, W² and W³ are CR⁴R⁸
or W² and W³ are CR⁴R⁸ and W¹ represents a bond between W³ and N.
X is O, CR⁴R⁸, or NR⁶;
one R⁴ is as defined for R^{1a}, R^{1b} and R^{1c} and the remainder and R⁸ are hydrogen or one R⁴ and R⁸ are together oxo and the remainder are hydrogen;
R⁶ is hydrogen or (C₁₋₆)alkyl; or together with R² forms Y;
R⁷ is hydrogen; halogen; hydroxy optionally substituted with (C₁₋₆)alkyl; or (C₁₋₆)alkyl;
Y is CR⁴R⁸CH₂; CH₂CR⁴R⁸; (C═O); CR⁴R⁸; CR⁴R⁸(C═O); or (C═O)CR⁴R⁸;
or when X is CR⁴R⁸, R⁸ and R⁷ together represent a bond;
U is selected from CO, and CH₂ and
R⁵ is an optionally substituted bicyclic carbocyclic or heterocyclic ring system (B): 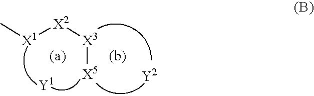
containing up to four heteroatoms in each ring in which
at least one of rings (a)and (b) is aromatic;
X¹ is C or N when part of an aromatic ring, or CR¹⁴ when part of a non-aromatic ring;
X² is N, NR¹³, O, S(O)ₓ, CO or CR¹⁴ when part of an aromatic or non-aromatic ring or may in addition be CR¹⁴R¹⁵ when part of a non aromatic ring;
X³ and X⁵ are independently N or C;
Y¹ is a 0 to 4 atom linker group each atom of which is independently selected from N, NR¹³, O, S(O)ₓ, CO and CR¹⁴ when part of an aromatic or non-aromatic ring or may additionally be CR¹⁴R¹⁵ when part of a non aromatic ring;
Y² is a 2 to 6 atom linker group, each atom of Y² being independently selected from N, NR¹³, O, S(O)ₓ, CO, CR¹⁴ when part of an aromatic or non-aromatic ring or may additionally be CR¹⁴R¹⁵ when part of a non aromatic ring;
each of R¹⁴ and R¹⁵ is independently selected from: H; (C₁₋₄)alkylthio; halo; carboxy(C₁₋₄)alkyl; (C₁₋₄)alkyl; (C_{1 4})alkoxycarbonyl; (C₁₋₄)alkylcarbonyl; (C₁₋₄)alkoxy (C₁₋₄)alkyl; hydroxy; hydroxy(C₁₋₄)alkyl; (C₁₋₄)alkoxy; nitro; cyano; carboxy; amino or aminocarbonyl optionally mono- or di-substituted by (C₁₋₄)alkyl; or
R¹⁴ and R¹⁵ may together represent oxo;
each R¹³ is independently H; trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; (C₁₋₄)alkoxycarbonyl; (C₁₋₄)alkylcarbonyl, (C₁₋₆)alkylsulphonyl; aminocarbonyl wherein the amino group is optionally mono or disubstituted by (C₁₋₄)alkyl;
each x is independently 0, 1 or 2.

2. A compound according to claim 1 wherein R^{1a} is methoxy, cyano, chloro or fluoro and R^{1b} and R^{1c} are hydrogen.

3. A compound according to claim 1 wherein R² is hydrogen.

4. A compound according to claim 1 wherein A is (ia), n is 1 and R³ is H or hydroxy in the 3-position, or A is (ii), X is CR⁴R⁸ and R⁸ is OH and W¹ is a bond, R⁷ is H and W² and W³ are both CH₂.

5. A compound according to claim 1 wherein U is CH₂.

6. A compound according to claim 1 wherein R⁵ is an aromatic heterocyclic ring (B) having 8-11 ring atoms including 2-4 heteroatoms of which at least one is N or NR¹³ in which Y² contains 2-3 heteroatoms, one of which is S and 1-2 are N, with one N bonded to X³, or the heterocyclic ring (B) has ring (a) aromatic selected from optionally substituted benzo, pyrido and pyridazino and ring (b) non aromatic and Y² has 3-4 atoms including at least one heteroatom, with O, S, CH₂ or NR¹³ bonded to X⁵, where R¹³ is other than hydrogen, and either NHCO bonded via N to X³, or O, S, CH₂, or NH bonded to X³.

7. A compound according to claim 1 wherein R⁵ is selected from:
6-substituted 2H-pyrido[3,2-b][1,4]oxazin-3(4H)-one;
2,3-dihydro-[1,4]dioxino[2,3-c]pyridin-7-yl;
[1,3]oxathiolo[5,4-c]pyridin-6-yl;
3,4-dihydro-2H-pyrano[2,3-c]pyridine-6-yl;
6-substituted 2H-pyrido[3,2-b][1,4]thiazin-3(4H)-one;
6-substituted 7-chloro-2H-pyrido[3,2-b][1,4]oxazin-3(4H)-one; and
6,7-dihydro[1,4]dioxino[2,3-c]pyridazin-3-yl,

8. A compound selected from:
7-chloro-6-[({1-[2-(3-chloro-5-methyl-6-oxo-5,6-dihydro-1,5-naphthyridin-4-yl)ethyl]-4-piperidinyl}amino)methyl]-2H-pyrido[3,2-b][1,4]oxazin-3(4H)-one;
7-chloro-1-methyl-8-(2-{4-[([1,3]oxathiolo[5,4-c]pyridin-6-ylmethyl)amino]-1-piperidinyl}ethyl)-1,5-naphthyridin-2(1H)-one;
7-chloro-8-[2-((3S,4S)-3-hydroxy-4-{[([1,3]oxathiolo[5,4-c]pyridin-6-ylmethyl)amino]methyl}-1-pyrrolidinyl)ethyl]-1-methyl-1,5-naphthyridin-2(1H)-one;
6-[({1-[2-(3-chloro-5-methyl-6-oxo-5,6-dihydro-1,5-naphthyridin-4-yl)ethyl]-4-piperidinyl}amino)methyl]-2H-pyrido[3,2-b][1,4]thiazin-3(4H)-one;
6-[({(3R,4S)-1-[2-(3-chloro-5-methyl-6-oxo-5,6-dihydro-1,5-naphthyridin-4-yl)ethyl]-3-hydroxy-4-piperidinyl}amino)methyl]-2H-pyrido[3,2-b][1,4]oxazin-3(4H)-one;
7-chloro-1-methyl-8-[2-((2S)-2-{[([1,3]oxathiolo[5,4-c]pyridin-6-ylmethyl)amino]methyl}-4-morpholinyl)ethyl]-1,5-naphthyridin-2(1H)-one;
6-{[({(3S,4S)-1-[2-(3-chloro-5-methyl-6-oxo-5,6-dihydro-1,5-naphthyridin-4-yl)ethyl]-4-hydroxy-3-pyrrolidinyl}methyl)amino]methyl}-2H-pyrido[3,2-b][1,4]thiazin-3(4H)-one;
7-chloro-1-methyl-8-[2-((3R)-3-{[([1,3]oxathiolo[5,4-c]pyridin-6-ylmethyl)amino]methyl}-1-pyrrolidinyl)ethyl]-1,5-naphthyridin-2(1H)-one;
7-chloro-1-methyl-8-[2-((3S)-3-{[([1,3]oxathiolo[5,4-c]pyridin-6-ylmethyl)amino]methyl}-1-pyrrolidinyl)ethyl]-1,5-naphthyridin-2(1H)-one;
6-[({1-[2-(3-Chloro-5-methyl-6-oxo-5,6-dihydro-1,5-naphthyridin-4-yl)ethyl]-4-piperidinyl}amino)methyl]-2H-pyrido[3,2-b][1,4]oxazin-3(4H)-one;
7-chloro-8-(2-{4-[(2,3-dihydro[1,4]dioxino[2,3-c]pyridin-7-ylmethyl)amino]-1-piperidinyl}ethyl)-1-methyl-1,5-naphthyridin-2(1H)-one;
7-chloro-8-(2-{4-[(3,4-dihydro-2H-pyrano[2,3-c]pyridin-6-ylmethyl)amino]-1-piperidinyl}ethyl)-1-methyl-1,5-naphthyridin-2(1H)-one;
5-[({(3R,4S)-1-[2-(3-chloro-5-methyl-6-oxo-5,6-dihydro-1,5-naphthyridin-4-yl)ethyl]-3-hydroxy-4-piperidinyl}amino)methyl]-2,3-dihydro-1-benzofuran-7-carbonitrile;
6-{[({(3R)-1-[2-(3-chloro-5-methyl-6-oxo-5,6-dihydro-1,5-naphthyridin-4-yl)ethyl]-3-pyrrolidinyl}methyl)amino]methyl}-2H-pyrido[3,2-b][1,4]oxazin-3(4H)-one;
6-{[({(3R)-1-[2-(3-chloro-5-methyl-6-oxo-5,6-dihydro-1,5-naphthyridin-4-yl)ethyl]-3-pyrrolidinyl}methyl)amino]methyl}-2H-pyrido[3,2-b][1,4]thiazin-3(4H)-one;
6-{[({(3R,4R)-1-[2-(3-chloro-5-methyl-6-oxo-5,6-dihydro-1,5-naphthyridin-4-yl)ethyl]-4-hydroxy-3-pyrrolidinyl}methyl)amino]methyl}-2H-pyrido[3,2-b][1,4]thiazin-3(4H)-one;
7-{[({(3R)-1-[2-(3-chloro-5-methyl-6-oxo-5,6-dihydro-1,5-naphthyridin-4-yl)ethyl]-3-pyrrolidinyl}methyl)amino]methyl}-2,3-dihydro-1,4-benzodioxin-5-carbonitrile;
7-chloro-8-[2-((3R)-3-{[(2,3-dihydro[1,4]dioxino[2,3-c]pyridin-7-ylmethyl)amino]methyl}-1-pyrrolidinyl)ethyl]-1-methyl-1,5-naphthyridin-2(1H)-one;
6-[({1-[2-(5-methyl-6-oxo-5,6-dihydro-1,5-naphthyridin-4-yl)ethyl]-4-piperidinyl}amino)methyl]-2H-pyrido[3,2-b][1,4]thiazin-3(4H)-one;
8-(2-{4-[(2,3-dihydro[1,4]dioxino[2,3-c]pyridin-7-ylmethyl)amino]-1-piperidinyl}ethyl)-1-methyl-1,5-naphthyridin-2(1H)-one;
1-methyl-8-(2-{4-[([1,3]oxathiolo[5,4-c]pyridin-6-ylmethyl)amino]-1-piperidinyl}ethyl)-1,5-naphthyridin-2(1H)-one;
8-(2-{4-[(2,3-dihydro[1,4]dioxino[2,3-c]pyridin-7-ylmethyl)amino]-1-piperidinyl}ethyl)-1-methyl-2(1H)-quinolinone;
5-methyl-4-(2-{4-[([1,3]oxathiolo[5,4-c]pyridin-6-ylmethyl)amino]-1-piperidinyl}ethyl)-6-oxo-5,6-dihydro-1,5-naphthyridine-3-carbonitrile;
4-(2-{4-[(2,3-dihydro[1,4]dioxino[2,3-c]pyridin-7-ylmethyl)amino]-1-piperidinyl}ethyl)-5-methyl-6-oxo-5,6-dihydro-1,5-naphthyridine-3-carbonitrile;
5-methyl-6-oxo-4-[2-(4-{[(3-oxo-3,4-dihydro-2H-pyrido[3,2-b][1,4]thiazin-6-yl)methyl]amino}-1-piperidinyl)ethyl]-5,6-dihydro-1,5-naphthyridine-3-carbonitrile;
4-[2-((3R,4S)-3-hydroxy-4-{[(3-oxo-3,4-dihydro-2H-pyrido[3,2-b][1,4]thiazin-6-yl)methyl]amino}-1-piperidinyl)ethyl]-5-methyl-6-oxo-5,6-dihydro-1,5-naphthyridine-3-carbonitrile;
4-(2-{(3R,4S)-4-[(2,3-dihydro[1,4]dioxino[2,3-c]pyridin-7-ylmethyl)amino]-3-hydroxy-1-piperidinyl}ethyl)-5-methyl-6-oxo-5,6-dihydro-1,5-naphthyridine-3-carbonitrile;
4-(2-{(3R,4S)-3-hydroxy-4-[([1,3]oxathiolo[5,4-c]pyridin-6-ylmethyl)amino]-1-piperidinyl}ethyl)-5-methyl-6-oxo-5,6-dihydro-1,5-naphthyridine-3-carbonitrile;
4-{2-[(3S,4S)-3-hydroxy-4-({[(3-oxo-3,4-dihydro-2H-pyrido[3,2-b][1,4]thiazin-6-yl)methyl]amino}methyl)-1-pyrrolidinyl]ethyl}-5-methyl-6-oxo-5,6-dihydro-1,5-naphthyridine-3-carbonitrile;
4-[2-((3S,4S)-3-hydroxy-4-{[([1,3]oxathiolo[5,4-c]pyridin-6-ylmethyl)amino]methyl}-1-pyrrolidinyl)ethyl]-5-methyl-6-oxo-5,6-dihydro-1,5-naphthyridine-3-carbonitrile;
8-(2-{4-[(6,7-dihydro[1,4]dioxino[2,3-c]pyridazin-3-ylmethyl)amino]-1-piperidinyl}ethyl)-7-fluoro-1-methyl-2(1H)-quinolinone;
6-[({1-[2-(7-fluoro-1-methyl-2-oxo-1,2-dihydro-8-quinolinyl)ethyl]-4-piperidinyl}amino)methyl]-2H-pyrido[3,2-b][1,4]oxazin-3(4H)-one;
8-(2-{4-[(2,3-dihydro[1,4]dioxino[2,3-c]pyridin-7-ylmethyl)amino]-1-piperidinyl}ethyl)-7-fluoro-1-methyl-2(1H)-quinolinone;
7-fluoro-1-methyl-8-(2-{4-[([1,3]oxathiolo[5,4-c]pyridin-6-ylmethyl)amino]-1-piperidinyl}ethyl)-2(1H)-quinolinone;
3-{[{1-[2-(7-fluoro-1-methyl-2-oxo-1,2-dihydro-8-quinolinyl)ethyl]-4-piperidinyl}(methyl)amino]methyl}-5H-pyridazino[3,4-b][1,4]thiazin-6(7H)-one; and
6-{[(1-{2-[1-methyl-7-(methyloxy)-2-oxo-1,2-dihydro-8-quinolinyl]ethyl}-4-piperidinyl)amino]methyl}-2H-pyrido[3,2-b][1,4]thiazin-3(4H)-one;
or a pharmaceutically acceptable salt thereof.

9. A method of treatment of bacterial infections in mammals, which method comprises the administration to a mammal in need of such treatment an effective amount of a compound according to claim 1.

10. (canceled)

11. A pharmaceutical composition comprising a compound according to claim 1 and a pharmaceutically acceptable carrier.

12. A compound according to claim 4 wherein A is 3-hydroxypyrrolidin-4-ylmethyl and the configuration is (3S,4S).

13. A compound according to claim 1 wherein:
R^{1a} is methoxy, cyano, chloro or fluoro and R^{1b} and R^{1c} are hydrogen;
R² is hydrogen;
A is (ia), n is 1 and R³ is H or hydroxy in the 3-position, or A is (ii), X is CR⁴R⁸ and R⁸ is OH and W¹ is a bond. R⁷ is H and W² and W³ are both CH₂;
U is CH₂; and
R⁵ is an aromatic heterocyclic ring (B) having 8-11 ring atoms including 2-4 heteroatoms of which at least one is N or NR¹³ in which Y² contains 2-3 heteroatoms, one of which is S and 1-2 are N, with one N bonded to X³, or the heterocyclic ring (B) has ring (a) aromatic selected from optionally substituted benzo, pyrido and pyridazino and ring (b) non aromatic and Y² has 3-4 atoms including at least one heteroatom, with O, S, CH₂ or NR¹³ bonded to X⁵, where R¹³ is other than hydrogen, and either NHCO bonded via N to X³, or O, S, CH₂, or NH bonded to X³.

14. A compound according to claim 13 wherein R⁵ is selected from:
6-substituted 2H-pyrido[3,2-b][1,4]oxazin-3(4H)-one;
2,3-dihydro-[1,4]dioxino[2,3-c]pyridin-7-yl;
[1,3]oxathiolo[5,4-c]pyridin-6-yl;
3,4-dihydro-2H-pyrano[2,3-c]pyridine-6-yl;
6-substituted 2H-pyrido[3,2-b][1,4]thiazin-3(4H)-one;
6-substituted 7-chloro-2H-pyrido[3,2-b][1,4]oxazin-3(4H)-one; and
6,7-dihydro[1,4]dioxino[2,3-c]pyridazin-3-yl.

15. A method according to claim 9 comprising the administration to a mammal in need of such treatment an effective amount of a compound according to claim 8.

16. A method according to claim 9 comprising the administration to a mammal in need of such treatment an effective amount of a compound according to claim 14.

17. A method according to claim 9 wherein the mammal is a human.

18. A method according to claim 15 wherein the mammal is a human.

19. A method according to claim 16 wherein the mammal is a human.

20. A pharmaceutical composition comprising a compound according to claim 8 and a pharmaceutically acceptable carrier.

21. A pharmaceutical composition comprising a compound according to claim 14 and a pharmaceutically acceptable carrier.
